(19) United States
(12) Patent Application Publication
(10) Pub. No.: US 2025/0017936 A1
(43) Pub. Date: Jan. 16, 2025

(54) CELL-POTENT BISUBSTRATE INHIBITORS FOR NICOTINAMIDE N-METHYLTRANSFERASE (NNMT) AND USES THEREOF

(71) Applicant: PURDUE RESEARCH FOUNDATION, West Lafayette, IN (US)

(72) Inventors: Rong HUANG, West Lafayette, IN (US); Iredia IYAMU, West Lafayette, IN (US)

(21) Appl. No.: 18/712,613

(22) PCT Filed: Nov. 21, 2022
(86) PCT No.: PCT/US2022/050634

§ 371 Date: May 22, 2024

Related U.S. Application Data
(60) Provisional application No. 63/281,788, filed on Nov. 22, 2021.

(51) Int. Cl. A61K 31/519; A61P 35/00; C07D 487/04

(52) U.S. Cl. CPC A61K 31/519; A61P 35/00; C07D 487/04

(57) ABSTRACT

A compound with the following formula (I) or a pharmaceutically acceptable salt thereof; a pharmaceutical composition comprising a compound of formula (I); and a method of inhibiting nicotinamide N-methyItransferase in a patient in need thereof, such as a patient with cancer.

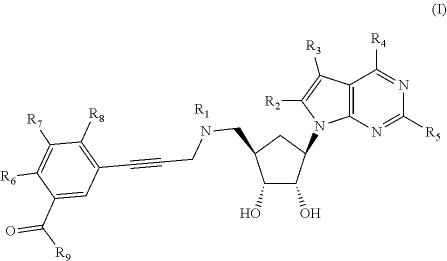

### CROSS REFERENCE TO RELATED APPLICATION

This application claims priority to U.S. provisional patent application no. 63/281,788, which was filed Nov. 22, 2021, and which is hereby incorporated by reference in its entirety.

### TECHNICAL FIELD

The present disclosure generally relates to new compounds as a therapeutic intervention, particularly to a class of small molecular cell-potent bisubstrate inhibitors for nicotinamide N-methyltransferase (NNMT) and the uses thereof.

### BACKGROUND

Methylation of proteins or small molecules plays important role in diverse processes including transcription, metabolism, and DNA damage repair (Gokul et al., Subcell Biochem 61:597-625 (2013); and Jin et al., Adv Exp med Biol 754:3-29 (2013)). Thus, methyltransferases have emerged as pivotal targets for numerous diseases (Kraus et al. (2014), supra; Parsons et al. (2003), supra; Kocinaj et al. (2021), supra; Chlopicki et al., Br J Pharmacol 152:230-239 (2007); and Eckert et al. (2019), supra).

Nicotinamide N-methyltransferase (NNMT) catalyzes the methyl group transfer from S-adenosyl-L-methionine (SAM) to nicotinamide (NAM), while generating S-adenosyl-L-homocysteine (SAH) and N1-methyl nicotinamide (MNA) (Alston et al., Arch Biochem Biophys 260: 601-608 (1988); and Hong et al., Biochem 57: 5775-5779 (2018)). Elevated levels of NNMT have been implicated in various cancers as well as metabolic, cardiovascular, liver, and neurodegenerative diseases (Kraus et al., Nature 508:258-262(2014); Parsons et al., Neurosci Lett 342: 13-16 (2003); Kocinaj et al., Mol Neurobiol 58: 1769-1781 (2021); Eckert et al., Nature 569: 723-728 (2019); Panichamy et al., Clin Cancer Res 23: 2325-2334 (2017); Jung et al., J Clin Invest 2: 1-23 (2017); Tang et al., Carcinogenesis 32: 138-145 (2011); Ulanovskaya et al., Nat Chem Biol 9: 300-306 (2013); and Iyamu et al., RSC Med Chem 12: 1254-1261 (2021); and Griffiths et al., Am J Physiol Cell Physiol 321: C585-595 (2021)), establishing NNMT as a promising therapeutic target for aforementioned diseases.

Despite the feasibility of targeting a single substrate binding site, bisubstrate analogs that covalently link a substrate analog with a cofactor analog to mimic the transition state remain an appreciable strategy to produce new lead compounds with decent potency and selectivity for further optimization (Huang et al., Nat Struct Mol Biol 77: 646-649(2010)). Many efforts have been made to build bisubstrate inhibitors for methyltransferases to achieve simultaneous targeting of both substrate and cofactor binding sites. Several successful examples of low nanomolar bisubstrate inhibitors have been reported for protein N-terminal methyltransferase 1 (NTMT1), nicotinamide N-methyltransferase (NNMT), protein arginine methyltransferases (PRMTs), phenylethanolamine N-methyltransferase (PNMT), and catechol-O-methyltransferase (COMT) (Paulini et al., Chem Med Chem 1: 340-357 (2006); Mahmoodi et al., J Am Chem Soc 142: 14222-14233 (2020); Chen et al., J Med Chem 62: 3773-3779 (2019); Chen et al., J Med Chem 62: 10783-10797 (2019); and Iyamu et al. (2021), supra). Besides exquisite selectivity against closely-related homologs, the advantage of the aforementioned potent bisubstrate inhibitors is their boosted residence time accompanied by tight-binding affinities.

In view of the above, it is an object of the present disclosure to provide small molecule, bisubstrate inhibitors for NNMT, which have improved selectivity and potency. This and other objects and advantages, as well as inventive features, will be apparent from the detailed description provided herein.

### SUMMARY

Provided is a compound having the formula (I):

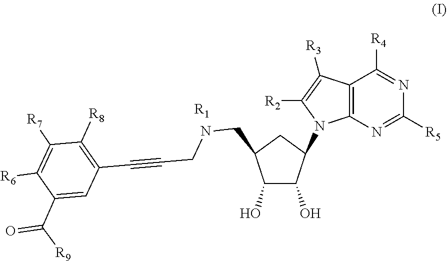

or a pharmaceutically acceptable salt thereof, wherein,
R₁ is an alkyl, a haloalkyl, an alkenyl, an alkynyl, an aminoalkanoic acid, an acyl, an arylalkylacyl, an arylacyl, a cycloalkyl, a heterocyclyl, an aryl, a heteroaryl, an arylalkyl, or a heteroarylalkyl;

R₂, R₃, R₄, and R₅ are, independently, hydrogen, a halo, a haloalkyl, an alkyl, an amino, an alkylamino, or an alkylaminoalkyl;
R₆, R₇, and R₈ are, independently, hydrogen, methyl, hydroxyl, methoxyl, trifluoromethyl, or a halo; and
R₉ is an amine or methyl,
wherein R₁-R₆ are, independently, optionally substituted with any other substituent. The compound can be:

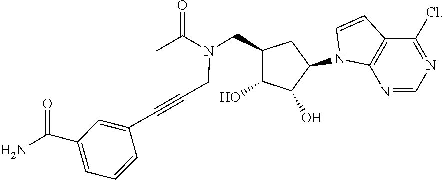 [SMILES: CC(=O)N(CC#Cc1cccc(C(N)=O)c1)C[C@H]1C[C@@H](n2ccc3c(Cl)ncnc32)[C@H](O)[C@@H]1O]

The compound can be:

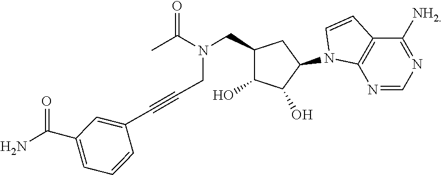 [SMILES: CC(=O)N(CC#Cc1cccc(C(N)=O)c1)C[C@H]1C[C@@H](n2ccc3c(N)ncnc32)[C@H](O)[C@@H]1O]

The compound can be:

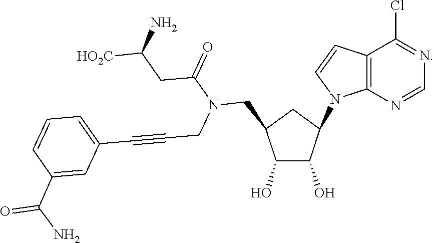

The compound can be:

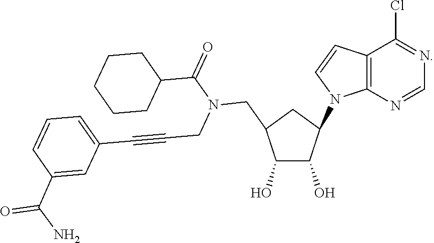

The compound can be:

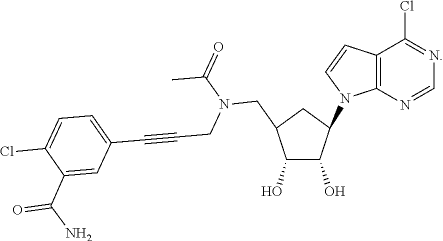

The compound can be:

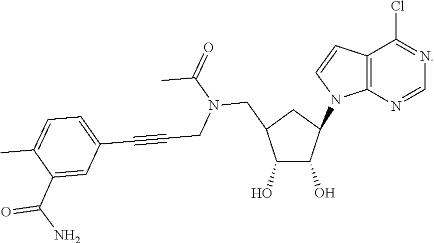

The compound can be:

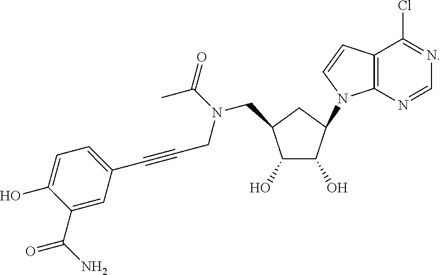 [SMILES: CC(=O)N(CC#Cc1ccc(O)c(C(N)=O)c1)C[C@H]1C[C@@H](n2ccc3c(Cl)ncnc32)[C@H](O)[C@@H]1O]

The compound can be:

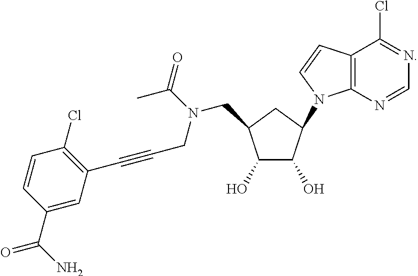 [SMILES: CC(=O)N(CC#Cc1cc(C(N)=O)ccc1Cl)C[C@H]1C[C@@H](n2ccc3c(Cl)ncnc32)[C@H](O)[C@@H]1O]

The compound can be:

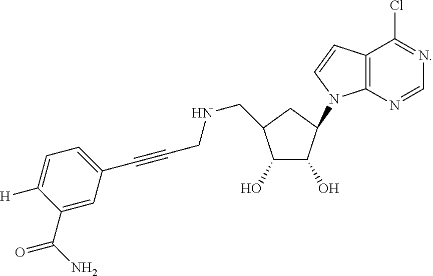 [SMILES: NC(=O)c1cccc(C#CCNCC2C[C@@H](n3ccc4c(Cl)ncnc43)[C@H](O)[C@@H]2O)c1]

The compound can be, and desirably is, a cell-potent inhibitor for nicotinamide N-methyltransferase (NNMT), a bisubstrate for NNMT and its cofactor, and selective for NNMT over other closely related methyltransferases.

Further provided is a pharmaceutical composition comprising one or more of the above-described compounds, together with one or more pharmaceutically acceptable diluents, excipients, or carriers. The one or more compounds can be, and desirably are, cell-potent inhibitors for NNMT, bisubstrates for NNMT and its cofactor, and selective for NNMT over other closely related methyltransferases.

Also provided is a method for inhibiting NNMT in a subject in need thereof. The method comprises administering to the subject an NNMT-inhibiting effective amount of an above-described compound or a pharmaceutical composition comprising same, together with one or more pharmaceutically acceptable diluents, excipients, or carriers. The compound can be, and desirably is, a cell-potent inhibitor for NNMT, a bisubstrate for NNMT and its cofactor, and selective for NNMT over other closely related methyltransferases. The subject can have cancer. The subject can have diabetes, a liver disease, scleroderma, or Parkinson's disease.

Also provided is a compound as shown in any one of FIGS. 17A-17H or a pharmaceutical composition comprising same, together with one or more pharmaceutically acceptable diluents, excipients, or carriers. The compound can be, and desirably is, a cell-potent inhibitor for NNMT, a bisubstrate for NNMT and its cofactor, and selective for NNMT over other closely related methyltransferases.

In view of the above, also provided is a method for inhibiting nicotinamide N-methyltransferase (NNMT) in a subject in need thereof. The method comprises administering to the subject an NNMT-inhibiting effective amount of the aforementioned compound (FIGS. 17A-17H) or a pharmaceutical composition comprising same. The subject can have cancer. The subject can have diabetes, a liver disease, scleroderma, or Parkinson's disease.

### BRIEF DESCRIPTION OF THE DRAWINGS

FIG. 1A shows the co-crystal structure of LL320 bound to NNMT (NNMT-LL320; PDB: 6PVS). Heteroatoms that make no direct interactions with NNMT are marked with asterisks.

FIG. 1B shows II399 which was rationally designed based on the co-crystal structure of FIG. 1A.

FIG. 2A shows the fluorescence polarization (mP) results of a competition assay with the indicated compounds at varying concentrations (log conc. (μM)). All experiments were performed in duplicates (n=2).

FIG. 2B shows activity (%) of the indicaed compounds at varying concentrations (μM) in a S-adenosyl-L-homocysteine hydrolase (SAHH)-coupled fluorescence assay. All experiments were performed in duplicates (n=2).

FIG. 3A shows a ligand interaction diagram of II399 with NNMT (PDB ID: 7RKK & 7RKL).

FIG. 3B shows a 2Fo-Fc omit map contoured to 1.0 σ for II399 as a transparent green isosurface.

FIG. 3C shows a structural alignment of II399 and LL320 within the binding pocket of NNMT.

FIG. 4A shows binding affinities of II399 for NNMT through isothermal titration calorimetry (ITC) experiment (n=2).

FIG. 4B shows binding affinities of LL320 for NNMT through ITC experiment (n=2).

FIG. 5A shows IC₅₀ curves of II399 at varying concentrations of substrate nicotinamide (NAM) with a fixed concentration of S-adenosyl-L-homocysteine (SAM) at 10 μM.

FIG. 5B shows a linear regression plot of IC₅₀ values with corresponding concentrations of NAM.

FIG. 5C shows IC₅₀ curves of II399 at varying concentrations of SAM with a fixed concentration of NAM at 10 μM.

FIG. 5D shows a linear regression plot of IC₅₀ values with corresponding concentrations of SAM.

FIG. 5E shows an IC₅₀ curve of II399 at 4 Kₘ concentrations of both SAM and substrate in the S-adenosyl-L-homocysteine hydrolase (SAHH)-coupled fluorescence assay.

FIG. 5F shows a concentration-response plot for compound II399 against phenylethanolamine N-methyltransferase (PNMT), PRMT1 (from protein arginine methyltransferase (PRMT)), G9a (from protein lysine methyltransaferase (PKMT)), N-terminal methyltransferase 1 (NTMT1), and SAHH with each compound at four different concentrations (100, 33.3, 11.1, and 3.7 μM). All experiments were performed in duplicates (n=2).

FIG. 6 shows a concentration-response plot of II399 inhibition on cellular N1-methyl nicotinamide (MNA) levels in 769P cells. (n=2).

FIG. 7 shows the structure-guided design of cell-potent inhibitors based on the interaction of LL320 bound to NNMT (NNMT-LL320; PDB: 6PVS). Heteroatoms making no direct interactions with NNMT are marked with asterisks.

FIG. 8A shows IC₅₀ curves of II559 at varying concentrations of substrate (NAM) with a fixed concentration of SAM at 10 μM. All experiments were performed in duplicates (n=2).

FIG. 8B shows a linear regression plot of IC₅₀ values with corresponding concentrations of substrate (NAM). All experiments were performed in duplicates (n=2).

FIG. 8C shows IC₅₀ curves of II559 at varying concentrations of SAM with a fixed concentration of substrate (NAM) at 10 μM. All experiments were performed in duplicates (n=2).

FIG. 8D shows a linear regression plot of IC₅₀ values with corresponding concentrations of SAM. All experiments were performed in duplicates (n=2).

FIG. 8E shows docking of II399 in the active site of NNMT (PDB ID: 7RKL). Kᵢ=5.9 nM.

FIG. 8F shows docking of II559 in the active site of NNMT (PDB ID: 7RKL). Kᵢ=1.2 nM.

FIG. 8G shows docking of II562 in the active site of NNMT (PDB ID: 7RKL). Kᵢ=3.1 nM.

FIG. 9A shows SAHH-coupled fluorescence assay of compound II559 at 4 Kₘ SAM and a substrate concentration ranging from 0 to 10 μM. All experiments were performed in duplicates (n=2).

FIG. 9B shows a concentration-response plot for compound II559. All experiments were performed in duplicates (n=2).

FIG. 10A shows thermal stabilization of NNMT by immunoblotting using an anti-NNMT antibody (proteintech 15123-1-AP) when treated with DMSO or II559 (10 μM) for 4 hours.

FIG. 10B shows quantification of the relative NNMT band intensities of the immunoblot of FIG. 10A in duplicate. Tagg=apparent aggregation temperature.

FIG. 10C shows thermal stabilization of NNMT by immunoblotting using an anti-NNMT antibody (proteintech 15123-1-AP) when treated with DMSO or II562 (10 μM) for 3 hours.

FIG. 10D shows quantification of the relative NNMT band intensities of the immunoblot of FIG. 10C in duplicate. Tagg=apparent aggregation temperature.

FIG. 11 shows selectivity studies for compounds II559 against PNMT, PRMT1, TbPRMT7 (from PRMT), G9a, SETD7 (from PKMT), NTMT1, and SAHH with each compound at four different concentrations (100, 33.3, 11.1, and 3.7 μM). All experiments were performed in duplicates (n=2).

FIG. 12A shows the cellular concentration-response plot for the cell-potent inhibitor II562.

FIG. 12B shows the cellular concentration-response plot for the cell-potent inhibitor II559.

FIG. 13A shows IC₅₀ curves at 9 Kₘ concentrations of both SAM and substrate in the SAHH-coupled fluorescence assay. Kᵢ was calculated using the equation Kᵢ=IC₅₀/(1+[S]/Kₘ).

FIG. 13B shows the results of selectivity studies for II559 against PNMT, PRMT1 TbPRMT7, G9a, SETD7, NTMT1, and SAHH with each compound at four different concentrations (100, 33.3, 11.1, and 3.7 μM). All experiments were performed in duplicates (n=2). Values were presented as mean±standard deviation (SD).

FIG. 13C shows the results of selectivity studies for II562 against PNMT, PRMT1 ThPRMT7, G9a, SETD7, NTMT1, and SAHH with each compound at four different concentrations (100, 33.3, 11.1, and 3.7 μM). All experiments were performed in duplicates (n=2). Values were presented as mean±standard deviation (SD).

FIG. 14A shows effect of NNMT inhibition on cell viability of renal cancer cell line 769P after incubation with inhibitor for 10 days.

FIG. 14B shows effect of NNMT inhibition on cell viability of renal cancer cell line 786O after incubation with inhibitor for 10 days.

FIG. 15 is a schematic diagram of structure-activity relationship studies of the cell-potent NNMT bisubstrate inhibitor and II559.

FIG. 16 shows the design strategy of bisubstrate inhibitors based on LL320.

FIGS. 17A, 17B, and 17C show inhibitors that are based on LL320 with R1 group modifications.

FIGS. 17D, 17E, and 17F show inhibitors that are based on LL320 with R2 group modifications.

FIGS. 17G and 17H show inhibitors that are based on LL320 with R3 group modifications.

For the purposes of promoting an understanding of the principles of the present disclosure, reference will now be made to the embodiments illustrated in the drawings, and specific language will be used to describe the same. No limitation of the scope of claimed invention is thereby intended.

### DETAILED DESCRIPTION

The present disclosure is predicated on the discovery of cell-potent nicotinamide N-methyltransferase (NNMT) bisubstrate inhibitors. II399 demonstrates a Kᵢ of 5.9 nM in a biochemical assay and a cellular IC₅₀ value of 2.0 μM. The inhibition mechanism and cocrystal structure of II399-NNMT confirmed that II399 engages with both the substrate and cofactor binding pockets of NNMT. Computational modeling and isothermal titration calorimetry data revealed a balancing act between enthalpic and entropic components of binding that lead to II399's low nM binding affinity. Notably, II399 is 10,000-fold more selective for NNMT than closely related methyltransferases. Compound II559 has a Kᵢ of 1.2±0.04 nM in a biochemical assay and a cellular IC₅₀ value of 0.14 μM, representing the most cell-potent inhibitor to date.

In view of the above, provided is a compound having the formula (I):

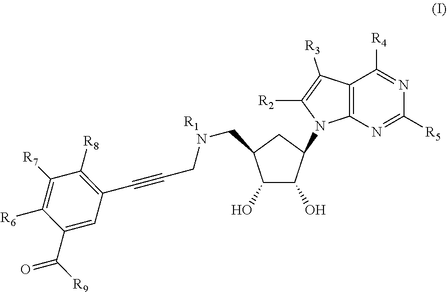

or a pharmaceutically acceptable salt thereof, wherein,
R₁ is an alkyl, a haloalkyl, an alkenyl, an alkynyl, an aminoalkanoic acid, an acyl, an arylalkylacyl, an arylacyl, a cycloalkyl, a heterocyclyl, an aryl, a heteroaryl, an arylalkyl, or a heteroarylalkyl;
R₂, R₃, R₄, and R₅ are, independently, hydrogen, a halo, a haloalkyl, an alkyl, an amino, an alkylamino, or an alkylaminoalkyl;
R₆, R₇, and R₈ are, independently, hydrogen, methyl, hydroxyl, methoxyl, trifluoromethyl, or a halo; and
R₉ is an amine or methyl,
   wherein R₁-R₆ are, independently, optionally substituted with any other substituent.
   The compound can be:

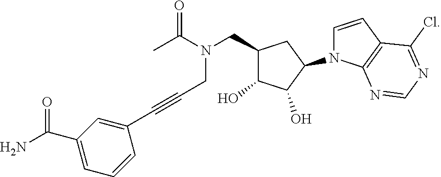 [SMILES: CC(=O)N(CC#Cc1cccc(C(N)=O)c1)C[C@H]1C[C@@H](n2ccc3c(Cl)ncnc32)[C@H](O)[C@@H]1O]

The compound can be:

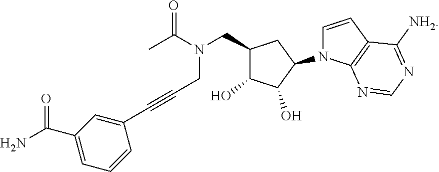 [SMILES: CC(=O)N(CC#Cc1cccc(C(N)=O)c1)C[C@H]1C[C@@H](n2ccc3c(N)ncnc32)[C@H](O)[C@@H]1O]

The compound can be:

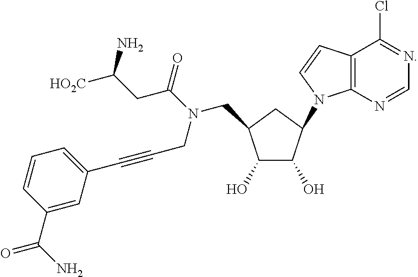 [SMILES: NC(=O)c1cccc(C#CCN(C[C@H]2C[C@@H](n3ccc4c(Cl)ncnc43)[C@H](O)[C@@H]2O)C(=O)C[C@H](N)C(=O)O)c1]

The compound can be:

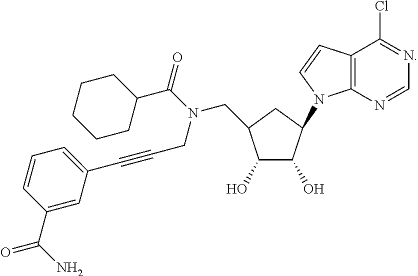 [SMILES: NC(=O)c1cccc(C#CCN(CC2C[C@@H](n3ccc4c(Cl)ncnc43)[C@H](O)[C@@H]2O)C(=O)C2CCCCC2)c1]

The compound can be:

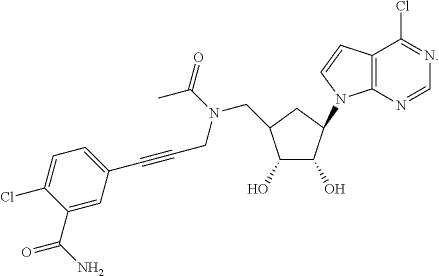 [SMILES: CC(=O)N(CC#Cc1ccc(Cl)c(C(N)=O)c1)CC1C[C@@H](n2ccc3c(Cl)ncnc32)[C@H](O)[C@@H]1O]

The compound can be:

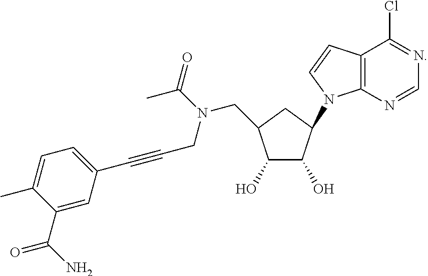 [SMILES: CC(=O)N(CC#Cc1ccc(C)c(C(N)=O)c1)CC1C[C@@H](n2ccc3c(Cl)ncnc32)[C@H](O)[C@@H]1O]

The compound can be:

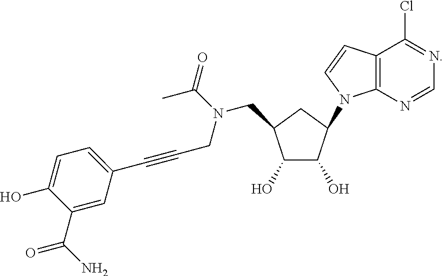 [SMILES: CC(=O)N(CC#Cc1ccc(O)c(C(N)=O)c1)C[C@H]1C[C@@H](n2ccc3c(Cl)ncnc32)[C@H](O)[C@@H]1O]

The compound can be:

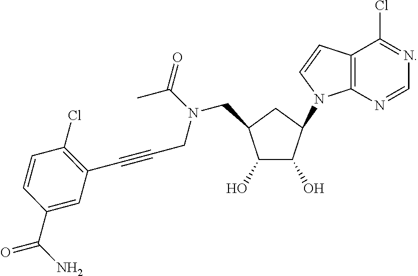 [SMILES: CC(=O)N(CC#Cc1cc(C(N)=O)ccc1Cl)C[C@H]1C[C@@H](n2ccc3c(Cl)ncnc32)[C@H](O)[C@@H]1O]

The compound can be:

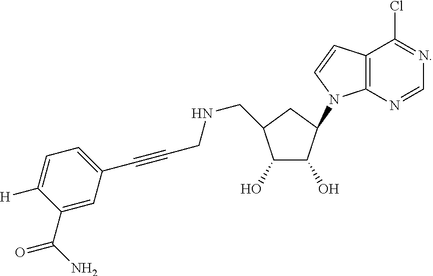 [SMILES: NC(=O)c1cccc(C#CCNCC2C[C@@H](n3ccc4c(Cl)ncnc43)[C@H](O)[C@@H]2O)c1]

The compound can be, and desirably is, a cell-potent inhibitor for NNMT, a bisubstrate for NNMT and its cofactor, and selective for NNMT over other closely related methyltransferases.

The compounds can be synthesized in accordance with methods known in the art. Other methods are exemplified herein.

In each of the foregoing and following embodiments, the formulae include and represent not only all pharmaceutically acceptable salts of the compounds, but also include any and all hydrates and/or solvates of the compound formulae or salts thereof. Certain functional groups, such as the hydroxy, amino, and like groups form complexes and/or coordination compounds with water and/or various solvents, in the various physical forms of the compounds. Accordingly, the formulae include and represent those various hydrates and/or solvates. The formulae also include and represent each possible isomer, such as stereoisomers and geometric isomers, both individually and in any and all possible mixtures. The formulae also include and represent any and all crystalline forms, partially crystalline forms, and non-crystalline and/or amorphous forms of the compounds.

The compounds may contain one or more chiral centers, or may otherwise be capable of existing as multiple stereoisomers. The disclosure is not limited to any particular stereochemical requirement, and the compounds may be optically pure or may be any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configurations at one or more other chiral centers.

Similarly, the compounds may include geometric centers, such as cis and trans, e.g., E and Z, double bonds. The disclosure is not limited to any particular geometric isomer requirement, and the compounds may be pure or may be any of a variety of geometric isomer mixtures. Such mixtures of geometric isomers may include a single configuration at one or more double bonds, while including mixtures of geometry at one or more other double bonds.

The term “positional isomer” refers to structural isomers around a central ring, such as ortho-, meta-, and para-isomers around a benzene ring. Further, it is understood that replacement of one or more hydrogen atoms with deuterium can significantly lower the rate of metabolism of a drug and, therefore, increase its half-life.

The term “organic group” refers to, but is not limited to, any carbon-containing functional group. For example, an oxygen-containing group, such as an alkoxy group, aryloxy group, aralkyloxy group, oxo (carbonyl) group, a carboxyl group (including a carboxylic acid, carboxylate, and a carboxy late ester), a sulfur-containing group (including an alkyl sulfide and an aryl sulfide), or another aheteroatom-containing group.

The term “substituted” refers to an organic group or molecule in which one or more hydrogen atoms is/are replaced with one or more non-hydrogen atoms. The term “functional group” or “substituent” refers to a group that can be or is substituted onto a molecule or onto an organic group. Examples of substituents or functional groups include, but are not limited to, a halogen (e.g., F, Cl, Br, and I); an oxygen atom in groups such as a hydroxyl group, an alkoxy group, an aryloxy group, an aralkyloxy group, an oxo (carbonyl) group, and a carboxyl group (including a carboxylic acid, a carboxylate, and a carboxylate ester); a sulfur atom in groups such as a thiol group, an alkyl sulfide group, an aryl sulfide group, a sulfoxide group, a sulfone group, a sulfonyl group, and a sulfonamide group; a nitrogen atom in groups such as an amine, a hydroxylamine, a nitrile, a nitro group, an N-oxide, a hydrazide, an azide, and an enamine; and other heteroatoms in various other groups.

The term “alkyl” refers to substituted and unsubstituted, straight-chain and branched alkyl groups and cycloalkyl groups having from 1 to 40 carbon atoms (C₁-C₄₀), 1 to 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. The term “alkyl” encompasses n-alkyl, isoalkyl, and anteisoalkyl groups as well as other branched forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” refers to substituted and unsubstituted, straight-chain and branched divalent alkenyl and cycloalkenyl groups having from 2 to 20 carbon atoms (C₂-C₂₀), 2 to 12 carbon atoms (C₂-C₁₂), 2 to 8 carbon atoms (C₂-C₈) or, in some embodiments, 2 to 4 carbon atoms (C₂-C₄) and at least one carbon-carbon double bond. Examples of straight-chain alkenyl groups include those with 2 to 8 carbon atoms, such as —CH═CH—, —CH═CHCH₂—, and the like. Examples of branched alkenyl groups include, but are not limited to, —CH═C(CH₃)— and the like.

The term “alkylene” refers to substituted and unsubstituted, straight-chain and branched divalent alkylene groups and cycloalkylene groups having from 1 to 40 carbon atoms (C₁-C₄₀), 1 to 20 carbon atoms (C₁-C₂₀), 1 to 12 carbons (C₁-C₁₂), 1 to 8 carbon atoms (C₁-C₈) or, in some embodiments, 1 to 4 carbon atoms (C₁-C₄), from 1 to 5 carbon atoms (C₁-C₅), 2 to 5 carbon atoms (C₂-C₅) or 3 to 4 carbon atoms (C₃-C₄). Examples of straight-chain alkylene groups include those with 1 to 8 carbon atoms such as methylene (—CH₂—), ethylene (—CH₂CH₂—), n-propylene (—CH₂CH₂CH₂—), n-butylene (—CH₂(CH₂)₂CH₂—) and the like. Examples of branched alkylene groups include, but are not limited to, isopropylidene (CH₂CH(CH₃)) and the like. Examples of cycloalkylene groups include, but are not limited to, cyclopropylidene, cyclobutylidene, cyclopentylidene and the like.

The term “alkynyl” refers to hydrocarbyl moieties of the scope of alkenyl but having one or more triple bonds. The alkynyl group can be substituted or unsubstituted.

The term “hydroxyalkyl” refers to alkyl groups as defined herein substituted with at least one hydroxyl (—OH) group. The hydroxyalkyl can be otherwise unsubstituted or substituted.

The term “cycloalkyl” refers to substituted and unsubstituted, cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. The term “cycloalkyl” may be used to refer to the substituent when it is attached to the remainder of the compound by an atom of the cyclo portion of the substituent. The term “alkylcyclyl” may be used to refer to the substituent when it is attached to the remainder of the compound by an atom of the alkyl portion of the substituent. The terms “cycloalkyl” and “alkylcyclyl” may be used interchangeably herein. Put another way, “cycloalkyl” may be used to refer to the substituent when it is attached to the remainder of the compound by an atom of the cyclo portion of the substituent or by an atom of the alkyl portion of the substituent.

The term “acyl” refers to substituted and unsubstituted groups containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroary lalkyl group or the like. In the special case wherein the carbonyl carbon atom is bonded to a hydrogen, the group is a “formyl” group, i.e., an acyl group. An acyl group can include 0 to about 12-40, 6-10, 1-5 or 2-5 additional carbon atoms bonded to the carbonyl group. An acryloyl group is an example of an acyl group. An acyl group can also include heteroatoms. A nicotinoyl group (pyridyl-3-carbonyl) is an example of an acyl group. Other examples include acetyl, benzoyl, phenylacetyl, pyridylacetyl, cinnamoyl, acryloyl groups, and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group. An example of a heterocyclylcarbonyl group is a prolyl group, wherein the prolyl group can be a D- or an L-prolyl group.

The term “aryl” refers to substituted and unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6 to 14 carbons (C₆-C₁₄) or 6 to 10 carbon atoms (C₆-C₁₀) in the ring portions of the groups. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups. An aryl can be mono-, bi-, or polycyclic.

The term “heteroaryl” refers to substituted and unsubstituted aromatic 3-12 membered ring structures, 5-12 membered ring structures, or 5-10 membered ring structures, in which a ring includes 1-4 heteroatoms. Heteroaryl groups include, for example, pyrrole, furan, thiophene, imidazole, oxazole, thiazole, triazole, pyrazole, pyridine, pyrazine, pyridazine, pyrimidine, and the like. A heteroaryl can be mono-, bi-, or polycyclic.

The terms “aralkyl” and “arylalkyl” refer to substituted and unsubstituted alkyl groups in which a hydrogen or carbon bond of an alkyl group is replaced with a bond to an aryl group. Representative aralkyl groups include, but are not limited to, benzyl and phenylethyl groups and fused (cycloalkylaryl) alkyl groups, such as 4-ethyl-indanyl. The terms “alkylaryl” and “arylalkyl” refer to an aryl substituted with an alkyl group. The term “alkylaryl” may be used to refer to the substituent when it is attached to the remainder of the compound by an atom on the alkyl portion of the substituent. The term “arylalkyl” may be used to refer to the substituent when it is attached to the remainder of the compound by an atom of the aryl portion of the substituent. The terms “alkylaryl” and “arylalkyl” may be used interchangeably herein. Put another way, “arylalkyl” may be used to refer to the substituent when it is attached to the remainder of the compound by an atom of the aryl portion of the substituent or by an atom of the alkyl portion of the substituent. The terms “aralkenyl” and “arylalkenyl” refer to alkenyl groups in which a hydrogen or carbon bond of an alkyl group is replaced with a bond to an aryl group.

The term “arylacyl” refers to an aryl group (an aromatic chain) attached to an acyl group (a group or radical of the form RCO— in which R is organic). The ary lacyl may be substituted or unsubstituted.

The term “arylalkylacyl” refers to an aryl group attached to an acyl group via an intervening alkyl group. The arylalkylacyl may be substituted or unsubstituted.

The term “heterocyclyl” refers to substituted and unsubstituted aromatic and non-aromatic ring compounds containing 3 or more ring members, of which one or more is a heteroatom such as, but not limited to, N, O, and S. Thus, a heterocyclyl can be a cycloheteroalkyl or a heteroaryl or, if polycyclic, any combination thereof. In some embodiments, heterocyclyl groups include 3 to 20 ring members, whereas other such groups have 3 to 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A “heteroaryl” group is an embodiment of a heterocyclyl group. The phrase “heterocyclyl group” includes fused ring species, including those that include fused aromatic and non-aromatic groups. Representative heterocyclyl groups include, but are not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heteroarylalkyl” refers to alkyl groups in which a hydrogen or carbon bond of an alkyl group is replaced with a bond to a heteroaryl group. The heteroarylalkyl can be substituted or unsubstituted.

The term “amine” refers to primary, secondary, and tertiary amines, which can be substituted or unsubstituted. Amines include, but are not limited to, R—NH₂, for example, alkylamines, arylamines, alkylarylamines; R₂NH wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions.

The term “amino group” refers to a substituent of the form —NH₂, —NHR, —NR₂, and —NR₃⁺, wherein each R is independently selected and includes protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, dialkylamino, and trialkylamino group. Such groups can be substituted or unsubstituted.

The term “aminoalkanoic acid” refers to a substituent that includes an amino group and a carboxylic acid group. The substituent can be substituted or unsubstituted.

The term “alkylamino” refers to a substituent that includes an alkyl group and an amino group. The substituent can be substituted or unsubstituted.

The term “alkylaminoalkyl” refers to a substituent that includes an amino group with an alkyl group replacing each of two hydrogens. The substituent can be substituted or unsubstituted.

The term “cyano” means —CN.

The terms “halo,” “halogen,” or “halide” group, by themselves or as part of another substituent, mean, unless otherwise stated, a fluorine, chlorine, bromine, or iodine atom.

The term “hydroxy” means —OH.

The term “haloalkyl” includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, perfluorobutyl, —CF(CH₃)₂ and the like. The haloalkyl can be substituted or unsubstituted.

The term “salts” and “pharmaceutically acceptable salts” refer to derivatives of the compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy benzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985.

The term “solvate” means a compound, or a salt thereof, that further includes a stoichiometric or non-stoichiometric amount of solvent bound by non-covalent intermolecular forces. Where the solvent is water, the solvate is a “hydrate”.

In view of the above, also provided is a a pharmaceutical composition comprising one or more of the above-described compounds, together with one or more pharmaceutically acceptable diluents, excipients, or carriers. The one or more compounds can be, and desirably are, cell-potent inhibitors for NNMT, bisubstrates for NNMT and its cofactor, and selective for NNMT over other closely related methyltransferases.

A “pharmaceutical composition” refers to a chemical or biological composition suitable for administration to a subject (e.g., mammal). Such compositions may be specifically formulated for administration via one or more of a number of routes including, but not limited to, buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutically acceptable diluent, excipient, or carrier” generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000.

A “pharmaceutically acceptable diluent, excipient, or carrier” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well-known in the art. Except insofar as any conventional media or agent is incompatible with the active compound, use thereof in the pharmaceutical compositions disclosed herein is contemplated. Supplementary active compounds can also be incorporated into the compositions.

Pharmaceutical compositions can be, and desirably are, sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion, and by the use of surfactants.

In many cases, it can be desirably, and even preferable, to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be formulated in a time-release formulation, for example in a composition that includes a slow-release polymer. The active compounds can be prepared with carriers that will protect the compound against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical composition can be orally administered as a capsule (hard or soft), tablet (film coated, enteric coated or uncoated), powder or granules (coated or uncoated) or liquid (solution or suspension). The formulations may be conveniently prepared by any of the methods well-known in the art. The pharmaceutical compositions can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

The compounds can be administered by a variety of dosage forms as known in the art. Any biologically acceptable dosage form known to persons of ordinary skill in the art, and combinations thereof, are contemplated. Examples of such dosage forms include, without limitation, chewable tablets, quick-dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds, which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate; binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments may vary according to factors such as the disease state, age, gender, weight, patient history, risk factors, predisposition to disease, administration route, and pre-existing treatment regime (e.g., possible interactions with other medications) of the individual. Dosage regimens may be adjusted to provide the optimum inhibition of NNMT. For example, a single bolus may be administered, several divided doses may be administered over time, or the dose may be proportionally reduced or increased as indicated by the exigencies of the situation.

“Dosage unit form,” as used herein, refers to physically discrete units suited as unitary dosages for the subject (e.g., a mammalian subject); each unit containing a predetermined quantity of active compound calculated to produce the desired level of NNMT inhibition in association with the required pharmaceutical carrier. The specification for the dosage unit forms are dictated by, and directly dependent on, the unique characteristics of the active compound and the limitations inherent in the art of compounding such an active compound for individuals with pre-existing sensitivities.

A dosage is typically administered once, twice, or thrice a day, although more frequent dosing intervals are possible. The dosage may be administered every day, every 2 days, every 3 days, every 4 days, every 5 days, every 6 days, and/or every 7 days (i.e., once a week). In one embodiment, the dosage may be administered daily for up to and including 30days, preferably between 7-10 days. In another embodiment, the dosage may be administered twice a day for 10 days. If the patient has a chronic disease or condition for which the inhibition of NNMT is beneficial, the dosage may be administered for as long as signs and/or symptoms persist. The patient may require “maintenance treatment” where the patient is receiving dosages every day for months, years, or the remainder of their lives. In addition, the composition may be administered to effect prophylaxis of recurring symptoms or signs. For example, the dosage may be administered once or twice a day to prevent the onset of symptoms in patients at risk, especially for asymptomatic patients.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect, i.e., the composition is applied directly where its action is desired. Administration can be enteral wherein the desired effect is systemic (non-local), i.e., the composition is given via the digestive tract. Administration can be parenteral, where the desired effect is systemic, i.e., the composition is given by other routes than the digestive tract.

Thus, in further view of the above, also provided is a method for inhibiting NNMT in a subject in need thereof. The method comprises administering to the subject an NNMT-inhibiting effective amount of an above-described compound or a pharmaceutical composition comprising same, together with one or more pharmaceutically acceptable diluents, excipients, or carriers. The compound can be, and desirably is, a cell-potent inhibitor for NNMT, a bisubstrate for NNMT and its cofactor, and selective for NNMT over other closely related methyltransferases. The subject can have cancer, such as, but not limited to, cervical, prostate, lung, breast (e.g., triple negative breast cancer), renal, colorectal, or pancreatic cancer, melanoma, glioblastoma, or neuroblastoma. The subject can have diabetes, a liver disease, scleroderma, or Parkinson's disease. In various embodiment of the methods, the compound can be administered simultaneously or sequentially, in either order, with one or more other active agents, such as one or more compound with the same or different mode of action.

The term “effective amount,” such as when used in the context of “an NNMT-inhibiting effective amount,” refers to that amount of one or more compounds that inhibits NNMT in a tissue system, animal or human, that is being sought by a researcher, veterinarian, medical doctor or other clinician. The inhibition of NNMT can include alleviation of the signs and symptoms of a disease, such as cancer (e.g., cervical, prostate, lung, breast (e.g., triple-negative breast cancer), colorectal, renal, or pancreatic cancer, melanoma, glioblastoma or neuroblastoma), diabetes, liver disease, scleroderma, or Parkinson's disease. In some embodiments, the effective amount is that which inhibit NNMT, including the alleviation of signs and symptoms of a disease, at a reasonable benefit/risk ratio applicable to any medical treatment. However, the total daily usage of the compounds and compositions may be decided by the attending veterinarian, medical doctor or other clinician physician within the scope of sound medical judgment. The specific effective dose level for any particular subject will depend upon a variety of factors, including a pre-existing disease or disorder and the severity thereof; the activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well-known to the researcher, veterinarian, medical doctor or other clinician. The effective amount can be selected with reference to any toxicity, or other undesirable side effect, that might occur during administration of one or more of the compounds.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to, the species of mammal, including human, its size, age, and general health, the specific disease or disorder involved, the degree of or involvement or the severity of the disease or disorder, the response of the individual subject, the particular compound administered, the mode of administration, the bioavailability characteristics of the preparation administered, the dose regimen selected, the use of concomitant medication, and other relevant circumstances.

The term “subject” includes human and non-human animals such as companion animals (dogs and cats and the like) and livestock animals. Livestock animals are animals raised for food production. The subject to be treated is preferably a mammal, in particular a human.

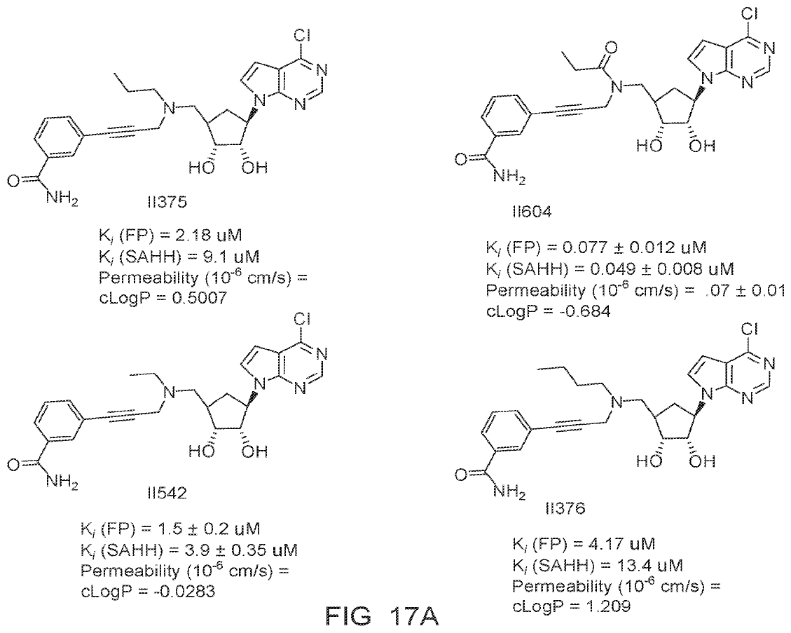

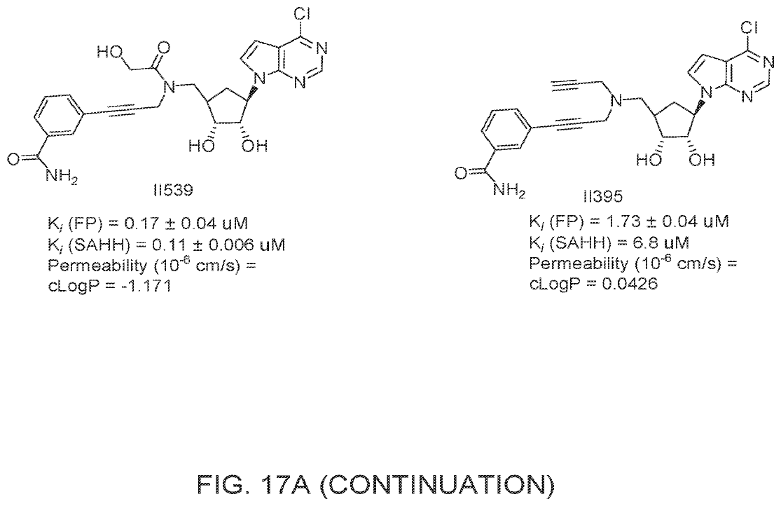

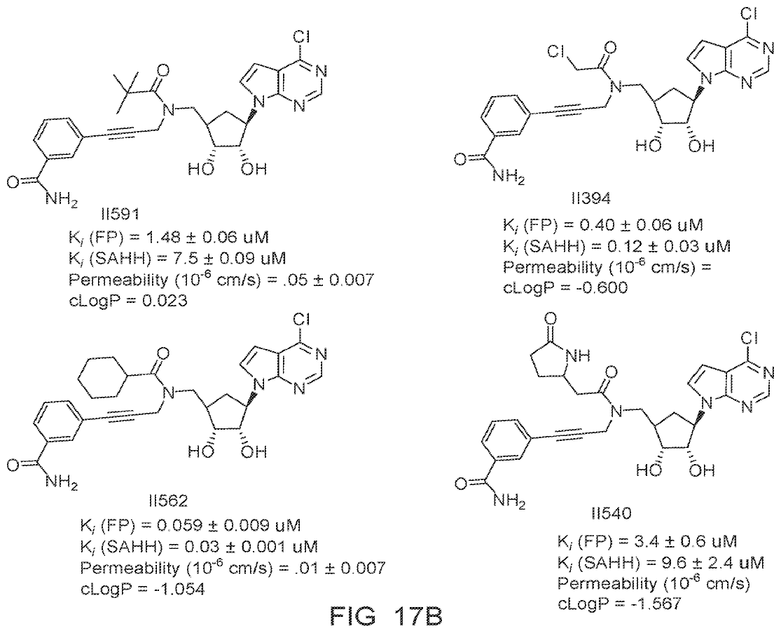

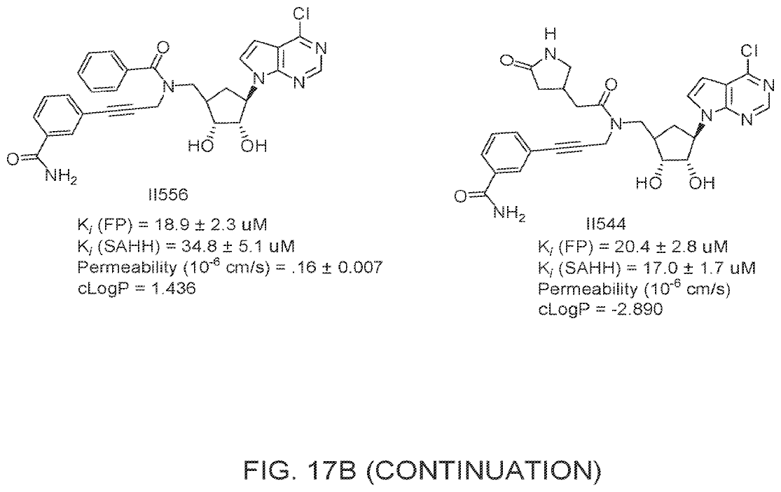

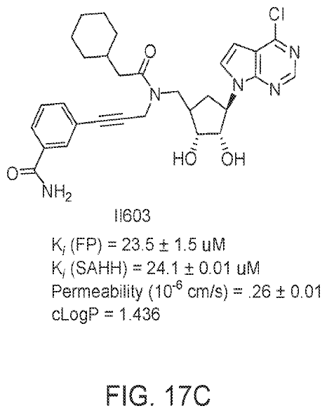

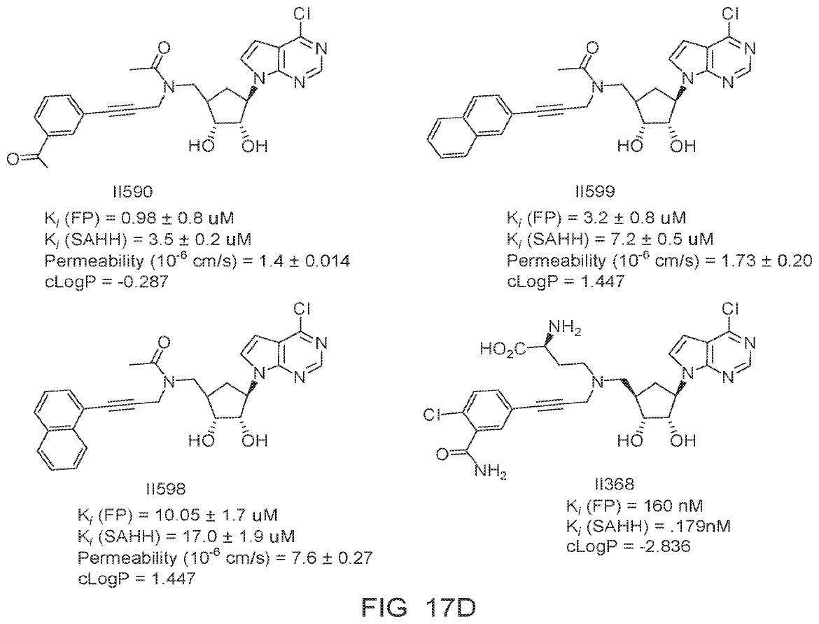

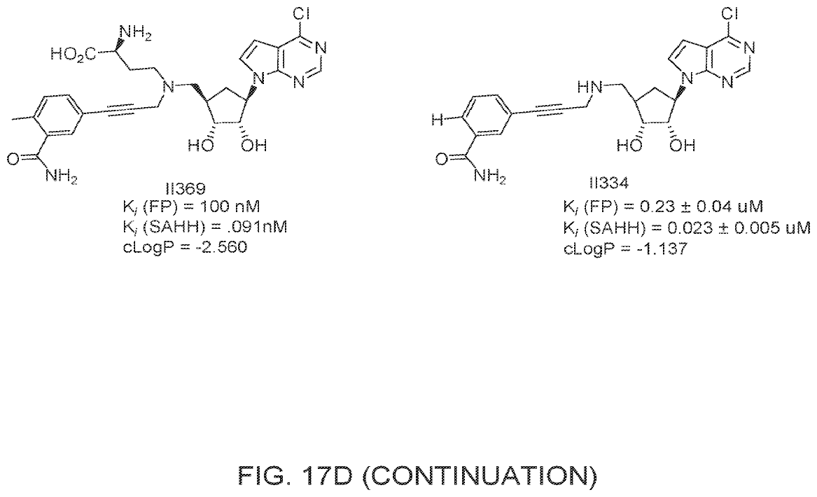

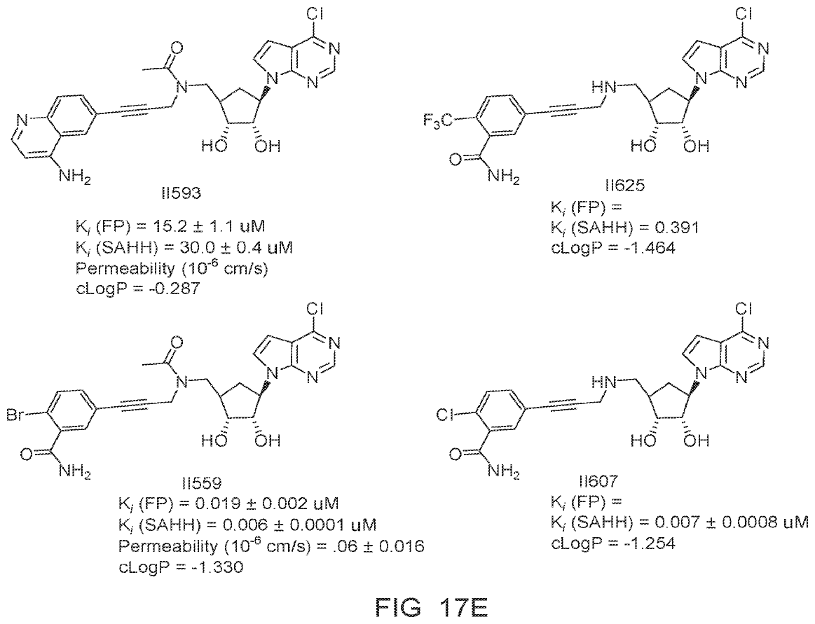

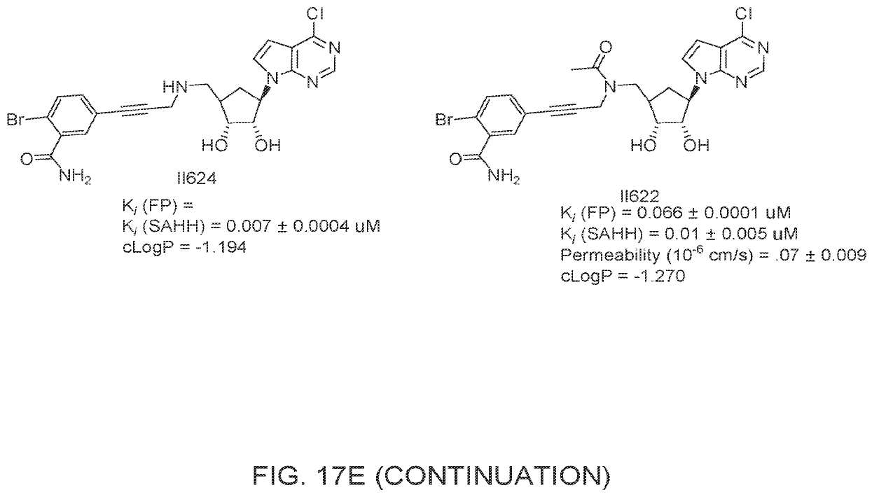

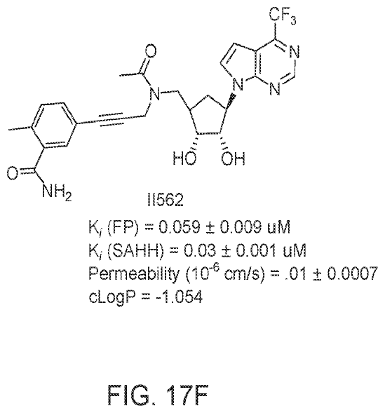

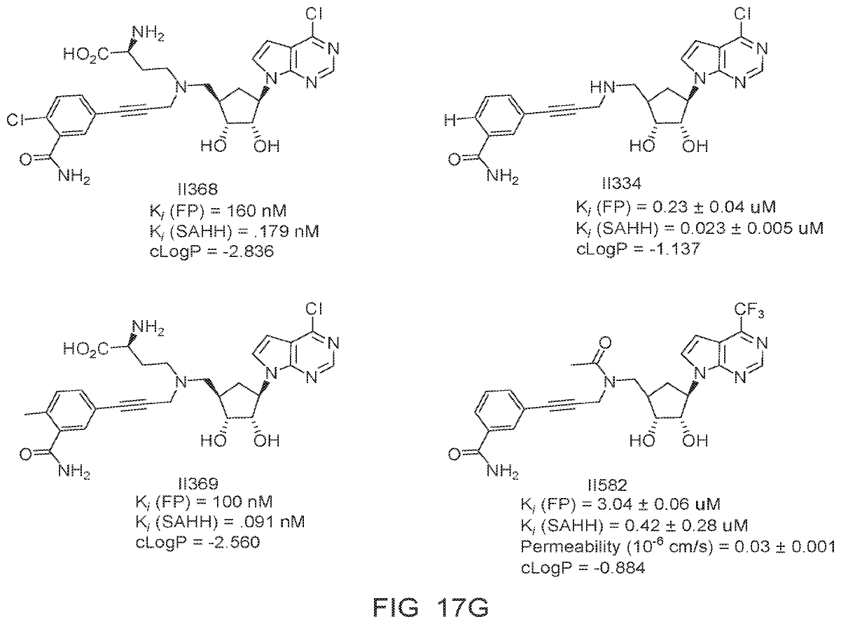

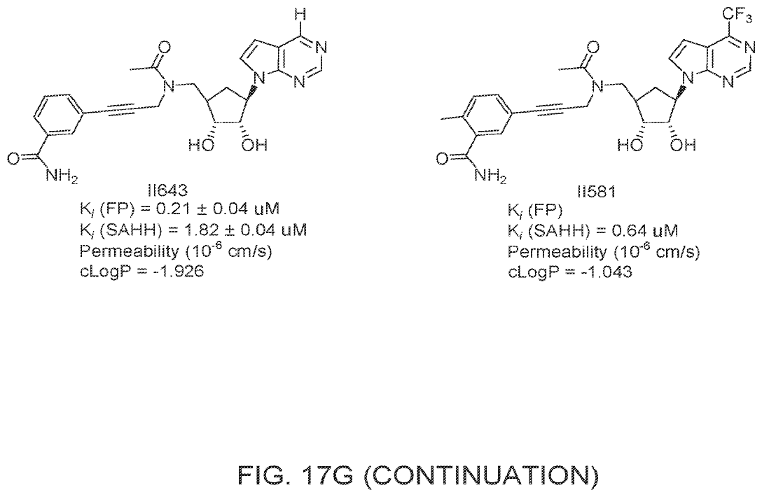

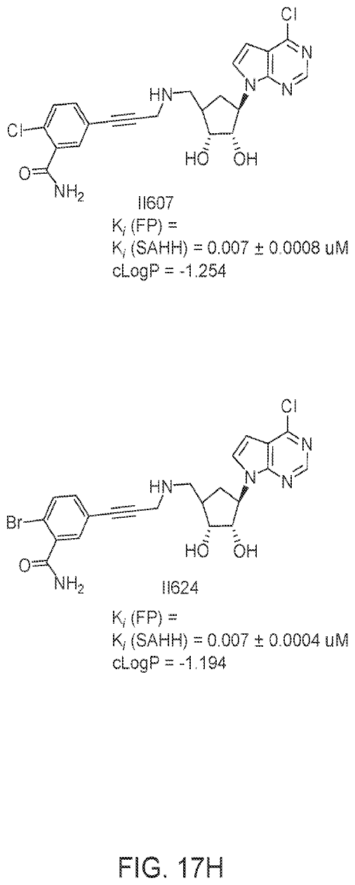

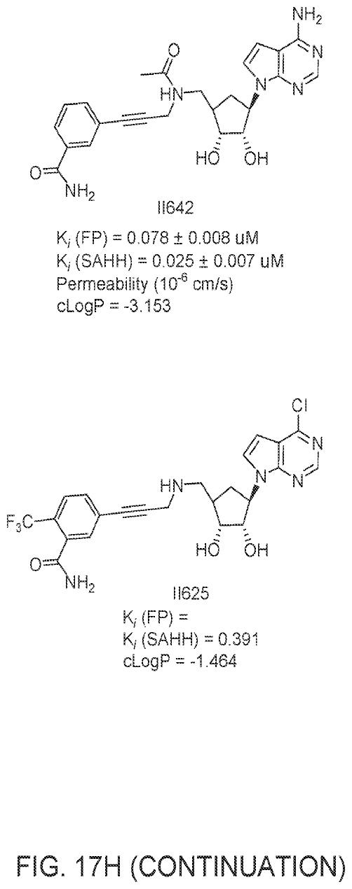

Also provided is a compound as shown in any one of FIGS. 17A-17H or a pharmaceutical composition comprising same, together with one or more pharmaceutically acceptable diluents, excipients, or carriers. The compound can be, and desirably is, a cell-potent inhibitor for NNMT, a bisubstrate for NNMT and its cofactor, and selective for NNMT over other closely related methyltransferases.

In view of the above, also provided is a method for inhibiting nicotinamide N-methyltransferase (NNMT) in a subject in need thereof. The method comprises administering to the subject an NNMT-inhibiting effective amount of the aforementioned compound (FIGS. 17A-17H) or a pharmaceutical composition comprising same. The subject can have cancer, such as, but not limited to, cervical, prostate, lung, breast (e.g., triple negative breast cancer), colorectal, renal, or pancreatic cancer, melanoma, glioblastoma, or neuroblastoma. The subject can have diabetes, a liver disease, scleroderma, or Parkinson's disease. In various embodiment of the methods, the compound can be administered simultaneously or sequentially, in either order, with one or more other active agents, such as one or more compound with the same or different mode of action.

### EXAMPLES

The following examples serve to illustrate the present disclosure. The examples are not intended to limit the scope of the claimed invention in any way.

### Example 1 Design

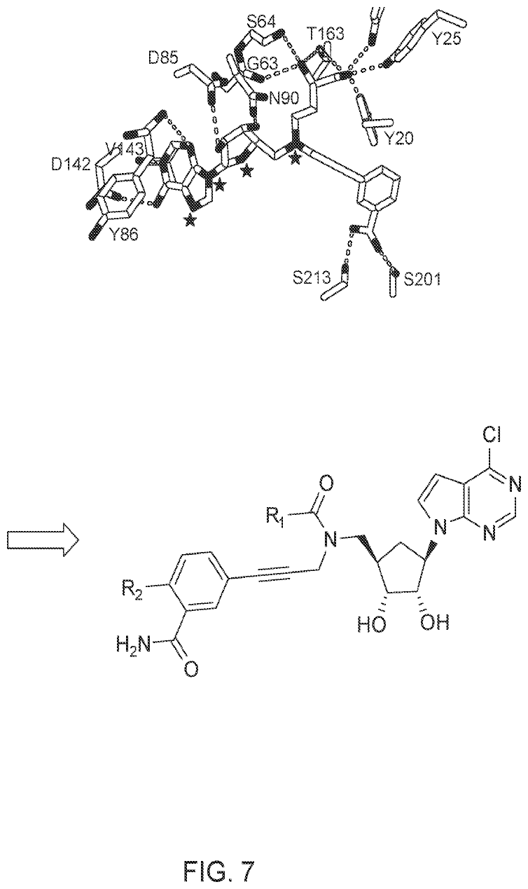

LL320 was chosen as a lead compound because of its high potency and selectivity. To boost the cellular potency of LL320, the heteroatom(s) that did not contribute to the binding to NNMT was/were eliminated to increase hydrophobicity. Examing the co-crystal structure of NNMT-LL320 (PDB ID: 6PVS), four heteroatoms (3Ns and 1O), which did not display any direct interaction with NNMT, were identified (FIG. 7). Considering synthetic tractability, two heteroatoms (N7 in adenine and the O in ribose) were replaced with carbon atoms (FIG. 7). The amino group on N⁶ was replaced with chloride to introduce potential halo bonding to retain the interaction with NNMT with increased lipophilicity. Halo bonding has been shown to improve binding affinity and selectivity, for example, in the inhibition of aldose reductase with IDD 594, where a bromine substitution resulted in about 1,000-fold selectivity (Hong et al., Biochem 57: 5775-5779(2018); Palanichamy et al., Clin Cancer Res 23: 2325-2334 (2017); and Jung et al., J Clin Invest 2: 1-23 (2017)). The possible replacement of the aspartic acid moiety and the benzamide with other functional groups to increase the hydrophobicity was also explored (FIG. 7).

### Example 2 Synthesis

The reagents and solvents were purchased from commercial sources (Fisher and Sigma-Aldrich) and used directly. Analytical thin-layer chromatography (TLC) was performed on ready-to-use plates with silica gel 60 (Merck, F254). Flash column chromatography was performed over silica gel (grade 60, 230-400 mesh) on a Teledyne Isco CombiFlash purification system. Final compounds were purified by preparative reversed-phase high-pressure liquid chromatography (RP-HPLC) that was performed on an Agilent 1260 Series system. The Agilent 1260 Infinity II Variable Wavelength Detector (G7114A, UV=254 nM) and a WatersBEHC18 (130 Å, 5 μm, 10 mm×250 mm), at a flow rate of 4 mL/min using a solvent system of 100% water with 0.1% TFA to 50% methanol over 30 minutes, were used to purify the final compounds. NMR spectra were acquired on a Bruker AV500 instrument (500 MHz for ¹H NMR, 126 MHz for ¹³C NMR). TLC-MS was carried out using an Advion CMS-L MS. Matrix-assisted laser desorption ionization mass spectrometry (MALDI-MS) data were acquired in the positive-ion mode using a Sciex 4800 MALDI TOF/TOF MS. The Agilent 1260 Infinity II Variable Wavelength Detector (G7114A, UV=254 nM) and an Agilent ZORBAX RR SB-C18 (80 Å, 3.5 μm, 4.6×150 mm), at a flow rate of 1 mL/min using a solvent system of 100% water with 0.1% TFA to 40 or 60% methanol over 20 minutes, were used to assess the purity of final compounds. All the purity of target compounds showed >95% in RP-HPLC. Agilent 6470 QQQ coupled to an Agilent 1290 UPLC system with an Imtakt Intrada Amino acid column (2.0×150 mm) was used to analyze the metabolite in the cellular methylation studies. Mobile phase A was composed of 100% CH₃CN+0.3% formic acid, and mobile phase B was composed of 20:80 (v/v): CH₃CN/100 mM ammonium formate in water. The flow rate started at 0.3 mL/min. The gradient started with 0% B and increased linearly to 100% B over 11 minutes with a flow rate of 0.3 ml/min, followed by an isocratic gradient of 100% B for 2 minutes at 0.4 ml/min. Then, the column was equilibrated with 0% B for 7 minutes. All the purity of target compounds showed >95% purity.

Commercially available aldehyde was protected with triethoxymethane to afford 1, which was coupled with (1S,2R,3S,5S)-3-amino-5-(hydroxymethyl)cyclopentane-1,2-diol to yield compound 2. After protection of the diol, the primary alcohol was oxidized to the aldehyde using Dess-Martin periodinane (DMP). After oxidation, the aldehyde reacted with 3-(3-aminoprop-1-yn-1-yl) benzamide to yield the secondary amine 4 through reductive amination. Coupling reaction of 4 with different aliphatic and aromatic acid chlorides followed by subsequent acid-catalyzed deprotection produced the final bisubstrate analogues 5a-5k (Scheme 1a).

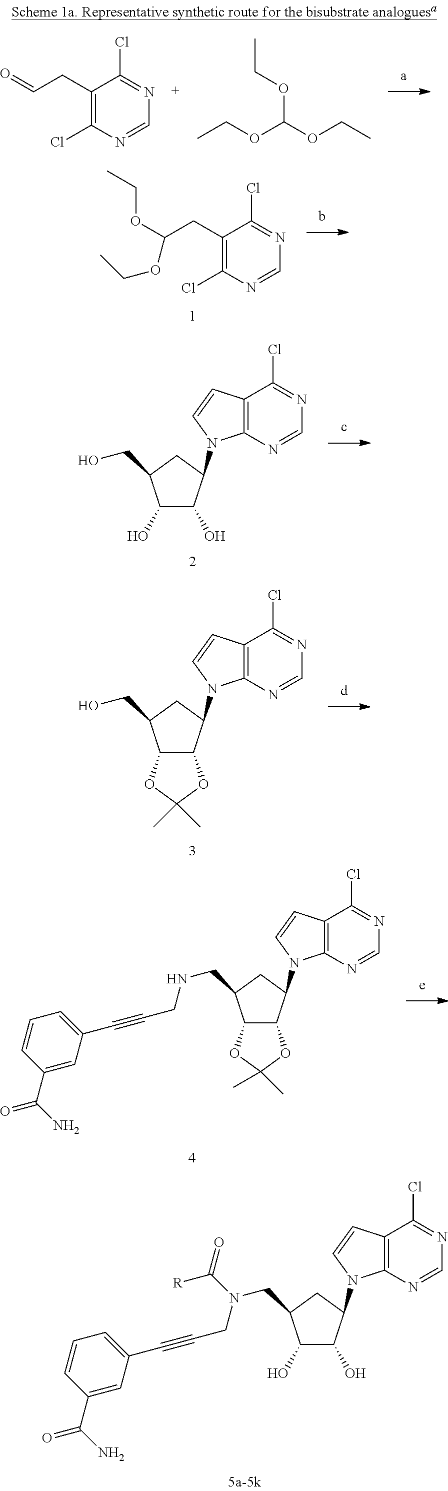

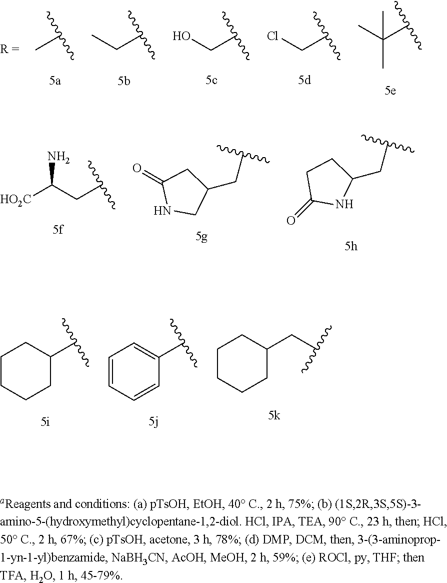

General procedure A in Scheme 1a (Sonogoshira coupling). To a solution of iodide (0.06 mmol) in 0.5 mL toluene/DMF was added the alkyne (0.08 mmol), DIPEA, and CuI. The reaction mixture was degassed before the addition of Pd(Ph₃)₄. The reaction mixture was heated at 70° C. for 3 hours. The reaction mixture was cooled to room temperature, filtered through a pad of celite, and used without further purification.

4,6-dichloro-5-(2,2-diethoxyethyl) (1). Compound 1 was synthesized as previously reported. A mixture of 2-(4,6-dichloropyrimidin-5-yl) acetaldehyde (5 g, 26.1 mmol), triethoxymethane (4.65 g, 1.2 mmol), and Ts—OH (298.7 mg, 0.06 mmol) in EtOH (30 mL) was stirred at 40° C. for 2 hours. After completion of the reaction, aqueous Na₂CO₃ was added to the mixture to adjust pH to 8. The solvent was removed under reduced pressure and the residue extracted with EtOAc (3×), washed with water (10 ml). The organic layer was dried over anhydrous Na₂SO₄, filtered, and concentrated under reduced pressure to give the residue that was purified by column chromatography on silica gel to afford compound 1 (5.2 g, 75%) as a clear oil. ¹H NMR (500 MHz, Chloroform-d) δ 8.65 (s, 1H), 4.82 (t, J=5.7 Hz, 1H), 3.77-3.68 (m, 2H), 3.51-3.42 (m, 2H), 3.27 (d, J=5.8 Hz, 2H), 1.15 (t, J=7.0 Hz, 6H). ¹³C NMR (126 MHz, CDCl₃) δ 162.85, 155.99, 129.35, 101.06, 63.06, 35.52, 15.32. ESI-MS (m/z) calculated for C₁₀H₁₅Cl₂N₂O₂ [M+H]+: 265.05, found 265.0.

General procedure B in Scheme 1a (Aldehyde protection). A mixture of aldehyde (1 mmol), triethoxymethane (1.2 mmol), and Ts—OH (0.06 mmol) in EtOH (10 mL) was stirred at 40° C. for 2 hours. After completion of the reaction, aqueous Na₂CO₃ was added to the mixture to adjust pH to 8. The solvent was removed under reduced pressure, and the residue was extracted with EtOAc (3×) and washed with water (10 ml). The organic layer was dried over anhydrous Na₂SO₄, filtered, and concentrated under reduced pressure to give the residue that was purified by column chromatography on silica gel to afford the desired product.

(1R,2S,3R,5R)-3-(4-chloro-7H-pyrrolo[2,3-d]pyrimidin-7-yl)-5-(hydroxymethyl)cyclopentane-1,2-diol (2). Compound 2 was synthesized following an earlier reported procedure (Jin et al., Adv Exp Med Biol 754: 3-29 (2013)). To a mixture of 1 (600 mg, 2.30 mmol) and (1S,2R,3S,5S)-3-amino-5-(hydroxymethyl)cyclopentane-1,2-diol. HCl (464 mg, 2.53 mmol) in IPA: H₂O (7:1) 10 mL was added TEA (800 μL, 5.74 mmol) in one portion at room temperature under N₂. The reaction was heated to 90° C. and stirred for 23 hours. The mixture was cooled to 50° C. and 4 M HCl (1.15 mL, 4.59 mmol) was added slowly. The reaction was then stirred at 50° C. for 2 hours. The reaction was cooled to room temperature and NaHCO₃ was added slowly. The reaction mixture was extracted with EA, dried with Na₂SO₄, filtered, and purified by flash column eluting with 8% MeOH in DCM to afford compound 2 (430 mg, 67%) as a yellow solid. ¹H NMR (500 MHz, Methanol-d₄) δ 8.53 (s, 1H), 7.71 (d, J=3.7 Hz, 1H), 6.66 (d, J=3.7 Hz, 1H), 5.17-5.09 (m, 1H), 4.45-4.38 (m, 1H), 4.08-4.01 (m, 1H), 3.70 (d, J=5.8 Hz, 2H), 2.45-2.36 (m, 1H), 2.29-2.20 (m, 1H), 1.91-1.77 (m, 1H). ¹³C NMR (126 MHz, MeOD) δ 152.69, 152.47, 150.89, 130.06, 119.16, 100.37, 77.05, 73.74, 64.52, 61.74, 46.72, 30.40. ESI-MS (m/z) calculated for C₁₂H₁₅ClN₃O₃ [M+H]+: 284.07, found 284.0.

General procedure C in Scheme 1a (Hydroxyl protection). To a solution of nitrile (0.06 mmol) in 0.5 mL DMSO was added K₂CO₃. The reaction mixture was cooled to 0° C. before the addition of hydrogen peroxide. The reaction mixture was warmed to room temperature and stirring was continued for 3 hours. The reaction was quenched with saturated NaHCO₃ and extracted with EA (3×). The combined organic layers were dried over Na₂SO₄, filtered. and concentrated. The residue was purified with a silica gel column to provide the desired product.

((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)methanol (3). To a mixture of compound 2 (850) mg, 3.0 mmol) in acetone (200 mL) was added p-TsOH (5.7 g, 30 mmol). The reaction was stirred at room temperature for 3 hours. The reaction mixture was quenched on ice with cold NaHCO₃, extracted with EA and washed with brine. The combined organic layer was dried with Na₂SO₄, concentrated and purified in a flash column eluting with 60% EA in hexanes to afford 3 (760 mg, 78%) as a light-yellow solid. ¹H NMR (500 MHz, Chloroform-d) δ 8.55 (s, 1H), 7.33 (d, J=3.7 Hz, 1H), 6.56 (d, J=3.6 Hz, 1H), 5.03-4.95 (m, 1H), 4.92-4.86 (m, 1H), 4.69-4.62 (m, 1H), 3.84-3.70 (m, 2H), 3.19 (s, 1H), 2.46-2.36 (m, 2H), 2.33-2.21 (m, 1H), 1.53 (s, 3H), 1.25 (s, 3H). ¹³C NMR (126 MHz, CDCl₃) δ 152.11, 150.85, 150.29, 128.09, 118.21, 113.65, 99.96, 84.36, 81.51, 63.32, 62.41, 45.55, 33.65, 27.55, 25.13. ESI-MS (m/z) calculated for C₁₅H₁₉ClN₃O₃ [M+H]+: 324.11. found 324.2.

General procedure D in Scheme 1a (Reductive amination). To a solution of amine (0.06 mmol) in 0.5 mL MeOH was added the aldehyde (0.08 mmol) followed by 2 drops of AcOH. The resulting mixture was stirred for 30 minutes before NaBH₃CN (0.1 mmol) was added. After stirring for 2 hours at room temperature, the reaction was quenched with saturated NaHCO₃ and extracted with DCM (3×). The combined organic layers were dried over Na₂SO₄, filtered, and concentrated. The residue was purified with silica gel column to provide the desired product.

3-(3-((((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4 -yl)methyl)amino)prop-1-yn-1-yl)benzamide (4). To a mixture of 3 (570) mg, 1.76 mmol) in DCM (10 mL) was added DMP (1.49 g, 3.52 mmol) in one portion at 0° C. under N₂. The mixture was stirred at 0° C. for 3 hours. The reaction mixture was quenched with Na₂S₂O₃ (2× mass of alcohol) in saturated NaHCO₃ and stirred for 10 minutes. The reaction mixture was extracted with DCM, and the organic layer was washed twice with brine and dried with Na₂SO₄ to afford the crude aldehyde, which was used without any further purification.

To a solution of the crude aldehyde (100 mg, 0.3 mmol) and 3-(3-aminoprop-1-yn-1-yl)benzamide (65.0 mg, 0.37 mmol) in MeOH (2 mL) was added catalytic AcOH, and the mixture was stirred at room temperature for 30 minutess before adding NaCNBH₃ (29.3 mg, 0.46 mmol), after which the reaction was further stirred for 2 hours. After completion, the reaction mixture was diluted with MeOH, concentrated. and purified by flash column to afford 4 (108 mg, 59%). ¹H NMR (500 MHz, Methanol-d₄) δ 8.57 (s, 1H), 8.50 (s, 1H), 7.95 (t, J=1.8 Hz, 1H), 7.86-7.81 (m, 1H), 7.73 (d, J=3.7 Hz, 1H), 7.63-7.58 (m, 1H), 7.45 (t, J=7.8 Hz, 1H), 6.70 (d, J=3.7 Hz, 1H), 5.21-5.12 (m, 1H), 5.07-4.98 (m, 1H), 4.67-4.60 (m, 1H), 3.77 (s, 2H), 3.10-3.04 (m, 1H), 3.00-2.93 (m, 1H), 2.56-2.40 (m, 2H), 2.30-2.17 (m, 1H), 1.56 (s, 3H), 1.31 (s, 3H). ¹³C NMR (126 MHz, MeOD) δ 167.63, 151.38, 150.96, 149.77, 134.31, 134.05, 130.50, 128.81, 128.37, 127.07, 123.29, 117.80, 113.78, 99.30, 84.06, 83.17, 82.91, 61.36, 50.73, 43.34, 37.84, 35.48, 26.39, 23.99. ESI-MS (m/z) calculated for C₂₅H₂₇ClN₅O₃ [M+H]+: 480.17, found 480.1.

General procedure E in Scheme 1a (Deprotection of bisubstrate inhibitors). The protected inhibitor was stirred in a mixture of TFA:H₂O (4:1) for 1 hour. The resulting solution was concentrated, dissolved in methanol, and purified by reverse HPLC to get the corresponding bisubstrate compound.

3-(3-(N-(((1R,2R,3S,4R)-4-(4-chloro-7H-pyrrolo[2,3-d]pyrimidin-7-yl)-2,3-dihydroxycyclopentyl)methyl)acetamido)prop-1-yn-1-yl)benzamide (II399; 5a). To a solution of 4 (60) mg, 0.13 mmol) and pyridine (20 mg, 0.25 mmol) in THF (2 mL) was added acetic anhydride (26 mg, 0.25 mmol). The reaction was stirred at room temperature for 1 hour, concentrated, and immediately treated with TFA:H₂O (1:1) for 2 hours and purified by prep HPLC to afford compound II399 (40 mg, 69%) as a mixture of rotamers. ¹H NMR (500 MHz, Methanol-d₄) δ 8.52 (d, J=2.7 Hz, 1H), 7.97-7.91 (m, 1H), 7.88-7.80 (m, 1H), 7.73-7.65 (m, 1H), 7.62-7.54 (m, 1H), 7.46-7.38 (m, 1H), 6.66 (t, J=3.7 Hz, 1H), 5.14-4.97 (m, 1H), 4.61-4.41 (m, 3H), 4.09-3.88 (m, 1H), 3.82-3.75 (m, 1H), 3.74-3.65 (m, 1H), 2.64-2.39 (m, 2H), 2.26 (d, J=12.4 Hz, 3H), 1.99-1.79 (m, 1H). ¹³C NMR (126 MHz, MeOD) δ 174.06, 152.65, 152.53, 150.95, 135.75, 135.47, 131.90, 130.95, 130.28, 129.85, 129.75, 128.86, 128.60, 124.03, 119.23, 100.41, 100.28, 86.01, 84.53, 76.65, 76.14, 74.50, 74.30, 63.13, 62.05, 52.54, 50.16, 44.06, 43.17, 40.26, 36.46, 31.90, 21.77. HRMS (m/z) calculated for C₂₄H₂₅ClN₅O₄ [M+H]+: 482.1589, found 482.1588.

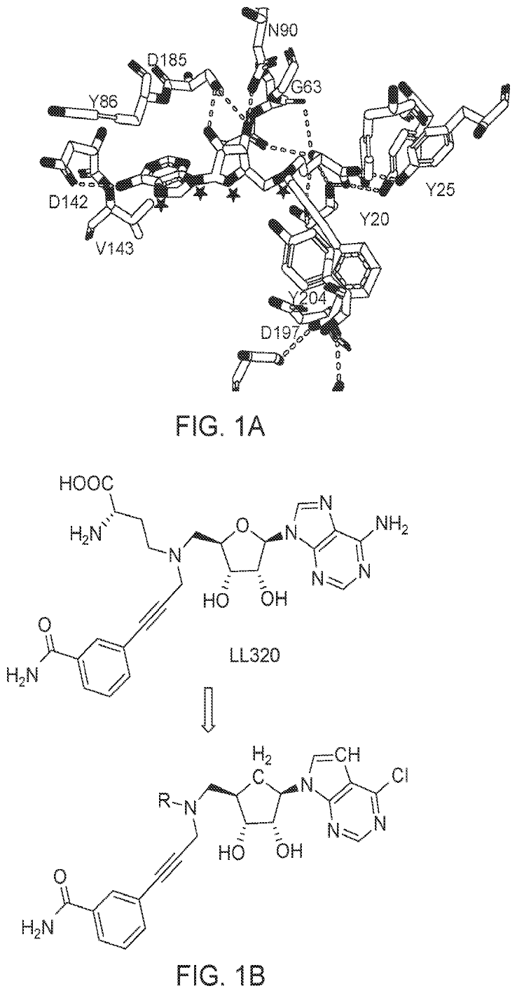

To build potent bisubstrate analogs, it is essential to mimic the structure of SAM to retain the interaction with methyltransferases. Nevertheless, it is also important to increase the lipophilicity of the SAM mimic to boost cellular potency without perturbing its interactions with the target. After close examination of the NNMT-LL320co-crystal structure (PDB ID: 6PVS), 4 heteroatoms were identified in LL320 that did not display any direct interaction with NNMT (FIG. 1A) (Chen et al., J. Med. Chem. 62: 10783-10797 (2019)).

The N⁷ in adenine and the O atom in the ribose ring were replaced with two carbon atoms regarding the synthetic tractability. Secondly, the amino group on N⁶ was substituted with chloride to create a potential halogen bond with NNMT to retain the interaction with increased lipophilicity (Mondal et al., Angew. Chemie-Int. Ed. 58: 12476-12480 (2019); and Auffinger et al., Proc. Natl. Acad. Sci. USA 101: 16789-16794 (2004)).

The aspartic moiety of SAM is a double-edged sword as it is known to form multiple interactions with NNMT but also to impede cell permeability. Thus, replacement of the aspartic acid with a less polar moiety was attempted to boost the lipophilicity. The synthesis started from commercially available aldehyde (Scheme 1b). Protection with triethoxymethane yielded 1, which was then coupled with (1S,2R,3S,5S)-3-amino-5-(hydroxymethyl)cyclopentane-1,2-diol to generate compound 2 (Zhang et al., Chem Heterocycl Comp 54: 638-642 (2018)). After the diol was protected, the primary alcohol was oxidized to the aldehyde by Dess-Martin periodinane (DMP). Subsequent reductive amination with 3-(3-aminoprop-1-yn-1-yl)benzamide yielded the secondary amine 4, which was either reacted with acetic anhydride or iodoethane followed by acid-catalyzed deprotection to generate bisubstrate analogs II399 and II542 (Chen et al., J. Med. Chem. 62: 10783-10797 (2019)). The chloride was then substituted with an amino group to afford II642 by heating in ammonium hydroxide (Lambertucci et al., Bioorg. & Med. Chem. 17: 2812-2822 (2009)). As a control compound, an acetamide analog of LL320 was also prepared to generate II661.

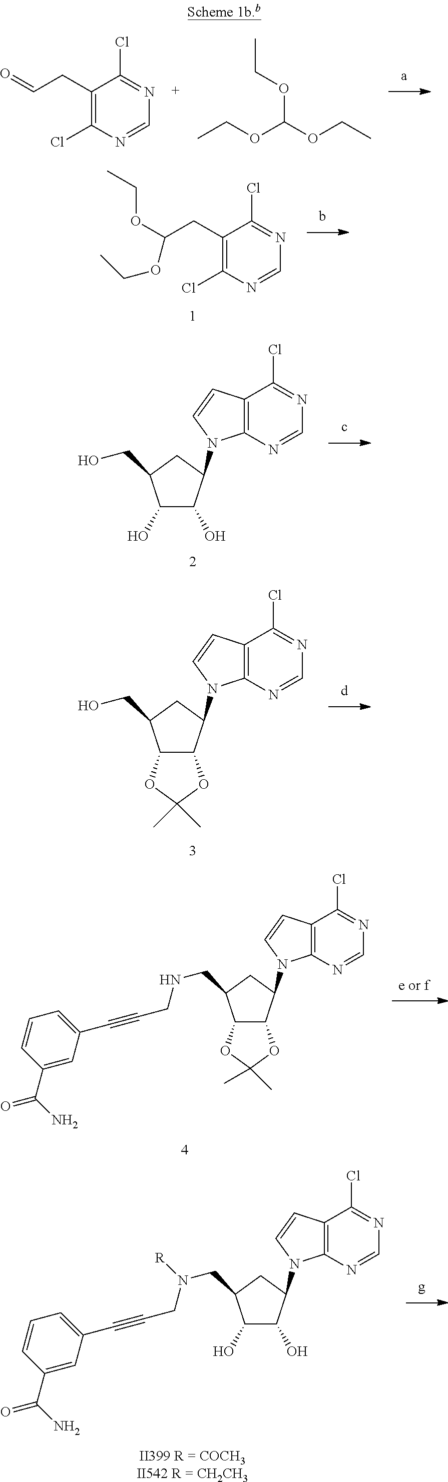

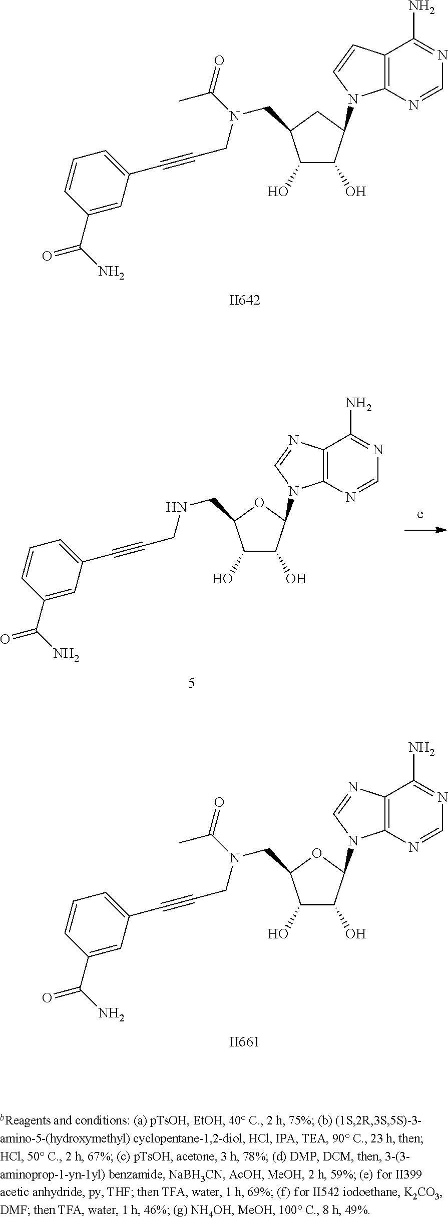 [SMILES: CC(=O)N(CC#Cc1cccc(C(N)=O)c1)C[C@H]1C[C@@H](n2ccc3c(N)ncnc32)[C@H](O)[C@@H]1O ; "II642" ; NC(=O)c1cccc(C#CCNC[C@H]2O[C@@H](n3cnc4c(N)ncnc43)[C@H](O)[C@@H]2O)c1 ; CC(=O)N(CC#Cc1cccc(C(N)=O)c1)C[C@H]1O[C@@H](n2cnc3c(N)ncnc32)[C@H](O)[C@@H]1O ; "II661"]

General procedure A in Scheme 1b (Aldehyde protection). Zhang et al., Chem Heterocycl Comp 54: 638-642 (2018) A mixture of aldehyde (1 mmol), triethoxymethane (1.2 mmol), and Ts—OH (0.06 mmol) in EtOH (10 mL) was stirred at 40° C. for 2 hours. After completion of the reaction, aqueous Na₂CO₃ was added to the mixture to adjust pH to 8. The solvent was removed under reduced pressure and the residue was extracted with EtOAc (3×) and washed with water (10 mL). The organic layer was dried over anhydrous Na₂SO₄, filtered, and concentrated under reduced pressure to give the residue that was purified by column chromatography on silica gel to afford the desired product.

4,6-dichloro-5-(2,2-diethoxyethyl) (1). Compound 1 was prepared according to the general procedure A. ¹H NMR (500 MHz, Chloroform-d) δ 8.65 (s, 1H), 4.82 (t, J=5.7 Hz, 1H), 3.77-3.68 (m, 2H), 3.51-3.42 (m, 2H), 3.27 (d, J=5.8 Hz, 2H), 1.15 (t, J=7.0 Hz, 6H). ¹³C NMR (126 MHz, CDCl₃) δ 162.85, 155.99, 129.35, 101.06, 63.06, 35.52. 15.32. ESI-MS (m/z) calculated for C₁₀H₁₅Cl₂N₂O₂ [M+H]+: 265.05, found 265.0. General procedure B in Scheme 1b (Hydroxyl protection) (Chen et al., J. Med. Chem. 62: 10783-10797 (2019)). To a solution of alcohol in acetone was added Ts—OH (10 equ.). The reaction mixture was stirred at room temperature for 3 hours. After completion. the reaction was quenched with cold saturated NaHCO₃ and extracted with EA (3×). The combined organic layers were dried over Na₂SO₄, filtered, and concentrated. The residue was purified with a silica gel column to provide the desired product.

(1R,2S,3R,5R)-3-(4-chloro-7H-pyrrolo[2,3-d]pyrimidin-7-yl)-5-(hydroxymethyl)cyclopentane-1,2-diol (2). Compound 2 was synthesized following an earlier reported procedure (Berthelot et al., Int'l Pat App Pub No. WO/2017/153186). To a mixture of 1 (1 g, 3.7 mmol) and (1S,2R,3S,5S)-3-amino-5-(hydroxymethyl)cyclopentane-1,2-diol. HCl (692 mg, 3.7 mmol) in IPA: H₂O (7:1) 12 mL was added TEA (1.3 mL, 9.4 mmol) in one portion at rt under N₂. The reaction was heated to 90° C. and stirred for 23 hours. The mixture was cooled to 50° C. and 4 M HCl (1.8 mL, 7.5 mmol) was added slowly. The reaction was then stirred at 50° C. for 2 hours. The reaction was cooled to rt and NaHCO₃ was added slowly. The reaction mixture was extracted with EA, dried with Na₂SO₄, filtered and purified by flash column eluting with 8% MeOH in DCM to afford compound 2 as a yellow solid. ¹H NMR (500 MHz, Methanol-d₄) δ 8.53 (s, 1H), 7.71 (d, J=3.7 Hz, 1H), 6.66 (d, J=3.7 Hz, 1H), 5.17-5.09 (m, 1H), 4.45-4.38 (m, 1H), 4.08-4.01 (m, 1H), 3.70 (d, J=5.8 Hz, 2H), 2.45-2.36(m, 1H), 2.29-2.20 (m, 1H), 1.91-1.77 (m, 1H). ¹³C NMR (126 MHz, MeOD) δ 152.69, 152.47, 150.89, 130.06, 119.16, 100.37, 77.05, 73.74, 64.52, 61.74, 46.72, 30.40. ESI-MS (m/z) calculated for C₁₂H₁₅ClN₃O₃ [M+H]⁺: 284.07, found 284.0.

General procedure C in Scheme 1b (Sonogoshira coupling) (Policarpo et al., J Med Chem 62(21): 9837-9873 (2019)). To a solution of iodide (0.06 mmol) in 0.5mL toluene/DMF were added the alkyne (0.08 mmol), DIPEA, and CuI. The reaction mixture was degassed before the addition of Pd(Ph₃)₄. The reaction mixture was heated at 70° C. for 3 hours. The reaction mixture was cooled to room temperature, filtered through a pad of celite, and stirred in a mixture of TFA: H₂O (4:1) for 1 hour. The resulting solution was concentrated and dissolved.

((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)methanol (3). Compound 3 was prepared according to the general procedure B. ¹H NMR (500 MHz, Chloroform-d) δ 8.55 (s, 1H), 7.33 (d, J=3.7 Hz, 1H), 6.56 (d, J=3.6 Hz, 1H), 5.03-4.95 (m, 1H), 4.92-4.86 (m, 1H), 4.69-4.62 (m, 1H), 3.84-3.70 (m, 2H), 3.19 (s, 1H), 2.46-2.36 (m, 2H), 2.33-2.21 (m, 1H), 1.53 (s, 3H), 1.25 (s, 3H). ¹³C NMR (126 MHz, CDCl₃) δ 152.11, 150.85, 150.29, 128.09, 118.21, 113.65, 99.96, 84.36, 81.51, 63.32, 62.41, 45.55, 33.65, 27.55, 25.13. ESI-MS (m/z) calculated for C₁₅H₁₉ClN₃O₃ [M+H]⁺: 324.11, found 324.2.

General procedure D in Scheme 1b (Reductive amination). To a solution of amine (0.06 mmol) in 0.5 mL MeOH was added to the aldehyde (0.08 mmol) followed by 2 drops of AcOH. The resulting mixture was stirred for 30 min before NaBH₃CN (0.1 mmol) was added.¹⁷ After stirring for 2 h at room temperature, the reaction was quenched with saturated NaHCO₃ and extracted with EA (3×). The combined organic layers were dried over Na₂SO₄, filtered, and concentrated. The residue was purified with silica gel column to provide the desired product.

3-(3-((((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)methyl)amino)prop-1-yn-1-yl) benzamide (4). To a mixture of 3 (570 mg, 1.76 mmol) in DCM (10 mL) was added DMP (1.49 g, 3.52 mmol) in one portion at 0° C. under N₂. The mixture was stirred at 0° C. for 3 hours. The reaction mixture was quenched with Na₂S₂O₃ (2× mass of alcohol) in saturated NaHCO₃ and stirred for 10 minutes. The reaction mixture was extracted with DCM, and the organic layer was washed twice with brine and dried with Na₂SO₄ to afford the crude aldehyde, which was used without any further purification.

To a solution of the crude aldehyde (100 mg, 0.3 mmol) and 3-(3-aminoprop-1-yn-1-yl)benzamide (65.0 mg, 0.37 mmol) in MeOH (2 mL) was added catalytic AcOH and stirred at room temperature for 30 minutess before addition of NaCNBH₃ (29.3 mg, 0.46 mmol) and the reaction was further stirred for 2 hours. After completion, the reaction mixture was diluted with MeOH, concentrated, and purified by flash column to afford 4 (108 mg, 59%). ¹H NMR (500 MHz, Methanol-d₄) δ 8.57 (s, 1H), 8.50 (s, 1H), 7.95 (t, J=1.8 Hz, 1H), 7.86-7.81 (m, 1H), 7.73 (d, J=3.7 Hz, 1H), 7.63-7.58 (m, 1H), 7.45 (t, J=7.8 Hz, 1H), 6.70 (d, J=3.7 Hz, 1H), 5.21-5.12 (m, 1H), 5.07-4.98 (m, 1H), 4.67-4.60 (m, 1H), 3.77 (s, 2H), 3.10-3.04 (m, 1H), 3.00-2.93 (m, 1H), 2.56-2.40 (m, 2H), 2.30-2.17 (m, 1H), 1.56 (s, 3H), 1.31 (s, 3H). ¹³C NMR (126 MHz, MeOD) δ 167.63, 151.38, 150.96, 149.77, 134.31, 134.05, 130.50, 128.81, 128.37, 127.07, 123.29, 117.80, 113.78, 99.30, 84.06, 83.17, 82.91, 61.36, 50.73, 43.34, 37.84, 35.48, 26.39, 23.99. ESI-MS (m/z) calculated for C₂₅H₂₇ClN₅O₃ [M+H]⁺: 480.17, found 480.1

General procedure E in Scheme 1b (Acetylation and deprotection). To a solution of secondary amine (1 mmol) in THF was added TEA (1.2 mmol) and acid chloride (1 mmol) (Iyamu et al., Angew Chemie Int Ed 202114813: 1-8 (2022)). The reaction was stirred until complete (1 hour). The protected inhibitor was stirred in a mixture of TFA: H2O (4:1) for 1 hour. The resulting solution was concentrated, dissolved in methanol, and purified by reverse HPLC to get the corresponding bisubstrate compound.

General procedure F in Scheme 1b (Reductive amination and deprotection). To a solution of amine (0.06 mmol) in 0.5 mL MeOH was added to the aldehyde (0.08 mmol) followed by 2 drops of AcOH. The resulting mixture was stirred for 30 minutes before NaBH₃CN (0.1 mmol) was added (Chen et al., J Med Chem 62(23): 10783-10797 (2019)). After stirring for 2 hours at room temperature, the reaction was quenched with water, extracted with EA, and concentrated. The crude was stirred in a mixture of TFA: H2O (4:1) for 1 hour. The resulting solution was concentrated, dissolved in methanol, and purified by reverse HPLC to get the corresponding bisubstrate compound.

3-(3-((((1R,2R,3S,4R)-4-(4-chloro-7H-pyrrolo[2,3-d]pyrimidin-7-yl)-2,3dihydroxycyclopentyl)methyl)(ethyl)amino)prop-1-yn-1-yl)benzamide (II542). To a solution of 4 (30 mg, 63 μmol) and iodoethane (9 mg, 0.06 mmol) in DMF (1 mL) was added K₂CO₃ (26 mg, 0.19 mmol) and heating at 40° C. until complete. The reaction mixture was diluted with water and extracted with EA. The organic layer was washed with brine, concentrated, and immediately treated with TFA: H2O (1:1) for 2 h and purified by prep HPLC to afford compound II542 (13 mg, 51%) as a clear oil. ¹H NMR (500 MHz, Methanol-d₄) δ 8.53 (s, 1H), 8.05 (s, 1H), 7.96-7.90 (m, 1H), 7.70 (d, J=7.9 Hz, 1H), 7.66 (d, J=3.7 Hz, 1H), 7.50 (t, J=7.8 Hz, 1H), 6.70 (d, J=3.7 Hz, 1H), 5.07-5.00 (m, 1H), 4.53 (s, 2H), 4.44 (t, J=6.1 Hz, 1H), 4.22 (t, J=6.4 Hz, 1H), 3.72-3.64 (m, 1H), 3.63-3.56 (m, 1H), 3.56-3.46 (m, 2H), 2.66-2.51 (m, 2H), 2.06-1.96 (m, 1H), 1.46 (t, J=7.3 Hz, 3H). ¹³C NMR (126 MHz, MeOD) δ 170.91, 152.82, 152.26, 151.01, 136.00, 135.70, 132.43, 130.87, 130.07, 129.73, 122.61, 119.43, 100.53, 90.49, 78.57, 76.14, 75.21, 63.23, 57.54, 50.76, 44.00, 40.53, 32.24, 9.63. HRMS (m/z) calculated for C₂₄H₂₇ClN₅O₃ [M+H]+: 468.1797, found 468.1799.

3-(3-(N-(((1R,2R,3S,4R)-4-(4-amino-7H-pyrrolo[2,3-d]pyrimidin-7-yl)-2,3-dihydroxycyclopentyl)methyl)acetamido)prop-1-yn-1-yl) benzamide (II642). Compound II642 was synthesized following an earlier reported procedure (Lambertucci et al., Bioorganic Med Chem 17: 2812-2822 (2009)). To a solution of (II399) (25 mg, 0.048 mmol) in dioxane 0.5 mL in a sealed tube was added ammonium hydroxide (0.5 mL), and the mixture was heated to 100° C. for 8 hours and purified by prep HPLC to afford II642 (20 mg) as a mixture of rotamers. ¹H NMR (500 MHz, Methanol-d₄) δ 8.22-8.10 (m, 1H), 7.96-7.88 (m, 1H), 7.88-7.79 (m, 1H), 7.63-7.49 (m, 2H), 7.48-7.37 (m, 1H), 6.92-6.81 (m, 1H), 5.17-5.02 (m, 1H), 4.60-4.40 (m, 3H), 4.07-3.88 (m, 1H), 3.73 (d, J=8.0 Hz, 1H), 3.17 (s, 1H), 2.62-2.53 (m, 1H), 2.52-2.38 (m, 1H), 2.26 (d, J=21.2 Hz, 3H), 1.90-1.73 (m, 1H). ¹³C NMR (126 MHz, MeOD) δ 174.11, 171.20, 152.63, 149.03, 142.31, 135.75, 135.46, 131.89, 129.87, 129.77, 128.85, 128.59, 127.70, 127.26, 124.43, 124.02, 103.35, 102.84, 86.05, 84.53, 76.97, 76.47, 74.41, 74.10, 62.79, 61.89, 50.14, 44.20, 43.16, 40.22, 36.61, 32.49, 32.23, 21.78. HRMS (m/z) calculated for C₂₄H₂₇N₆O₂ [M+H]+: 463.2088, found 463.2087.

3-(3-(N-(((2R,3S,4R,5R)-5-(6-amino-9H-purin-9-yl)-3,4-dihydroxytetrahydrofuran-2-yl)methyl)acetamido)prop-1-yn-1-yl)benzamide (II661). To a solution of 3-(3-((((2R,3S,4R,5R)-5-(6-amino-9H-purin-9-yl)-3,4-dihydroxytetrahydrofuran-2-yl)methyl)amino)prop-1-yn-1-yl)benzamide (10 mg, 0.024 mmol) in THF 0.2 mL were added pyridine (2.8 mg 0.035 mmol) and acetic anhydride (2.7 mg, 0.026 mmol), and the mixture was stirred for 1 hour and purified by prep HPLC to afford compound II661 (7.6 mg, 69%) as a mixture of rotamers. ¹H NMR (500 MHz, Methanol-d₄) δ 8.46 (d, J=36.0 Hz, 1H), 8.34 (d, J=9.6 Hz, 1H), 7.90 (t, J=1.8 Hz, 1H), 7.87-7.78 (m, 1H), 7.58-7.51 (m, 1H), 7.47-7.39 (m, 1H), 6.05 (dd, J=8.5, 4.4 Hz, 1H), 4.70 (dd, J=5.6, 3.5 Hz, 1H), 4.55-4.47 (m, 1H), 4.45 (s, 1H), 4.43-4.36 (m, 1H), 4.36-4.27 (m, 1H), 4.08-3.95 (m, 1H), 3.80 (dd, J=14.2, 8.4 Hz, 1H), 2.19 (d, J=69.3 Hz, 3H). ¹³C NMR (126 MHz, MeOD) δ 173.95, 173.52, 171.23, 153.40, 150.12, 149.91, 147.97, 147.64, 143.89, 143.75, 135.68, 135.44, 131.92, 131.87, 129.84, 129.77, 128.78, 128.56, 124.44, 124.04, 120.90, 91.73, 90.78, 86.46, 85.88, 84.46, 84.12, 83.56, 75.02, 74.93, 73.32, 72.69, 51.27, 41.10, 37.00, 30.79, 23.74, 21.69. HRMS (m/z) calculated for C₂₂H₂₄N₇O₅ [M+H]+: 466.1833, found 466.1834.

In parallel, the structure-activity relationship (SAR) of the benzamide was examined by removing the amino group, incorporating lipophilic groups on the aromatic ring, or substituting the benzamide with other aromatic groups. The oxidation and reductive amination of 3 with propargyl amine afforded compound 14. After acetylation of the secondary amine, Sonogoshira coupling followed by deprotection afforded the analogues 5l-5r (Scheme 2), in which 5m is II562 and 5n is II559.

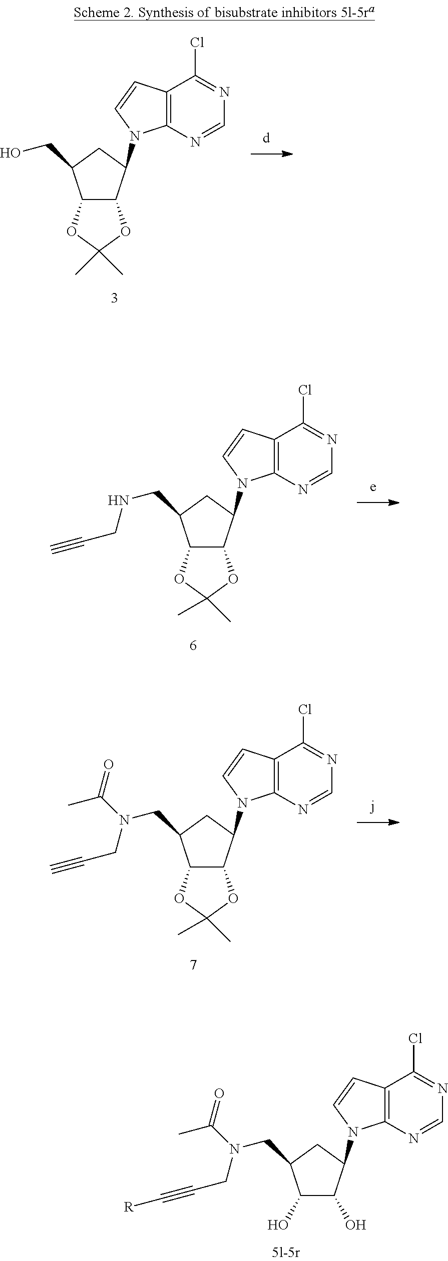

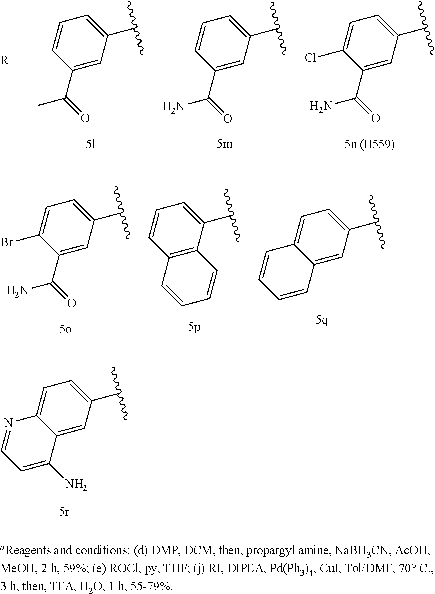

The substitution of the Cl group with a CF₃ group has been reported to improve metabolic stability (Tang et al., Carcinogenesis 32: 138-145 (2011)). An analogue containing CF₃ was synthesized according to Scheme 3. Alkylation of ethyl 4,4,4-trifluoro-3-oxobutanoate with bromo propene followed by cyclization with formimidamide afforded the pyrimidine 9, which was chlorinated with POCl₃. The oxidative cleavage of the alkene 10 afforded the aldehyde 11, which was protected (Scheme 3) and used for the synthesis of the trifluoromethyl analogues according to Scheme 1.

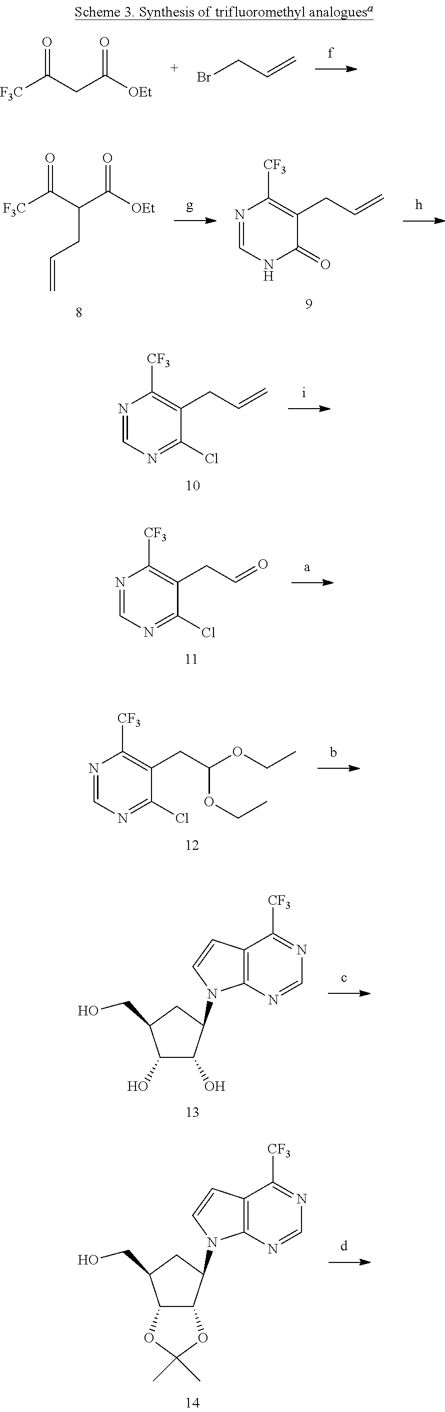

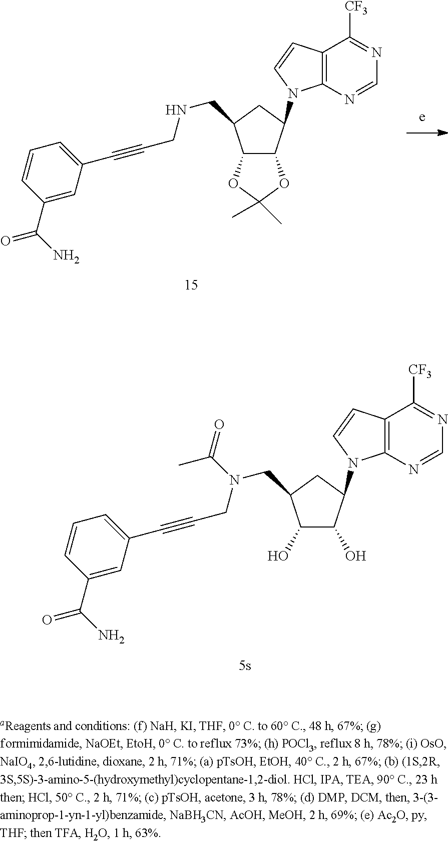 [SMILES: CC1(C)O[C@@H]2[C@@H](CNCC#Cc3cccc(C(N)=O)c3)C[C@@H](n3ccc4c(C(F)(F)F)ncnc43)[C@@H]2O1 ; CC(=O)N(CC#Cc1cccc(C(N)=O)c1)C[C@H]1C[C@@H](n2ccc3c(C(F)(F)F)ncnc32)[C@H](O)[C@@H]1O]

5-allyl-6-(trifluoromethyl)pyrimidin-4(3H)-one (8). To a solution of ethyl 2-(2,2,2-trifluoroacetyl)pent-4-enoate (6000 mg, 1 Eq, 1 mmol) in ethanol (1 mL) was added formimidamide.HCl (1.1 mmol) and sodium ethoxide (1.2 mmol) 21% in EA. The mixture was heated under reflux for 16 hours. After cooling to room temperature, the solvent was removed under reduced pressure. The residue was suspended with water (1mL), acidified with 1 M HCl, extracted with Et₂O (2×), and dried with Na₂SO₄. The solvent was removed under reduced pressure and purification by silica gel chromatography afforded compound 8. ¹H NMR (500 MHz, Chloroform-d) δ 8.25-8.19 (m, 1H), 5.98-5.81 (m, 1H), 5.20-5.05 (m, 2H), 3.54-3.41 (m, 2H). ¹³C NMR (126 MHz, CDCl₃) δ164.40, 149.69, 149.42, 149.14, 146.72, 132.88, 128.13, 122.47, 120.27, 117.53, 29.15.

5-allyl-4-chloro-6-(trifluoromethyl)pyrimidine (9). A suspension of 8 (1 mmol) in POCl3 (5.26 mL) was heated to reflux for 17 h. After cooling to room temperature, the mixture was carefully poured on 0° C. cold water (10 mL) under vigorous stirring. The aqueous phase was extracted with Et2O (2×) and dried with Na2SO4. The solvent was removed under reduced pressure and purification by silica gel afforded compound 9. ¹H NMR (500 MHz, Chloroform-d) δ 8.98 (s, 1H), 5.92-5.80 (m, 1H), 5.23-5.15 (m, 1H), 5.12-5.03 (m, 1H), 3.73-3.64 (m, 2H). ¹³C NMR (126 MHz, CDCl₃) δ 164.93, 156.23, 154.51, 131.60, 130.84, 122.00, 119.80, 118.66, 32.39.

2-(4-chloro-6-(trifluoromethyl)pyrimidin-5-yl)acetaldehyde (10). To a solution of 9 (1 mmol) in dioxane (4 mL) and water (2 mL) was added osmium tetroxide (0.01 mmol) and was stirred for 15 minutes during which time the solution became dark brown (due to osmate ester formation). 2,6-lutidine (2 mmol) was then added followed by portion-wise addition of sodium periodate (4 mmol) over 30 minutes. The tan-colored slurry was then stirred for additional 1.5 hours. The reaction was diluted with water and extracted with DCM (50 mL×3). The organic layer was washed with dilute HCl, brine and then dried with sodium sulfate. The DCM was removed under reduced pressure and the residue was purified by flash chromatography to afford compound 10. ¹H NMR (500 MHz, Chloroform-d) δ 9.93-9.70 (m, 1H), 9.19-8.97 (m, 1H), 4.17 (d, J=8.7 Hz, 2H). ¹³C NMR (126 MHz, CDCl₃) δ 165.32, 165.27, 136.87, 129.11, 129.07, 96.76, 93.52, 91.31, 15.05.

4-chloro-5-(2,2-diethoxyethyl)-6-(trifluoromethyl)pyrimidine (11). Compound 11 was synthesized according to general procedure B. ¹H NMR (500 MHz, Chloroform-d) δ 8.95 (s, 1H), 4.78-4.71 (m, 1H), 3.74-3.65 (m, 2H), 3.48-3.37 (m, 2H), 3.36-3.29 (m, 2H), 1.15-1.05 (m, 6H). ¹³C NMR (126 MHz, CDCl₃) δ 165.43, 156.07, 155.31, 155.03, 128.79, 122.03, 101.51, 63.15, 33.67, 15.18.

4-chloro-5-(2,2-diethoxyethyl)-6-(trifluoromethyl)pyrimidine (12). Compound 12 was synthesized according to general procedure A. ¹H NMR (500 MHz, Chloroform-d) δ 8.95 (s, 1H), 4.78-4.71 (m, 1H), 3.74-3.65 (m, 2H), 3.48-3.37 (m, 2H), 3.36-3.29 (m, 2H), 1.15-1.05 (m, 6H). ¹³C NMR (126 MHz, CDCl₃) δ 165.43, 156.07, 155.31, 155.03, 128.79, 122.03, 119.83, 101.51, 63.15, 33.67, 15.18. ¹³C NMR (126 MHz, CDCl₃) δ 165.43, 156.07, 155.16 (q, J=228.9), 128.79, 121.00 (d, J=277.5), 101.51, 63.15, 33.67, 15.18. ESI-MS (m/z) calculated for C₁₁H₁₅ClF₃N₂O₂ [M+H]⁺: 299.07, found 299.0

(1S,2R,3R,5R)-3-(hydroxymethyl)-5-(4-(trifluoromethyl)-7H-pyrrolo[2,3-d]pyrimidin-7-yl)cyclopentane-1,2-diol (13). To a mixture of 12 (300 mg, 0.91 mmol) and (1S,2R,3S,5S)-3-amino-5-(hydroxymethyl)cyclopentane-1,2-diol. HCl (185 mg, 1.01 mmol) in IPA: H₂O (7:1) 8 mL was added TEA (320 μL, 2.30 mmol) in one portion at room temperature under N₂. The reaction was heated to 90° C. and stirred for 23 hours. The mixture was cooled to 50° C. and 4 M HCl (0.68 mL, 2.75 mmol) was added slowly. The reaction was then stirred at 50° C. for 2 hours. The reaction was cooled to room temperature and NaHCO₃ was added slowly. The reaction mixture was extracted with EA, dried with Na₂SO₄, filtered and purified by flash column eluting with 8% MeOH in DCM to afford compound 13 as a yellow solid. ¹H NMR (500 MHz, Methanol-d₄) δ 8.88 (s, 1H), 7.89 (d, J=3.8 Hz, 1H), 6.83-6.65 (m, 1H), 5.30-5.19 (m, 1H), 4.49-4.42 (m, 1H), 4.12-4.04 (m, 1H), 3.72 (d, J=5.8 Hz, 2H), 2.48-2.37 (m, 1H), 2.33-2.23 (m, 1H), 1.92-1.80 (m, 1H). ¹³C NMR (126 MHz, MeOD) δ 154.27, 151.05, 146.42 (q, J=227.0), 146.10, 132.32, 123.15 (d, J=272.9), 116.39, 99.99, 77.04, 73.71, 64.47, 61.35, 46.65, 30.24. ESI-MS (m/z) calculated for C₁₃H₁₅F₃N₃O₃ [M+H]⁺: 311.10, found 311.5.

((3aR,4R,6R,6aS)-2,2-dimethyl-6-(4-(trifluoromethyl)-7H-pyrrolo[2,3-d]pyrimidin-7-yl)tetrahydro-4H-cyclopenta[d][1,3]dioxol-4-yl) methanol (14). Compound 14 was synthesized according to general procedure B. ¹H NMR (500 MHz, Methanol-d₄) δ 8.89 (s, 1H), 7.88 (d, J=3.8 Hz, 1H), 6.80-6.72 (m, 1H), 5.27-5.18 (m, 1H), 4.97 (t, J=6.7 Hz, 1H), 4.65 (dd, J=7.2, 4.4 Hz, 1H), 3.75-3.66 (m, 2H), 2.46-2.35 (m, 2H), 2.30-2.19 (m, 1H), 1.54 (s, 3H), 1.27 (s, 3H). ¹³C NMR (126 MHz, MeOD) δ 153.96, 151.30, 146.78 (q, J=225.0), 132.20, 122.98 (d, J=272.9), 116.28, 114.69, 100.27, 85.39, 82.55, 63.85, 62.61, 46.95, 34.92, 27.87, 25.42. ESI-MS (m/z) calculated for C₁₆H₁₉F₃N₃O₃ [M+H]⁺: 358.13, found 358.1.

3-(3-((((3aR,4R,6R,6aS)-2,2-dimethyl-6-(4-(trifluoromethyl)-7H-pyrrolo[2,3-d]pyrimidin-7-yl)tetrahydro-4H-cyclopenta[d][1,3]dioxol-4-yl)methyl)amino)prop-1-yn-1-yl)benzamide (15). To a mixture of 14 (170 mg, 0.47 mmol) in DCM (5 mL) was added DMP (404 mg, 0.95 mmol) in one portion at 0° C. under N₂. The mixture was stirred at 0° C. for 3 hours. The reaction mixture was quenched with Na₂S₂O₃ (2× mass of alcohol) in sat. NaHCO₃ and stirred for 10 minutes. The reaction mixture was extracted with DCM and the organic layer was washed twice with brine, dried with Na₂SO₄ to afford the crude aldehyde, which was used without any further purification.

To a solution of the crude aldehyde (100 mg, 0.28 mmol) and 3-(3-aminoprop-1-yn-1-yl)benzamide (58.0 mg, 0.33 mmol) in MeOH (2 mL) was added catalytic AcOH and stirred at room temperature for 30 minutes before addition of NaCNBH₃ (21.2 mg, 0.33 mmol) and the reaction was further stirred for 2 hours. After completion, the reaction mixture was diluted with MeOH, concentrated, and purified by flash column to afford 15. ¹H NMR (500 MHz, Methanol-d₄) δ 8.91 (s, 1H), 8.00 (s, 1H), 7.94-7.84 (m, 2H), 7.63 (t, J=10.7 Hz, 1H), 7.47 (t, J=7.7 Hz, 1H), 6.88-6.73 (m, 1H), 5.33-5.22 (m, 1H), 5.18-5.02 (m, 2H), 4.72 (t, J=6.2 Hz, 1H), 4.07 (s, 2H), 3.21 (d, J=9.9 Hz, 1H), 2.64-2.49 (m, 2H), 2.32 (d, J=12.5 Hz, 1H), 1.57 (s, 3H), 1.31 (s, 3H). ¹³C NMR (126 MHz, MeOD) δ 172.07, 153.92, 151.38, 146.94 (q, J=226.7), 135.84, 135.55, 132.58, 132.11, 129.89, 129.06, 123.69, 122.94, 116.44, 115.58, 100.40, 86.86, 85.40, 83.85, 83.63, 62.18, 50.92, 43.71, 38.77, 36.57, 27.75, 25.39. ESI-MS (m/z) calculated for C₂₆H₂₇F₃N₅O₃ [M+H]⁺: 514.20, found 514.1.

To investigate the effect of the chloro group on the adenine portion of the molecule, the dechlorinated analogue 5t and N⁶ amino analogue 5u were synthesized according to Scheme 4.

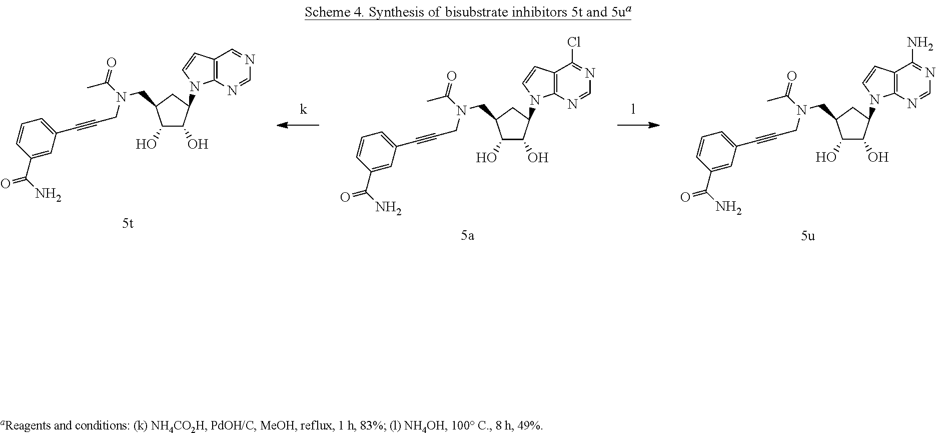 [SMILES: CC(=O)N(CC#Cc1cccc(C(N)=O)c1)C[C@H]1C[C@@H](n2ccc3cncnc32)[C@H](O)[C@@H]1O ; CC(=O)N(CC#Cc1cccc(C(N)=O)c1)C[C@H]1C[C@@H](n2ccc3c(Cl)ncnc32)[C@H](O)[C@@H]1O ; CC(=O)N(CC#Cc1cccc(C(N)=O)c1)C[C@H]1C[C@@H](n2ccc3c(N)ncnc32)[C@H](O)[C@@H]1O]

3-(3-(N-(((1R,2R,3S,4R)-4-(4-chloro-7H-pyrrolo[2,3-d]pyrimidin-7-yl)-2,3-dihydroxycyclopentyl)methyl)acetamido)prop-1-yn-1-yl)benzamide (II399). A solution of 4 (60 mg, 0.13 mmol) and pyridine (20 mg, 0.25 mmol) in THF (2 mL) was added acetic anhydride (26 mg, 0.25 mmol). The reaction was stirred at room temperature for 1 h, concentrated and immediately treated with TFA: H2O (1:1) for 2 h and purified by prep HPLC to afford compound II399 (40 mg, 69%) as a mixture of rotamers. ¹H NMR (500 MHz, Methanol-d₄) δ 8.52 (d, J=2.7 Hz, 1H), 7.97-7.91 (m, 1H), 7.88-7.80 (m, 1H), 7.73-7.65 (m, 1H), 7.62-7.54 (m, 1H), 7.46-7.38 (m, 1H), 6.66 (t, J=3.7 Hz, 1H), 5.14-4.97 (m, 1H), 4.61-4.41 (m, 3H), 4.09-3.88 (m, 1H), 3.82-3.75 (m, 1H), 3.74-3.65 (m, 1H), 2.64-2.39 (m, 2H), 2.26 (d, J=12.4 Hz, 3H), 1.99-1.79 (m, 1H). ¹³C NMR (126 MHz, MeOD) δ 174.06, 152.65, 152.53, 150.95, 135.75, 135.47, 131.90, 130.95, 130.28, 129.85, 129.75, 128.86, 128.60, 124.03, 119.23, 100.41, 100.28, 86.01, 84.53, 76.65, 76.14, 74.50, 74.30, 63.13, 62.05, 52.54, 50.16, 44.06, 43.17, 40.26, 36.46, 31.90, 21.77. HRMS (m/z) calculated for C₂₄H₂₅ClN₅O₄ [M+H]⁺: 482.1589, found 482.1588.

3-(3-(N-(((1R,2R,3S,4R)-4-(4-chloro-7H-pyrrolo[2,3-d]pyrimidin-7-yl)-2,3-dihydroxycyclopentyl)methyl)propionamido)prop-1-yn-1-yl)benzamide (5b). Compound 5b was prepared accroding to general procedure E as a mixture of rotamers. ¹H NMR (500 MHz, Methanol-d₄) δ 8.57-8.43 (m, 1H), 7.96-7.90 (m, 1H), 7.88-7.76 (m, 1H), 7.74-7.63 (m, 1H), 7.61-7.52 (m, 1H), 7.47-7.33 (m, 1H), 6.71-6.59 (m, 1H), 5.19-4.96 (m, 1H), 4.69-4.57 (m, 1H), 4.54-4.41 (m, 2H), 4.08-3.96 (m, 1H), 3.94-3.75 (m, 1H), 3.75-3.50) (m, 1H), 2.64-2.56 (m, 2H), 2.55-2.38 (m, 1H), 2.28-2.13 (m, 1H), 2.00-1.77 (m, 1H), 1.20-1.12 (m, 3H). ¹³C NMR (126 MHz, MeOD) δ 177.04, 152.65, 152.54, 152.41, 150.94, 135.75, 135.65, 135.48, 131.88, 130.99, 130.32, 129.85, 129.75, 128.83, 128.59, 124.06, 119.24, 100.41, 100.28, 100.15, 86.26, 84.38, 76.65, 76.11, 74.50, 74.29, 74.00, 63.12, 62.07, 51.63, 50.29, 44.10, 43.16, 40.00, 39.44, 36.65, 32.37, 31.98, 31.90, 27.58, 9.81. HRMS (m/z) calculated for C₂₅H₂₇ClN₅O₄ [M+H]⁺: 496.1746, found 496.1744.

3-(3-(N-(((1R,2R,3S,4R)-4-(4-chloro-7H-pyrrolo[2,3-d]pyrimidin-7-yl)-2,3-dihydroxycyclopentyl)methyl)pivalamido)prop-1-yn-1-yl)benzamide (5c). Compound 5c was prepared according to general procedure E as a mixture of rotamers. ¹H NMR (500 MHz, Methanol-d₄) δ 8.53 (s, 1H), 7.94 (t, J=1.7 Hz, 1H), 7.88-7.82 (m, 1H), 7.70 (d, J=3.7 Hz, 1H), 7.60-7.56 (m, 1H), 7.43 (t, J=7.8 Hz, 1H), 6.67 (d, J=3.6 Hz, 1H), 5.09-5.01 (m, 1H), 4.72-4.59 (m, 2H), 4.57-4.48 (m, 1H), 4.07-4.00 (m, 1H), 3.95-3.85 (m, 1H), 3.76-3.66 (m, 1H), 2.67-2.55 (m, 1H), 2.49-2.36 (m, 1H), 1.94-1.81 (m, 1H), 1.37 (s, 9H). ¹³C NMR (126 MHz, MeOD) δ 180.07, 152.57, 150.91, 135.72, 135.46, 131.86, 130.56, 129.83, 128.78, 124.19, 119.31, 100.34, 86.76, 84.48, 76.54, 74.60, 62.46, 51.31, 43.01, 40.25, 31.96, 28.94. HRMS (m/z) calculated for C₂₇H₃₁ClN₅O₄ [M+H]⁺: 524.2059, found 524.2058.

3-(3-(N-(((1R,2R,3S,4R)-4-(4-chloro-7H-pyrrolo[2,3-d]pyrimidin-7-yl)-2,3-dihydroxycyclopentyl)methyl)cyclohexanecarboxamido)prop-1-yn-1-yl)benzamide (5d). Compound 5d was prepared according to general procedure E as a mixture of rotamers. ¹H NMR (500 MHz, Methanol-d₄) δ 8.53 (d, J=1.3 Hz, 1H), 7.97-7.90 (m, 1H), 7.88-7.80 (m, 1H), 7.69 (dd, J=21.4, 3.7 Hz, 1H), 7.60-7.53 (m, 1H), 7.47-7.37 (m, 1H), 6.67 (t, J=3.3 Hz, 1H), 5.22-4.97 (m, 1H), 4.66-4.55 (m, 3H), 4.54-4.40 (m, 2H), 4.11-3.93 (m, 1H), 3.86-3.65 (m, 2H), 2.92-2.73 (m, 1H), 2.61-2.35 (m, 2H), 1.91-1.77 (m, 4H), 1.76-1.69 (m, 1H), 1.56-1.48 (m, 1H), 1.47-1.36 (m, 2H), 1.34-1.19 (m, 2H). ¹³C NMR (126 MHz, MeOD) δ 179.51, 177.87, 152.63, 152.55, 150.94, 135.68, 131.86, 131.14, 130.35, 129.88, 128.81, 128.57, 124.08, 119.25, 100.42, 86.71, 84.46, 76.69, 76.12, 74.49, 62.09, 52.50, 50.19, 45.89, 43.14, 42.46, 39.52, 36.43, 31.84, 30.74, 30.62, 26.95, 26.74. HRMS (m/z) calculated for C₂₉H₃₃ClN₅O₄ [M+H]⁺: 550.2216, found 550.2214.

3-(3-(N-(((1R,2R,3S,4R)-4-(4-chloro-7H-pyrrolo[2,3-d]pyrimidin-7-yl)-2,3-dihydroxycyclopentyl)methyl)-2-cyclohexylacetamido) prop-1-yn-1-yl)benzamide (5e). Compound 5e was prepared according to general procedure E as a mixture of rotamers. ¹H NMR (500 MHz, Methanol-d₄) δ 8.54-8.44 (m, 1H), 7.96-7.90 (m, 1H), 7.89-7.79 (m, 1H), 7.74-7.63 (m, 1H), 7.60-7.52 (m, 1H), 7.42 (dt, J=14.9, 7.6 Hz, 1H), 6.69-6.60 (m, 1H), 5.20-4.96 (m, 1H), 4.69-4.43 (m, 4H), 4.10-3.98 (m, 1H), 3.86-3.76 (m, 1H), 3.75-3.67 (m, 1H), 2.57 (tt, J=8.9, 4.5 Hz, 1H), 2.46-2.39 (m, 2H), 1.99-1.83 (m, 2H), 1.82-1.74 (m, 2H), 1.73-1.59 (m, 3H), 1.34-1.24 (m, 3H), 1.23-1.13 (m, 1H), 1.08-0.97 (m, 2H). ¹³C NMR (126 MHz, MeOD) δ 177.48, 175.61, 174.88, 172.28, 152.65, 152.54, 152.39, 150.95, 150.86, 135.69, 135.52, 131.90, 131.15, 130.33, 129.85, 129.74, 128.80, 128.57, 124.08, 119.44, 119.25, 100.41, 100.26, 100.12, 86.83, 86.52, 84.47, 83.43, 79.31, 76.68, 76.07, 74.58, 74.23, 74.04, 63.27, 62.54, 62.05, 51.90, 50.18, 44.21, 43.08, 41.92, 41.67, 40.02, 39.79, 36.75, 36.68, 36.33, 34.40, 34.30, 31.95, 27.32, 27.28. HRMS (m/z) calculated for C₃₀H₃₅ClN₅O₄ [M+H]⁺: 564.2372, found 564.2370.

N-(3-(3-carbamoylphenyl)prop-2-yn-1-yl)-N-(((1R,2R,3S,4R)-4-(4-chloro-7H-pyrrolo[2,3-d]pyrimidin-7-yl)-2,3-dihydroxycyclopentyl)methyl)benzamide (5f). Compound 5f was prepared according to general procedure E as a mixture of rotamers. ¹H NMR (500 MHz, Methanol-d₄) δ 8.60-8.33 (m, 1H), 8.04-7.92 (m, 1H), 7.92-7.84 (m, 1H), 7.81-7.67 (m, 1H), 7.63-7.54 (m, 2H), 7.52-7.42 (m, 5H), 6.75-6.57 (m, 1H), 5.19-4.94 (m, 1H), 4.66-4.53 (m, 2H), 4.48-4.34 (m, 1H), 4.28-4.12 (m, 1H), 4.08-3.83 (m, 2H), 2.83-2.41 (m, 1H), 2.09-1.91 (m, 1H). ¹³C NMR (126 MHz, MeOD) δ 174.53, 171.26, 152.57, 150.92, 137.02, 135.76, 135.46, 131.92, 131.36, 130.48, 129.85, 128.88, 127.77, 122.00, 119.33, 100.40, 86.11, 76.63, 74.69, 62.37, 42.99, 41.64, 31.98. HRMS (m/z) calculated for C₂₉H₂₇ClN₅O₄ [M+H]⁺: 544.1746, found 544.1747.

3-(3-(N-(((1R,2R,3S,4R)-4-(4-chloro-7H-pyrrolo[2,3-d]pyrimidin-7-yl)-2,3-dihydroxycyclopentyl)methyl)-2-hydroxyacetamido)prop-1-yn-1-yl)benzamide (5g). Compound 5g was prepared according to general procedure E as a mixture of rotamers. ¹H NMR (500 MHz, Methanol-d₄) δ 8.61-8.40 (m, 1H), 8.03-7.80 (m, 2H), 7.77-7.51 (m, 2H), 7.51-7.35 (m, 1H), 6.73-6.60 (m, 1H), 5.25-5.01 (m, 2H), 4.70-4.59 (m, 1H), 4.57-4.23 (m, 4H), 4.17-3.97 (m, 1H), 3.86-3.40 (m, 2H), 3.22-2.92 (m, 1H), 2.68-2.39 (m, 1H), 2.31-2.14 (m, 1H), 2.06-1.78 (m, 1H). ¹³C NMR (126 MHz, MeOD) δ 176.75, 167.36, 152.53, 150.93, 135.76, 131.92, 131.07, 130.35, 129.86, 128.91, 123.93, 119.42, 100.41, 100.16, 83.80, 79.35, 76.62, 73.02, 62.44, 62.10, 56.95, 43.65, 43.07, 38.09, 32.60, 31.89. HRMS (m/z) calculated for C₂₄H₂₅ClN₅O₅ [M+H]⁺: 498.1539, found 498.1538.

N⁴-(3-(3-carbamoylphenyl)prop-2-yn-1-yl)-N⁴-(((1R,2R,3S,4R)-4-(4-chloro-7H-pyrrolo[2,3-d]pyrimidin-7-yl)-2,3-dihydroxycyclopentyl)methyl)-L-asparagine (5h). Compound 5h was prepared according to general procedure E as a mixture of rotamers. ¹H NMR (500 MHz, DMSO-d₆) δ 8.60 (d, J=3.0 Hz, 1H), 8.28 (d, J=56.7 Hz, 1H), 8.13-8.05 (m, 1H), 7.97-7.92 (m, 1H), 7.90-7.82 (m, 1H), 7.60-7.50 (m, 1H), 7.48-7.39 (m, 2H), 6.67 (d, J=3.6 Hz, 1H), 5.04-4.93 (m, 1H), 4.54 (t, J=17.7 Hz, 1H), 4.47-4.39 (m, 1H), 4.36-4.26 (m, 1H), 3.91-3.83 (m, 1H), 3.82-3.72 (m, 1H), 3.70-3.60 (m, 1H), 3.58-3.52 (m, 2H), 3.17-3.04 (m, 1H), 2.94-2.79 (m, 1H), 2.47-2.35 (m, 1H), 2.34-2.20 (m, 1H), 1.78-1.58 (m, 1H). ¹³C NMR (126 MHz, DMSO) δ 171.34, 170.55, 169.38, 166.97, 166.88, 150.93, 150.83, 150.53, 150.01, 134.61, 133.90, 133.82, 130.75, 130.43, 129.75, 129.47, 128.69, 127.95, 127.68, 122.15, 121.87, 117.04, 98.73, 86.47, 86.11, 83.22, 82.14, 74.96, 74.70, 72.35, 72.22, 60.30, 60.04, 50.98, 49.69, 48.90, 42.14, 41.73, 37.99, 34.73, 34.27, 31.07, 30.82. HRMS (m/z) calculated for C₂₆H₂₈ClN₆O₆ [M+H]⁺: 555.1753, found 555.1753.

3-(3-(N-(((1R,2R,3S,4R)-4-(4-chloro-7H-pyrrolo[2,3-d]pyrimidin-7-yl)-2,3-dihydroxycyclopentyl)methyl)-2-(5-oxopyrrolidin-2-yl)acetamido)prop-1-yn-1-yl) benzamide (5i). Compound 5i was prepared according to general procedure E as a mixture of rotamers. ¹H NMR (500 MHz, Methanol-d₄) δ 8.52 (d, J=5.1 Hz, 1H), 7.96-7.91 (m, 1H), 7.88-7.80 (m, 1H), 7.74-7.70 (m, 1H), 7.60-7.54 (m, 1H), 7.47-7.38 (m, 1H), 6.69-6.64 (m, 1H), 5.13-4.97 (m, 1H), 4.60-4.42 (m, 3H), 4.18-4.09 (m, 1H), 4.09-4.00 (m, 1H), 3.95-3.78 (m, 1H), 3.76-3.62 (m, 1H), 2.90-2.77 (m, 2H), 2.63-2.40 (m, 2H), 2.40-2.28 (m, 3H), 1.96-1.77 (m, 2H). ¹³C NMR (126 MHz, MeOD) δ 180.70, 173.41, 172.64, 171.18, 152.60, 152.53, 150.92, 135.75, 135.47, 131.91, 130.39, 130.33, 129.86, 129.76, 128.88, 128.61, 124.40, 123.95, 119.23, 100.44, 100.34, 86.61, 86.09, 84.61, 83.66, 76.67, 76.58, 76.11, 74.59, 74.48, 74.37, 74.30, 63.12, 62.17, 62.13, 53.06, 52.99, 51.52, 50.34, 50.22, 44.13, 43.16, 40.91, 39.55, 39.44, 36.62, 31.96, 30.80, 27.73. HRMS (m/z) calculated for C₂₈H₃₀ClN₆O₅ [M+H]⁺: 565.1961, found 565.1959.

3-(3-(N-(((1R,2R,3S,4R)-4-(4-chloro-7H-pyrrolo[2,3-d]pyrimidin-7-yl)-2,3-dihydroxycyclopentyl)methyl)-2-(5-oxopyrrolidin-3-yl)acetamido)prop-1-yn-1-yl)benzamide (5j). Compound 5j was prepared according to general procedure E as a mixture of rotamers. ¹H NMR (500 MHz, Methanol-d₄) δ 8.53 (d, J=2.8 Hz, 1H), 7.93 (dt, J=8.5, 1.8 Hz, 1H), 7.87-7.79 (m, 1H), 7.74-7.65 (m, 1H), 7.61-7.53 (m, 1H), 7.47-7.37 (m, 1H), 6.70-6.62 (m, 1H), 5.13-4.97 (m, 1H), 4.61-4.40 (m, 3H), 4.09-3.88 (m, 1H), 3.82-3.67 (m, 2H), 3.66-3.56 (m, 1H), 3.17-3.06 (m, 1H), 3.02-2.89 (m, 1H), 2.84-2.69 (m, 2H), 2.63-2.37 (m, 3H), 2.16-2.05 (m, 1H), 1.98-1.77 (m, 1H). ¹³C NMR (126 MHz, MeOD) δ 180.28, 174.14, 173.37, 171.18, 152.63, 152.54, 152.37, 150.95, 135.78, 135.47, 131.88, 130.90, 130.30, 129.88, 129.76, 128.92, 128.60, 124.42, 123.94, 119.42, 119.23, 100.44, 100.33, 86.65, 86.19, 84.57, 83.57, 76.66, 76.15, 74.51, 74.32, 63.09, 62.05, 51.55, 50.28, 44.13, 43.15, 39.46, 38.78, 37.65, 36.66, 32.63, 32.46, 32.09, 31.93. HRMS (m/z) calculated for C₂₈H₃₀ClN₆O₅ [M+H]⁺: 565.1961, found 565.1961.

3-(3-((((1R,2R,3S,4R)-4-(4-chloro-7H-pyrrolo[2,3-d]pyrimidin-7-yl)-2,3-dihydroxycyclopentyl)methyl)(propyl)amino)prop-1-yn-1-yl)benzamide (5k). Compound 5k was prepared according to general procedure E. ¹H NMR (500 MHz, Methanol-d₄) δ 8.52 (s, 1H), 8.04 (s, 1H), 7.92 (d, J=7.8 Hz, 1H), 7.73-7.63 (m, 2H), 7.54-7.46 (m, 1H), 6.68 (t, J=3.0 Hz, 1H), 5.18-5.03 (m, 1H), 4.72-4.61 (m, 1H), 4.50 (d, J=23.2 Hz, 2H), 4.43 (t, J=6.1 Hz, 1H), 4.29-4.19 (m, 1H), 3.72-3.56 (m, 1H), 3.43-3.35 (m, 2H), 2.66-2.51 (m, 1H), 2.44-2.23 (m, 1H), 2.06-1.95 (m, 1H), 1.88 (h, J=7.1 Hz, 2H), 1.08 (t, J=7.3 Hz, 3H). ¹³C NMR (126 MHz, MeOD) δ 152.78, 152.23, 150.97, 135.99, 132.40, 130.83, 130.04, 129.71, 122.58, 119.39, 100.49, 90.55, 79.06. 78.57, 76.12, 75.22, 63.20, 61.81, 58.00, 57.04, 45.03, 44.62, 40.47, 32.24, 18.68, 11.14. HRMS (m/z) calculated for C₂₅H₂₉ClN₅O₃ [M+H]⁺: 482.1953, found 482.1951.

3-(3-(butyl(((1R,2R,3S,4R)-4-(4-chloro-7H-pyrrolo[2,3-d]pyrimidin-7-yl)-2,3-dihydroxycyclopentyl)methyl)amino)prop-1-yn-1-yl)benzamide (5l). Compound 5l was prepared according to general procedure E. ¹H NMR (500 MHz, Methanol-d₄) δ 8.52 (d, J=3.0 Hz, 1H), 8.04 (d, J=2.1 Hz, 1H), 7.92 (d, J=7.8 Hz, 1H), 7.73-7.64 (m, 2H), 7.53-7.46 (m, 1H), 6.68 (t, J=3.2 Hz, 1H), 5.17-5.00 (m, 1H), 4.73-4.62 (m, 1H), 4.56-4.41 (m, 3H), 4.30-4.21 (m, 1H), 3.73-3.58 (m, 1H), 3.47-3.38 (m, 2H), 2.66-2.52 (m, 1H), 2.45-2.22 (m, 1H), 2.05-1.95 (m, 1H), 1.89-1.78 (m, 2H), 1.49 (h, J=7.4 Hz, 2H), 1.04 (t, J=7.4 Hz, 3H). ¹³C NMR (126 MHz, MeOD) δ 152.79, 152.24, 150.99, 135.99, 132.41, 130.84, 130.06, 129.72, 122.59, 119.40, 100.50, 100.29, 90.56, 78.60, 76.14, 75.21, 63.18, 61.84, 58.04, 55.36, 44.58, 40.50, 32.24, 27.11, 20.88, 13.90. HRMS (m/z) calculated for C₂₆H₃₁ClN₅O₃ [M+H]⁺: 496.2100, found 496.2101.

3-(3-((((1R,2R,3S,4R)-4-(4-chloro-7H-pyrrolo[2,3-d]pyrimidin-7-yl)-2,3-dihydroxycyclopentyl)methyl)(prop-2-yn-1-yl)amino)prop-1-yn-1-yl)benzamide (5m). Compound 5m was prepared according to general procedure E. ¹H NMR (500 MHz, Methanol-d₄) δ 8.53 (s, 1H), 8.07-8.02 (m, 1H), 7.95-7.89 (m, 1H), 7.73-7.64 (m, 2H), 7.50) (td, J=7.8, 5.9 Hz, 1H), 6.68 (d, J=3.6 Hz, 1H), 5.20-5.01 (m, 1H), 4.73-4.59 (m, 1H), 4.51 (s, 1H), 4.49-4.43 (m, 2H), 4.35-4.33 (m, 1H), 4.30 (d, J=2.6 Hz, 1H), 4.28-4.19 (m, 1H), 3.82-3.61 (m, 1H), 3.58-3.42 (m, 1H), 2.64-2.50 (m, 1H), 2.43-2.27 (m, 1H), 2.04-1.95 (m, 1H). HRMS (m/z) calculated for C₂₅H₂₅ClN₅O₃ [M+H]⁺: 498.1640, found 498.1639.

N-(3-(3-acetylphenyl)prop-2-yn-1-yl)-N-(((1R,2R,3S,4R)-4-(4-chloro-7H-pyrrolo[2,3-d]pyrimidin-7-yl)-2,3-dihydroxycyclopentyl)methylacetamide (5n). Compound 5n was prepared according to general procedure C as a mixture of rotamers. ¹H NMR (500 MHz, Methanol-d₄) δ 8.55-8.49 (m, 1H), 8.04-7.92 (m, 2H), 7.74-7.60 (m, 2H), 7.50)-7.43 (m, 1H), 6.69-6.64 (m, 1H), 5.13-4.98 (m, 1H), 4.60-4.43 (m, 3H), 4.11-3.88 (m, 1H), 3.84-3.63 (m, 2H), 2.58 (d, J=5.9 Hz, 3H), 2.54-2.38 (m, 1H), 2.27 (d, J=11.1 Hz, 3H), 2.01-1.79 (m, 1H). ¹³C NMR (126 MHz, MeOD) δ 199.42, 174.07, 152.65, 152.53, 152.37, 150.93, 138.60, 137.06, 132.48, 132.43, 130.94, 130.29, 130.07, 129.97, 129.57, 129.30, 124.65, 124.24, 119.42, 119.23, 100.41, 100.29, 86.81, 86.23, 84.39, 83.43, 76.64, 76.14, 74.49, 74.30, 63.14, 62.64, 62.07, 52.65, 50.24, 44.12, 43.18, 40.31, 36.58, 32.02, 31.94, 26.74, 21.78, 21.74. HRMS (m/z) calculated for C₂₅H₂₆ClN₄O₄ [M+H]⁺: 481.1637, found 481.1637.

N-(((1R,2R,3S,4R)-4-(4-chloro-7H-pyrrolo[2,3-d]pyrimidin-7-yl)-2,3-dihydroxycyclopentyl)methyl)-N-(3-(naphthalen-1-yl)prop-2-yn-1-yl)acetamide (5o). Compound 5o was prepared according to general procedure C as a mixture of rotamers. ¹H NMR (500 MHz, Methanol-d₄) δ 8.46 (d, J=15.6 Hz, 1H), 8.27-8.19 (m, 1H), 7.92-7.84 (m, 2H), 7.70-7.65 (m, 1H), 7.64-7.59 (m, 1H), 7.56-7.47 (m, 2H), 7.46-7.38 (m, 1H), 6.66-6.59 (m, 1H), 5.12-4.95 (m, 1H), 4.73-4.57 (m, 3H), 4.55-4.47 (m, 1H), 4.06-3.96 (m, 1H), 3.88-3.73 (m, 2H), 2.71-2.59 (m, 1H), 2.58-2.42 (m, 1H), 2.32 (d, J=30.4 Hz, 3H), 2.04-1.84 (m, 1H). HRMS (m/z) calculated for C₂₇H₂₆ClN₄O₃ [M+H]⁺: 489.1688, found 489.1686.

N-(((1R,2R,3S,4R)-4-(4-chloro-7H-pyrrolo[2,3-d]pyrimidin-7-yl)-2,3-dihydroxycyclopentyl)methyl)-N-(3-(naphthalen-2-yl)prop-2-yn-1-yl)acetamide (5p). Compound 5p was prepared according to general procedure C as a mixture of rotamers. ¹H NMR (500 MHz, Methanol-d₄) δ 8.49 (d, J=5.8 Hz, 1H), 7.97-7.90 (m, 1H), 7.87-7.74 (m, 3H), 7.73-7.65 (m, 1H), 7.53-7.47 (m, 2H), 7.46-7.39 (m, 1H), 6.67-6.62 (m, 1H), 5.14-4.98 (m, 1H), 4.64-4.54 (m, 2H), 4.54-4.44 (m, 2H), 4.10-3.92 (m, 1H), 3.82-3.71 (m, 1H), 2.66-2.43 (m, 2H), 2.28 (d, J=18.0 Hz, 3H), 2.01-1.81 (m, 1H). ¹³C NMR (126 MHz, MeOD) δ 174.11, 152.65, 152.53, 150.93, 134.37, 132.67, 132.56, 130.88, 130.24, 129.23, 129.15, 128.78, 128.05, 127.91, 127.80, 120.85, 119.21, 118.14, 100.43, 100.30, 85.75, 85.35, 76.66, 76.16, 74.51, 74.31, 63.10, 62.05, 49.85, 44.20, 43.25, 40.47, 36.68, 32.00, 21.81. HRMS (m/z) calculated for C₂₇H₂₆ClN₄O₃ [M+H]⁺: 489.1688, found 489.1687.

N-(3-(4-aminoquinolin-6-yl)prop-2-yn-1-yl)-N-(((1R,2R,3S,4R)-4-(4-chloro-7H-pyrrolo[2,3-d]pyrimidin-7-yl)-2,3-dihydroxycyclopentyl)methyl)acetamide (5q). Compound 5q was prepared according to general procedure C as a mixture of rotamers. ¹H NMR (500 MHz, Methanol-d₄) δ 8.46 (d, J=9.3 Hz, 1H), 8.38 (dd, J=27.7, 1.7 Hz, 1H), 8.31-8.23 (m, 1H), 7.90-7.82 (m, 1H), 7.77 (dd, J=14.1, 8.8 Hz, 1H), 7.69 (dd, J=20.1, 3.7 Hz, 1H), 6.78 (dd, J=6.7, 2.7 Hz, 1H), 6.64 (t, J=3.3 Hz, 1H), 5.13-5.00 (m, 1H), 4.62-4.46 (m, 3H), 4.10-4.00 (m, 1H), 3.98-3.77 (m, 1H), 3.76-3.67 (m, 1H), 2.67-2.57 (m, 1H), 2.56-2.40 (m, 1H), 2.28 (d, J=16.4 Hz, 3H), 2.02-1.81 (m, 1H). ¹³C NMR (126 MHz, MeOD) δ 174.05, 159.04, 152.59, 152.32, 150.87, 144.38, 136.98, 136.78, 130.88, 130.29, 127.94, 127.83, 122.79, 122.45, 122.24, 121.70, 119.37, 119.20, 117.86, 103.81, 100.40, 100.29, 88.34, 87.54, 83.98, 82.88, 76.58, 76.12, 74.53, 74.30, 63.08, 62.08, 52.96, 50.46, 44.17, 43.17, 40.42, 36.83, 32.12, 32.01, 21.82, 21.74. HRMS (m/z) calculated for C₂₆H₂₆ClN₆O₃ [M+H]⁺: 505.1749, found 505.1746.

2-chloro-5-(3-(N-(((1R,2R,3S,4R)-4-(4-chloro-7H-pyrrolo[2,3-d]pyrimidin-7-yl)-2,3-dihydroxycyclopentyl)methyl)acetamido)prop-1-yn-1-yl)benzamide (5r). Compound 5r was prepared according to general procedure C as a mixture of rotamers. ¹H NMR (500 MHz, Methanol-d₄) δ 8.52 (d, J=22.1 Hz, 1H), 7.75-7.65 (m, 1H), 7.63-7.55 (m, 1H), 7.54-7.46 (m, 1H), 7.40-7.31 (m, 1H), 6.71-6.62 (m, 1H), 5.18-4.97 (m, 1H), 4.68-4.53 (m, 1H), 4.53-4.49 (m, 1H), 4.49-4.39 (m, 1H), 4.08-3.97 (m, 1H), 3.93-3.72 (m, 1H), 3.72-3.50 (m, 1H), 2.62-2.52 (m, 1H), 2.51-2.38 (m, 1H), 2.25 (t, J=7.3 Hz, 3H), 1.98-1.77 (m, 1H). ¹³C NMR (126 MHz, MeOD) δ 174.03, 173.16, 172.15, 152.63, 152.54, 152.35, 150.95, 140.11, 140.03, 134.87, 134.65, 134.54, 132.56, 130.93, 130.26, 123.59, 123.15, 120.56, 120.23, 119.42, 119.21, 100.44, 100.32, 87.88, 87.27, 83.55, 82.55, 79.80, 79.32, 76.64, 76.13, 74.46, 74.26, 73.96, 63.06, 62.43, 61.98, 52.64, 50.15, 44.00, 43.12, 40.24, 39.87, 36.53, 32.33, 31.94, 31.87, 21.76, 21.73. HRMS (m/z) calculated for C₂₄H₂₄Cl₂N₅O₄ [M+H]⁺: 516.1200, found 516.1200.

5-(3-(N-(((1R,2R,3S,4R)-4-(4-chloro-7H-pyrrolo[2,3-d]pyrimidin-7-yl)-2,3-dihydroxycyclopentyl)methyl)acetamido)prop-1-yn-1-yl)-2-methylbenzamide (5s). Compound 5s was prepared according to general procedure C as a mixture of rotamers. ¹H NMR (500 MHz, Methanol-d₄) δ 8.56-8.47 (m, 1H), 7.73-7.63 (m, 1H), 7.50-7.43 (m, 1H), 7.40-7.32 (m, 1H), 7.25-7.15 (m, 1H), 6.69-6.63 (m, 1H), 5.16-4.97 (m, 1H), 4.70-4.57 (m, 1H), 4.57-4.48 (m, 2H), 4.47-4.38 (m, 1H), 4.16-3.98 (m, 1H), 3.96-3.73 (m, 1H), 3.73-3.48 (m, 1H), 2.62-2.47 (m, 1H), 2.43 (d, J=4.1 Hz, 3H), 2.31-2.21 (m, 3H), 2.02-1.74 (m, 1H). ¹³C NMR (126 MHz, MeOD) δ 174.42, 174.07, 172.58, 152.66, 152.55, 150.96, 137.89, 137.79, 133.89, 132.16, 132.07, 131.26, 130.95, 130.28, 121.49, 121.07, 119.44, 119.23, 100.44, 100.31, 85.80, 85.29, 84.62, 79.81, 79.32, 76.66, 76.14, 74.50, 74.29, 63.12, 62.02, 52.52, 50.14, 49.51, 49.39, 44.06, 43.16, 40.28, 36.50, 31.97, 31.90, 21.76, 19.80. HRMS (m/z) calculated for C₂₅H₂₇ClN₅O₄ [M+H]⁺: 496.1746, found 496.1744.

3-(3-(N-(((1R,2R,3S,4R)-2,3-dihydroxy-4-(7H-pyrrolo[2,3-d]pyrimidin-7-yl)cyclopentyl)methyl)acetamido)prop-1-yn-1-yl)benzamide (5t). To a stirred suspension of 5a (25 mg, 0.048 mmol) and palladium hydroxide on carbon (20% w/w, 9.1mg, 0.013 mmol) in methanol (1 mL) was added ammonium formate (9.8 g. 0.016 mmol). The suspension was heated under reflux for 1 h before filtering through a pad of Celite, eluting with methanol (20 mL). Removal of volatiles and prep HPLC purification afforded 5t as a mixture of rotamers. ¹H NMR (500 MHz, Methanol-d₄) δ 8.93 (d, J=7.2 Hz, 1H), 8.72 (d, J=12.3 Hz, 1H), 7.81-7.69 (m, 2H), 7.68-7.56 (m, 1H), 7.47-7.32 (m, 2H), 6.73-6.65 (m, 1H), 5.20-5.00 (m, 1H), 4.63-4.43 (m, 1H), 4.00-3.91 (m, 1H), 3.76-3.63 (m, 1H), 3.48-3.39 (m, 2H), 2.72 (dt, J=26.4, 7.7 Hz, 2H), 2.40-2.30 (m, 1H), 2.09-2.00 (m, 3H), 1.88-1.70 (m, 1H). ¹³C NMR (126 MHz, MeOD) δ 173.74, 152.06, 151.81, 151.09, 149.86, 149.72, 143.48, 143.04, 135.18, 133.08, 130.59, 129.86, 129.76, 129.60, 128.69, 128.60, 126.50, 126.32, 121.08, 101.20, 101.05, 76.60, 76.06, 74.45, 74.21, 62.41, 61.26, 52.69, 46.87, 44.21, 43.20, 34.00, 33.67, 31.90, 31.84, 31.03, 29.77, 21.84, 21.35. HRMS (m/z) calculated for C₂₄H₂₆N₅O₄ [M+H]⁺: 448.1979, found 448.1979.

3-(3-(N-(((1R,2R,3S,4R)-2,3-dihydroxy-4-(4-(trifluoromethyl)-7H-pyrrolo[2,3-d]pyrimidin-7-yl)cyclopentyl)methyl)acetamido)prop-1-yn-1-yl)benzamide (5u). Compound 5u was prepared according to general procedure E as a mixture of rotamers. ¹H NMR (500 MHz, Methanol-d₄) δ 8.91-8.80 (m, 1H), 7.98-7.92 (m, 1H), 7.91-7.78 (m, 2H), 7.62-7.54 (m, 1H), 7.47-7.37 (m, 1H), 6.81-6.72 (m, 1H), 5.25-5.08 (m, 1H), 4.69-4.54 (m, 1H), 4.53-4.40 (m, 2H), 4.11-3.97 (m, 1H), 3.95-3.76 (m, 1H), 3.75-3.61 (m, 1H), 2.67-2.57 (m, 1H), 2.56-2.41 (m, 1H), 2.30-2.23 (m, 3H), 2.01-1.80 (m, 1H). ¹³C NMR (126 MHz, MeOD) δ 174.10, 173.19, 171.24, 154.30, 151.14, 146.52, 144.40, 135.76, 135.47, 133.25, 132.64, 131.91, 129.86, 129.76, 128.87, 128.61, 124.21, 124.03, 122.02, 116.69, 116.52, 100.04, 99.93, 86.57, 86.01, 84.53, 83.60, 76.62, 76.09, 74.46, 74.24, 62.79, 62.31, 62.15, 61.73, 52.56, 44.08, 43.17, 40.23, 36.46, 31.76, 21.77. HRMS (m/z) calculated for C₂₅H₂₅F₃N₅O₄ [M+H]⁺: 516.1853, found 516.1853.

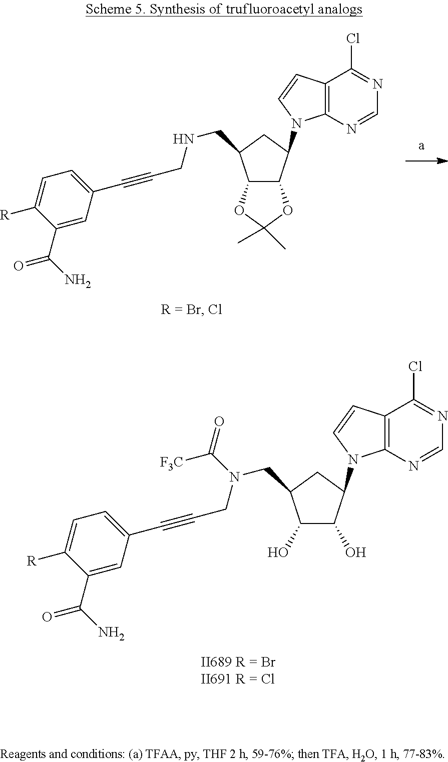

The cellular IC₅₀ for II689 and II691 were 8.5 μM and 3.2 μM, respectively, after 24 hours incubation.

### Example 3 Structure-Activity Relationship (SAR)

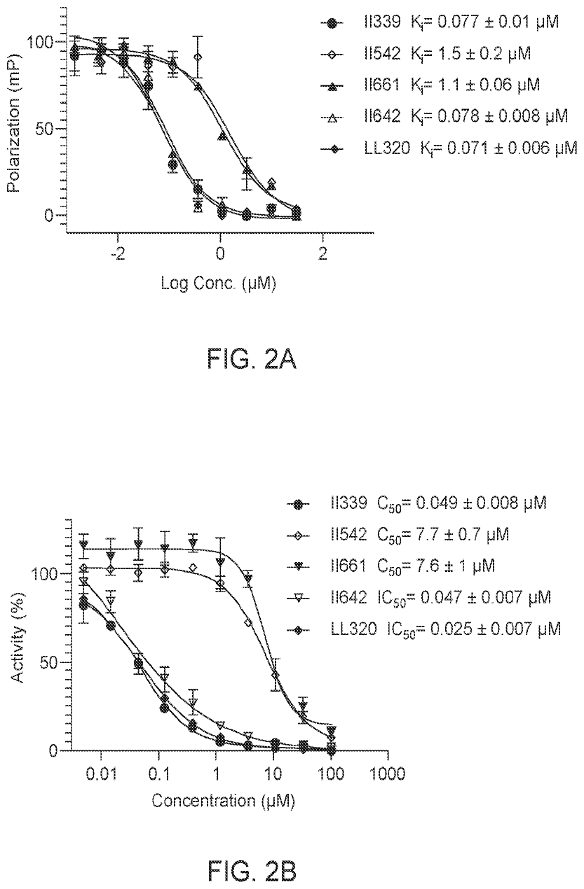

The inhibitory constants (Kᵢ) of the synthesized inhibitors were first determined using a fluorescence polarization (FP)-based competition assay with the previously developed probe II138 as previously reported (Iyamu et al., Anal. Biochem. 2020, 604). Surprisingly, the replacement of the aspartic acid with an acetyl group resulted in a potent bisubstrate inhibitor II399, which displayed a comparable Kᵢ value of 77 nM as our lead compound LL320 (FIG. 2A), whereas II542 containing ethylamine resulted in about a 20-fold reduction in inhibition. As the sensitivity of the FAP assay is limited by the binding affinity of the probe for NNMT, the inhibitory concentration was further determined using an orthogonal S-adenosyl-L-homocysteine hydrolase (SAHH)-coupled fluorescence assay through monitoring the production of SAH (Ivamu et al., RSC Med Chem 12: 1254-1261 (2021); (Mondal et al., Angew Chemie Int Ed 58: 12476-12480 (2019); and Iyamu et al., Anal. Biochem. 2020, 604). The bisubstrate inhibitor II399 retained strong inhibition against NNMT with an IC₅₀ of 49±8 nM like LL320, while II542 only exhibited an IC₅₀ of 7.7 μM. The strong inhibition of II399 against NNMT implied that the aforementioned combinatorial changes including acetyl, 4-chloro pyrrolopyrimidine, and cyclopentane successfully secured the interaction of II399 with NNMT. To understand the contribution of the chloro group, the N⁶ amino analog II642was also synthesized (Scheme 1). Compound II642 displayed an IC₅₀ of 25±7.0 nM in the SAHH-coupled assay. Comparable activities of II399 and II642 supported the feasibility of the chloro substitution for the amino group on the adenosine moiety to maintain the interaction with higher lipophilicity. To examine the contribution from the acetyl group, an acetamide analog of LL320 was also prepared to generate II661 (IC₅₀=7.6 μM). This significant loss implies that the acetyl group alone is not enough but the combination of acetyl group, cyclopentane, and pyrrolopyrimidine together retained the strong interaction of II399 with NNMT.

R₁ substitution. The designed bisubstrate inhibitor II399 with improved lipophilicity retained strong inhibition against NNMT with a Kᵢ of 77±12 nM and IC₅₀ of 49±8 nM with the FP assay and the SAHH-coupled assay respectively (Table 1). The strong inhibitory activity displayed towards NNMT suggests that the acetyl group retains some binding interaction that was present with the aspartic acid in addition to the other binding interactions. Attention was then directed to improving lipophilicity and inhibitory activity by incorporating lipophilic groups onto the acetyl group. Addition of a methyl group afforded II604, which surprisingly has a Kᵢ of 0.87±0.32 μM with the FP assay. With the SAHH coupled assay, the IC₅₀ was 6.7±1.5 μM reflecting about a 136-fold loss of inhibitory activity. This suggests that larger groups may lose inhibitory activity due to steric hindrance with the enzyme. Substituting the acetyl group with glycolic acid II539 retained an inhibitory activity below the sensitivity level of the FP assay but displayed an IC₅₀ of 0.11±0.006 μM with the SAHH coupled assay while the chloromethyl substituent II394 displayed an IC₅₀ of 0.12±0.03 μM, reflecting about a 2-fold loss of inhibitory activity, respectively. Incorporation of a tertiary butyl group II591 to improve lipophilicity further led to a loss of inhibitory activity possibly because of steric effects with an IC₅₀ of 7.5±0.9 μM, which is about 153-fold loss of inhibitory activity. Compound II526, which retains the amino and carboxylic groups of aspartic acid, displayed an IC₅₀ of 0.10±0.007 μM, reflecting only about a 2-fold loss of inhibitory activity in comparison with II399. Cyclization of the amino and carboxylic groups to afford pyrrolidines II544 and II540 displayed a Kᵢ of 20.4±2.8 μM and 3.4±0.6 μM, respectively, with the FP assay. With the SAHH coupled assay, II544 and II540 displayed IC₅₀ of 17.0±1.7 μM and 9.6±2.4 μM, respectively, which were between 346 and 195-fold loss of inhibitory activity, suggesting that a rigid heteroatom is unfavorable. Larger acyclic (II560, II603) and aromatic rings (II556) on the acetyl group also led to loss of inhibitory activity.

TABLE 1
SAR of the acetyl part of the bisubstrate inhibitors

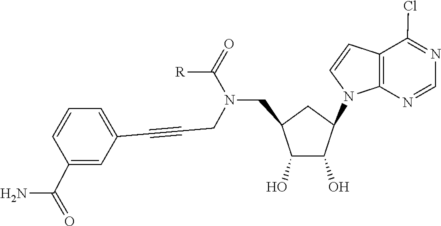

| | | | | Relative | |
|---|---|---|---|---|---|
| | | | SAHH-coupled | potency | |
| | | Kᵢ (FP Assay) | Assay | compared | Permeabilityª |
| Compound | R | (μM) | IC₅₀ (μM) | to II399 | (10⁻⁶ cm/s) |
| II399 5a | 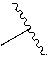 | 0.077 ± 0.012 | 0.049 ± 0.008 | 1 | 0.03 ± 0.005 |
| II604 5b | 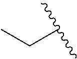 [SMILES: *CC] | 0.87 | 6.7 ± 1.5 | 136 | 0.07 ± 0.01 |
| II539 5c | 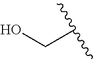 [SMILES: *CO] | 0.17 ± 0.04 | 0.11 ± 0.006 | 2 | ND |
| II394 5d | 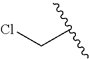 [SMILES: *CCl] | 0.40 ± 0.06 | 0.12 ± 0.03 | 2 | ND |
| II591 5e | 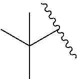 [SMILES: *C(C)(C)C] | | 7.5 ± 0.9 | 153 | 0.05 ± 0.007 |
| II526 5f | 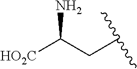 [SMILES: *C[C@H](N)C(=O)O] | 0.019 | 0.10 ± 0.007 | 2 | ND |
| II544 5g | 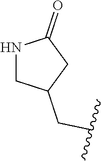 [SMILES: *CC1CNC(=O)C1] | 20.4 ± 2.8 | 17.0 ± 1.7 | 346 | ND |
| II540 5h | 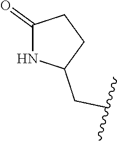 [SMILES: *CC1CCC(=O)N1] | 3.4 ± 0.6 | 9.6 ± 2.4 | 195 | ND |
| II560 5i | 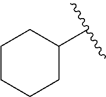 [SMILES: *C1CCCCC1] | 5.4 ± 0.1 | 10.2 ± 3.6 | 208 | 0.05 ± 0.002 |
| II556 5j | 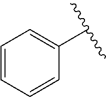 [SMILES: *c1ccccc1] | 18.9 ± 2.3 | 34.8 ± 5.1 | 710 | 0.16 ± 0.007 |
| II603 5k | 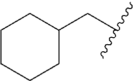 [SMILES: *CC1CCCCC1] | 23.5 ± 1.5 | 24.1 ± 0.01 | 491 | 0.26 ± 0.01 |

^{a}Verapamil was used as the positive control and was 8.6 ± 0.98 in our assay. ND = no detected permability. All experiments were performed in duplicates (n = 2) and presented as mean ± SD.

Benzamide Analogues. To further improve lipophilicity, the amide group on the benzamide was substituted with an acetyl group to afford II590, which lost about 71-fold binding affinity and displayed an IC₅₀ of 3.5±0.2 μM. This demonstrates the importance of the amide group for inhibitory activity. While retaining the benzamide, lipophilic groups were incorporated ortho to the amide to afford compounds II562, II559,and II622 with o-methyl, o-chloro, and o-bromo, respectively. These analogues displayed inhibitory constants well below the sensitivity level of the FP probe, so the SAHH-coupled assay was used for further validation. The lipophilic groups improved the inhibitory activity of II399 between 2 to 8-fold with the p-Cl analogue, II559, the most active, displaying 8-fold improved inhibitory activity over II399 with an IC₅₀ of 0.006±0.0001 μM. The inhibitory activity of II622 was 5-fold more potent than II399 with an IC₅₀ of 0.01±0.005 μM, while II562 with o-methyl was as potent as II399. To further improve the cellular uptake by increased lipophilicity, the benzamide was replaced with naphthalene, which has been previously reported to have a strong binding interaction with NNMT via van der Waals forces. However, substituting the benzamide with 1-naphthalene in II598 resulted in over 347-fold loss in inhibitory function with an IC₅₀ 17.0±1.9 μM and a Kᵢ of 10.5±1.7 μM. The 2-naphthalene analogue also resulted in about 147-fold drop in inhibitory activity displaying an IC₅₀ of 7.2±0.5 μM and Kᵢ of 3.2±0.8 μM in the SAHH-coupled assay and FP assay, respectively. This suggests that substituting the benzamide with a naphthalene moiety is not favorable for the binding of these bisubstrate analogues to NNMT.

Aminoquinoline is a well-studied substrate of NNMT, so it was substituted for the benzamide to evaluate its inhibitory activity. Compound II593 displayed a Kᵢ of 15.2±1.1 μM with the FP assay. In the SAHH coupled assay, II593 displayed an IC₅₀ of 30±0.4 μM, reflecting a 612-fold loss of binding affinity in comparison with II399, which implies unfavorability of incorporated aminoquinoline (Table 2).

TABLE 2
SAR of the benzamide portion of the bisubstrate inhibitors

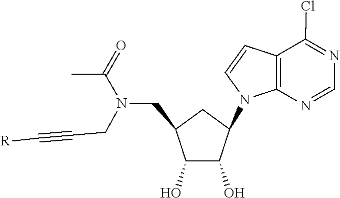

| | | | | Relative | |
|---|---|---|---|---|---|
| | | | SAHH-coupled | Potency | |
| | | Kᵢ (FP Assay) | Assay | compared | Permeabilityª |
| Compound | R | (μM) | IC₅₀ (μM) | to II399 | (10⁻⁶ cm/s) |
| II590 5l | 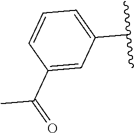 [SMILES: *c1cccc(C(C)=O)c1] | | 3.5 ± 0.2 μM | 71 | 1.4 ± 0.014 |
| II562 5m | 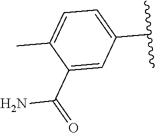 [SMILES: *c1ccc(C)c(C(N)=O)c1] | 0.056 | 0.03 ± 0.001 | 1 | 0.01 ± 0.0007 |
| II559 5n | 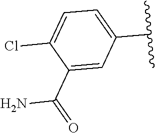 [SMILES: *c1ccc(Cl)c(C(N)=O)c1] | 0.024 | 0.006 ± 0.0001 | 8 | 0.06 ± 0.016 |
| II622 5o | 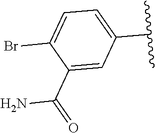 [SMILES: *c1ccc(Br)c(C(N)=O)c1] | | 0.01 ± 0.005 | 5 | 0.07 ± 0.009 |
| II598 5p | 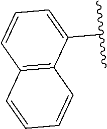 [SMILES: *c1cccc2ccccc12] | 10.5 ± 1.7 | 17.0 ± 1.9 | 347 | 7.6 ± 0.27 |
| II599 5q | 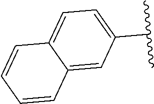 [SMILES: *c1ccc2ccccc2c1] | 3.2 ± 0.8 | 7.2 ± 0.5 | 147 | 1.73 ± 0.20 |
| II593 5r | 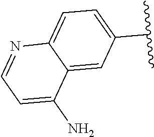 [SMILES: *c1ccc2nccc(N)c2c1] | 15.2 ± 1.1 | 30 ± 0.4 | 612 | ND |

^{a}Verapamil was used as the positive control and was 8.6 ± 0.98 in our assay. ND = no detected permability. All experiments were performed in duplicates (n = 2) and presented as mean ± SD.

The effect of substitution on the benzamide was evaluated. Inhibitors with different halogenated atoms like F and CF₃ at the para position were synthesized. Inhibitors with hydroxyl and methoxy analogs at the para position were also synthesized to investigate the effect of hydrogen bonding. The chloride atom was also substituted at different positions.

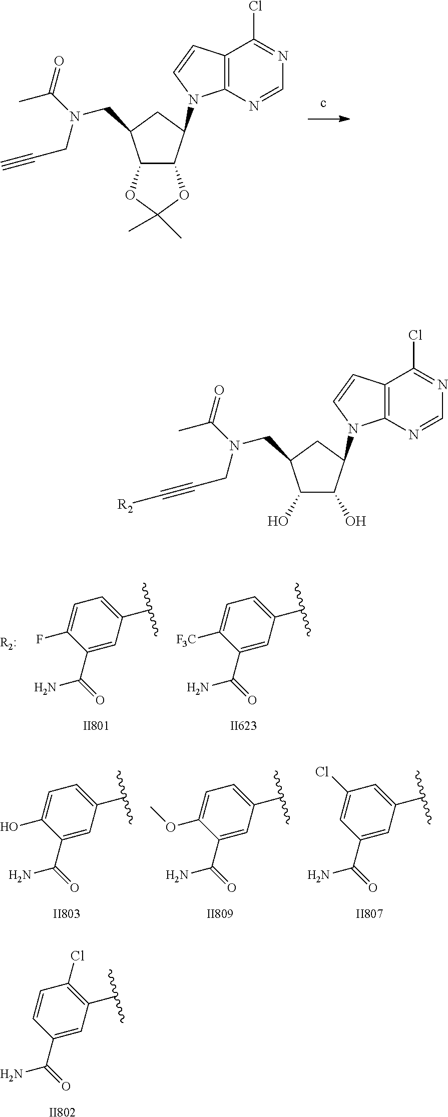

Chloro Adenosine Analogues. To better understand the role of the chloro group in the inhibitory activity, it was substituted with different groups. Substitution of the Cl with H in II643 displayed an IC₅₀ of 1.82±0.04 μM, which reflects about a 37-fold loss of inhibitory function. This suggests that the Cl group contributes to inhibitory activity possibly through halogen bonding. Reports have suggested that chloro substituents on hetero aromatic rings are not very metabolically stable¹⁸, so it was substituted with CF₃ group in compound II582, which displayed an IC₅₀ of 0.420±0.28 μM, which is about 8-fold decreased inhibitory activity in comparison with II399. This suggests that the fluoride did not engage in halogen bonding, which agrees with literature precedent that halogens larger than fluorine form strong halogen bonding with amino acids. To be able to compare the strength of the halo bonding with hydrogen bonding, the chloro substituent was replaced with an amino group in compound II642. Compound II642 displayed an IC₅₀ of 00.025±0.007 μM, which is about 2-fold more potent than II399, suggesting that the strength of the halogen bonding interaction in II399 is similar to the strength of the hydrogen bonding in II642 (Table 3). This indicates that the chloride does not only improve the lipophilicity of the inhibitors but also maintains the binding interaction with NNMT as it maintains a halogen bonding which has the same binding affinity as the hydrogen bonding as the amino substituent.

TABLE 3
SAR of the adenine chloro group on the bisubstrate inhibitors

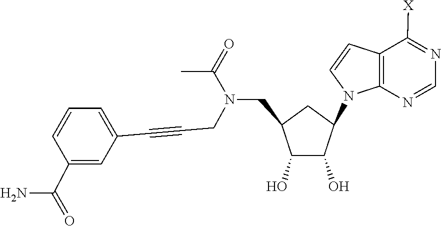

| | | | | Relative | |
|---|---|---|---|---|---|
| | | Kᵢ | SAHH-coupled | Potency | |
| | | (FP Assay) | Assay | compared to | Permeability^{a} |
| Compound | X | (μM) | IC₅₀ (μM) | II399 | (10⁻⁶ cm/s) |
| II582 5s | CF₃ | 0.21 ± 0.04 | 0.420 ± 0.28 | 8 | 0.03 ± 0.001 |
| II643 5t | H | 3.0 ± 0.06 | 1.82 ± 0.04 | 37 | 0.04 ± 0.001 |
| II642 5u | NH₂ | 0.07 ± 0.008 | 0.025 ± 0.007 | 2 | ND |

^{a}Verapamil was used as the positive control and was 8.6 ± 0.98 in our assay. ND = no detected permability. All experiments were performed in duplicates (n = 2) and presented as mean ± SD.

### Example 4 Inhibition Mechanism Studies

To gain an insight into the mode of action of these inhibitors, the most potent inhibitor II559 was selected to investigate the inhibition mechanism by employing kinetic analysis using SAHH-coupled fluorescence assay with recombinant NNMT. The fluorescence-based SAHH-coupled assay was applied to study the IC₅₀ values of compounds to monitor the production of SAH (Iyamu et al., Anal Biochem 604: 113833 (2020); Chen et al., J Med Chem 62: 10783-10797 (2019); and Iyamu et al., Biomolecules 11: 854 (2021)). The assay was performed under the following conditions in a final well volume of 100 μL: 25 mM Tris (pH=7.5), 50 mM KCl, 0.01% Triton X-100, 5 μM SAHH, 0.1 μM NNMT, 10 μM AdoMet, and 10 μM ThioGlo4. After 30 min incubation with inhibitors at 37° C., reactions were initiated by the addition of 10 μM nicotinamide (Km value). The fluorescence signal was monitored on a BMG CLARIOstar microplate reader with excitation 400 nm and emission 465 nm. Data were processed by using GraphPad Prism software 7.0.

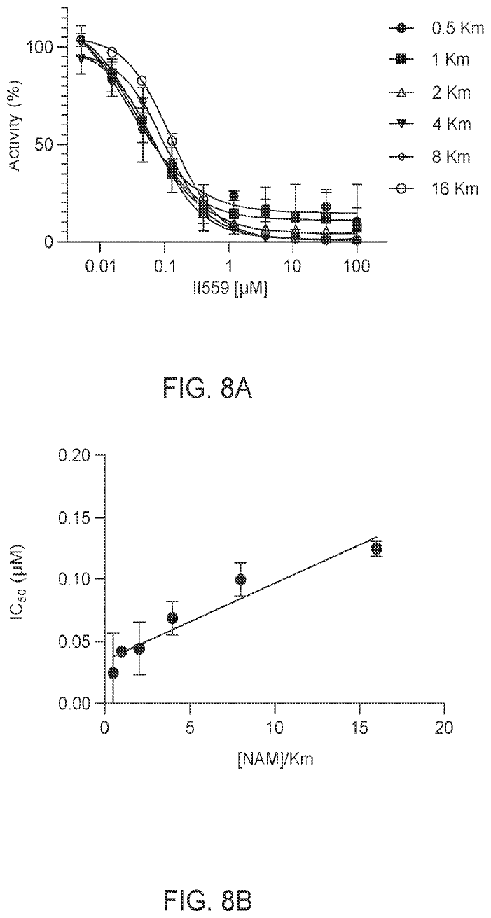

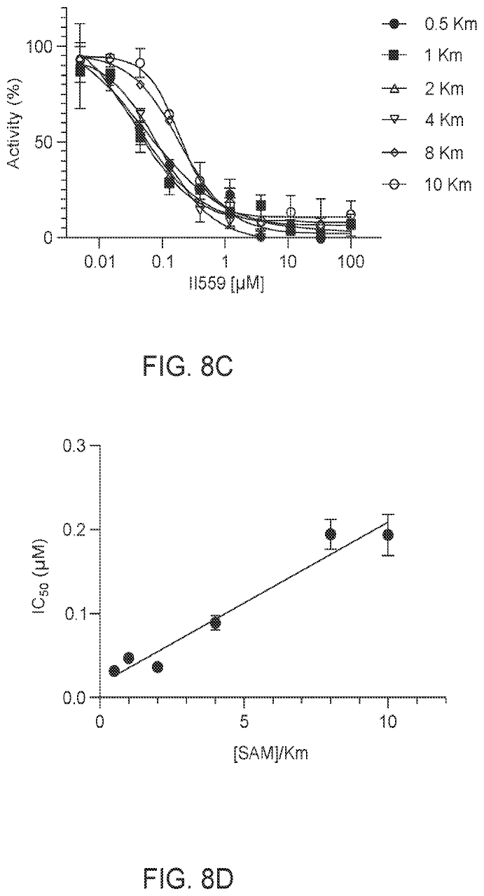

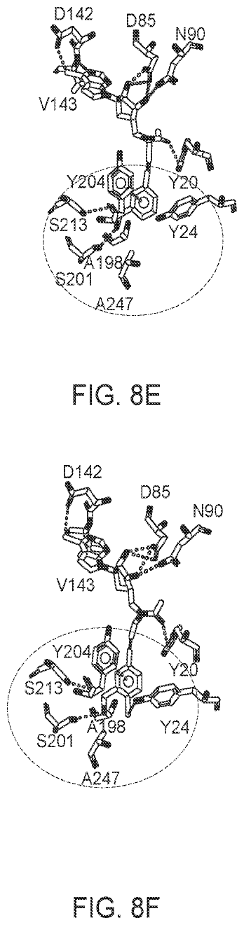

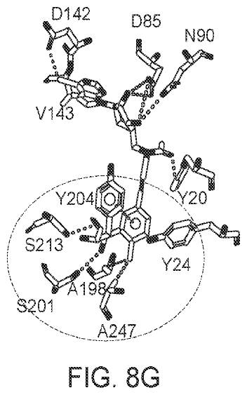

Compound II559 showed an explicit pattern of competitive inhibition for NAM, as demonstrated by a linear increase in the IC₅₀ values in response to the increase of NAM concentration. This inhibitor also displayed an unambiguous pattern of competitive inhibition for SAM. On increasing the SAM concentration, the IC₅₀ values displayed a linear increase in proportion to the increase of SAM. This indicates that II559 is both a SAM and substrate competitive inhibitor (FIGS. 8A-8D). These novel bisubstrate inhibitors, therefore, occupy both the SAM and NAM binding pockets in NNMT.

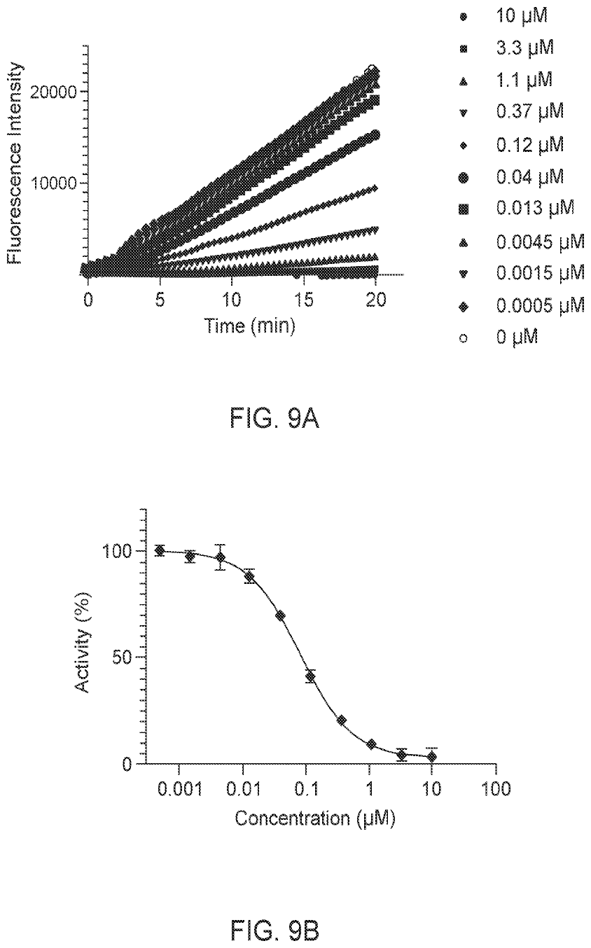

To get the apparent Kᵢ of II559 with a validated mechanism of action, the SAHH-coupled fluorescence assay was performed with SAM and substrate at 4 Kₘ. Compound II559 displayed an apparent Kᵢ value of 1.2±0.04 nM (FIGS. 9A-9B). Its close analogue II562 exhibits an apparent 3.1 nM, assuming its inhibition mechanism is the same.

TABLE 4
NNMT Inhibition

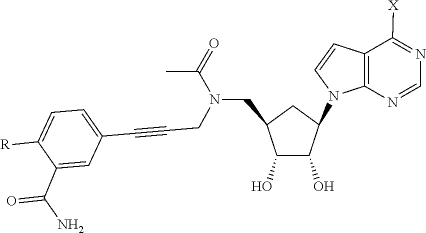

| | | | SAHH-coupled | |
|---|---|---|---|---|
| | | | Assay | |
| Compound | X | R | IC₅₀ (μM)^{a} | Kᵢ, app (nM)^{b} |
| II399 | Cl | H | 0.15 ± 0.05ª | 5.9 ± 0.5 |
| II562 | Cl | CH₃ | 0.08 ± 0.02ª | 3.1 ± 0.9 |
| II559 | Cl | Cl | 0.026 ± 0.006ª | 1.2 ± 0.04 |
| II622 | Cl | Br | 0.05 ± 0.01ª | 2 ± 0.07 |
| II642 | NH₂ | H | 0.093 ± 0.007^{a} | 4.0 ± 0.02 |

^{a}These experiments were performed with substrate and SAM concentration at 4 Kₘ as the IC₅₀ at Kₘ value was less than the enzyme concentration. ^{b}K_{i, app} was calculated using the equation K_{i, app} = IC₅₀/(1+[S]/Kₘ). All experiments were performed in duplicates (n = 2) and presented as mean ± SD.

After establishing the mechanism of action of II559 as a bisubstrate inhibitor, the SAHH-coupled assay of the more potent inhibitors was performed at 4 Kₘ concentration of both the substrate and SAM because their IC₅₀ values were less than the enzyme concentration. They all showed inhibitory constants in the single-digit nM range with II399 displaying a K_{i, app} of 5.9±0.5 nM and II559 displaying the best inhibitory activity with a K_{i, app} of 1.2±0.04 nM.

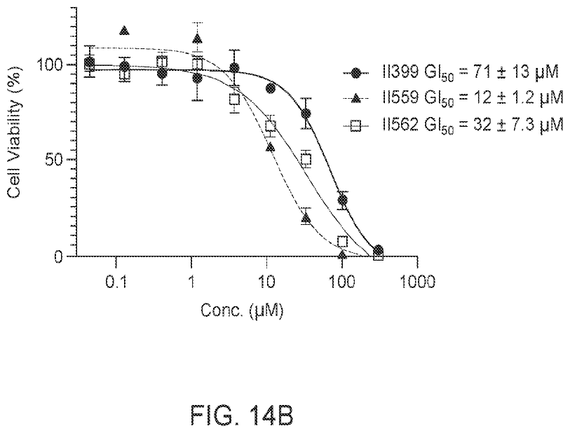

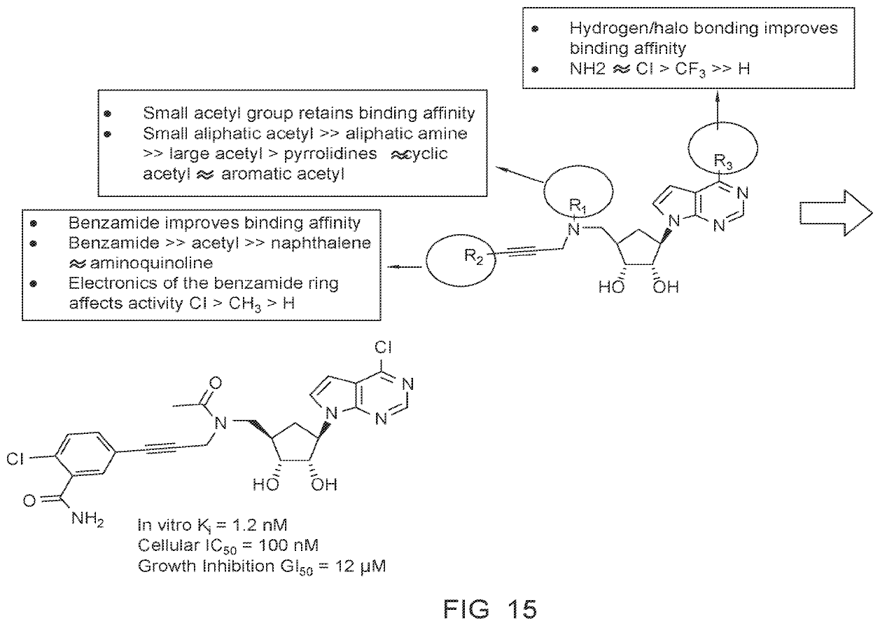

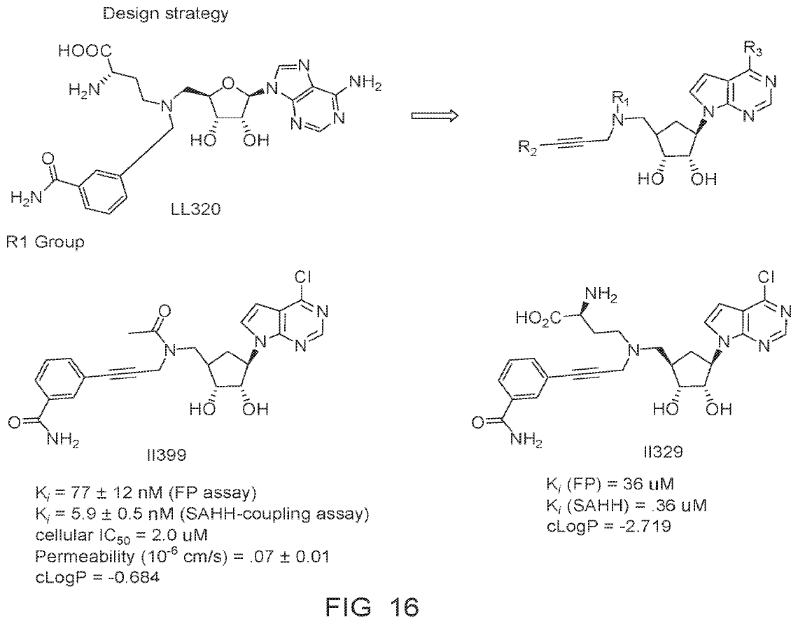

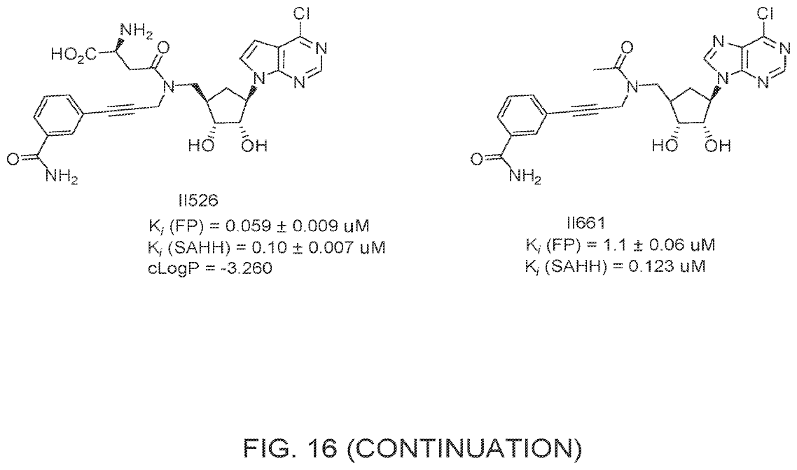

II559 and II562 displayed more potent inhibition than II399, with apparent IC₅₀ values below the sensitivity of the assay at Kₘ condition of both SAM and NAM. To obtain a relatively accurate apparent IC₅₀ value, their inhibition was re-characterized under the concentrations of both SAM and substrate at their 9 Kₘ values in the SAHH-coupled fluorescence assay (FIG. 14A). II559 and II562 displayed apparent IC₅₀ values of 120 nM and 310 nM at 9 Kₘ conditions, respectively. Accordingly, the apparent Kᵢ values of II559 and II562 were then calculated to be 1.2 and 3.1 nM, respectively, according to the equation Kᵢ=IC₅₀/(1+[S]/Kₘ).

### Example 5 Cell Membrane Permeability

The ability of these new and more lipophilic analogues to cross the cell membrane was evaluated in the renal cancer cell line 769P. The 769P cell line was cultured in RPMI medium supplemented with 10% fetal bovine serum (Gibco). The cells were cultured in a tissue culture dish (Falcon 353003). Cells were maintained in cell culture flasks until seeding into a 12-well tissue culture plate (Falcon 353047). Medium was removed, and the cells were washed with DPBS (1 mL) twice followed by treatment with TrypLE Express (1 mL) into a 100×20 mm culture flask. The reaction was quenched by the addition of 4 mL of media, and the cells were counted. Cells were seeded into a 12-well tissue culture plate (Falcon 353047) at a density of 0.1×10⁶ cells/mL and incubated overnight at 37° C., 5% CO₂, and 95% humidity with the lid on. They were then treated with the inhibitors at different concentrations, and incubation was continued for the specified time (5 hours). The medium was removed, and the cells were washed with 1×PBS three times to remove any residual compound or peptide attached to the cell surface. Then, 100 μL of 1×PBS were added, and the cells were snap-frozen in liquid nitrogen twice. The cell lysate was then analyzed with MALDI using DHB matrix to identify the presence of the compound inside the cell. After incubation of II399 with the cells for 5 hours and analyzing the cell lysate by MALDI-TOF, there was evidence of II399 in the cell lysate at 50, 5, and 1 μM concentration validating the ability of these bisubstrate inhibitors to cross the cell membrane (Table 5).

TABLE 5
Cell Permeability of Inhibitors

| | | Permeabilityª |
|---|---|---|
| Compound | Structure | (10⁻⁶ cm/s) |
| 5a (II399) | 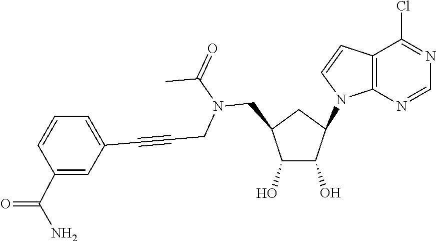 [SMILES: CC(=O)N(CC#Cc1cccc(C(N)=O)c1)C[C@H]1C[C@@H](n2ccc3c(Cl)ncnc32)[C@H](O)[C@@H]1O] | 0.3 ± 0.1 |
| 5b | 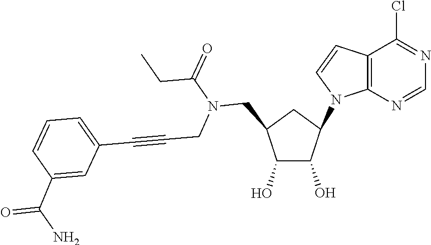 [SMILES: CCC(=O)N(CC#Cc1cccc(C(N)=O)c1)C[C@H]1C[C@@H](n2ccc3c(Cl)ncnc32)[C@H](O)[C@@H]1O] | 0.4 ± 0.1 |
| 5c | 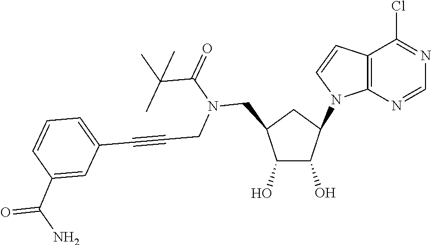 [SMILES: CC(C)(C)C(=O)N(CC#Cc1cccc(C(N)=O)c1)C[C@H]1C[C@@H](n2ccc3c(Cl)ncnc32)[C@H](O)[C@@H]1O] | 0.5 ± 0.1 |
| 5d | 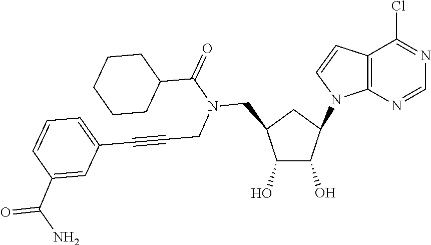 [SMILES: NC(=O)c1cccc(C#CCN(C[C@H]2C[C@@H](n3ccc4c(Cl)ncnc43)[C@H](O)[C@@H]2O)C(=O)C2CCCCC2)c1] | 0.5 ± 0.1 |
| 5e | 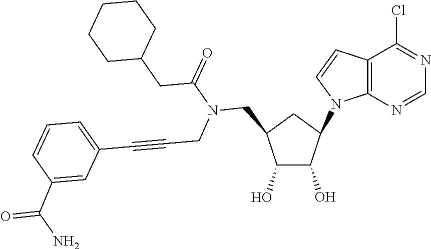 [SMILES: NC(=O)c1cccc(C#CCN(C[C@H]2C[C@@H](n3ccc4c(Cl)ncnc43)[C@H](O)[C@@H]2O)C(=O)CC2CCCCC2)c1] | 0.6 ± 0.1 |
| 5f | 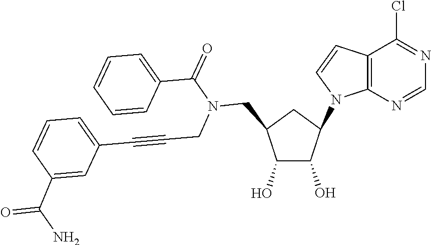 [SMILES: NC(=O)c1cccc(C#CCN(C[C@H]2C[C@@H](n3ccc4c(Cl)ncnc43)[C@H](O)[C@@H]2O)C(=O)c2ccccc2)c1] | 0.6 ± 0.1 |
| 5g | 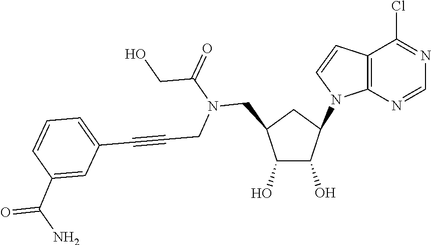 [SMILES: NC(=O)c1cccc(C#CCN(C[C@H]2C[C@@H](n3ccc4c(Cl)ncnc43)[C@H](O)[C@@H]2O)C(=O)CO)c1] | ND |
| 5h | 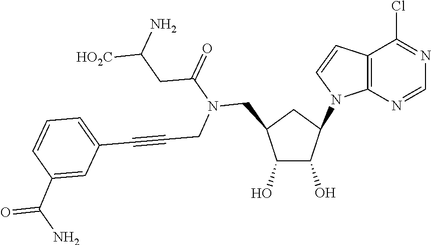 [SMILES: NC(=O)c1cccc(C#CCN(C[C@H]2C[C@@H](n3ccc4c(Cl)ncnc43)[C@H](O)[C@@H]2O)C(=O)CC(N)C(=O)O)c1] | ND |
| 5i | 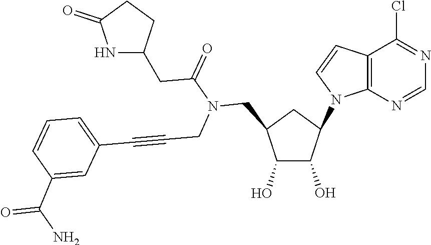 [SMILES: NC(=O)c1cccc(C#CCN(C[C@H]2C[C@@H](n3ccc4c(Cl)ncnc43)[C@H](O)[C@@H]2O)C(=O)CC2CCC(=O)N2)c1] | ND |
| 5j | 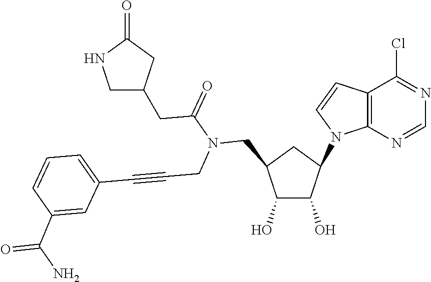 [SMILES: NC(=O)c1cccc(C#CCN(C[C@H]2C[C@@H](n3ccc4c(Cl)ncnc43)[C@H](O)[C@@H]2O)C(=O)CC2CNC(=O)C2)c1] | ND |
| 5k | 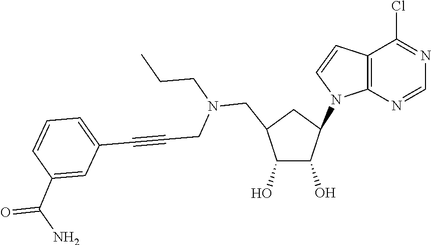 [SMILES: CCCN(CC#Cc1cccc(C(N)=O)c1)CC1C[C@@H](n2ccc3c(Cl)ncnc32)[C@H](O)[C@@H]1O] | ND |
| 5l | 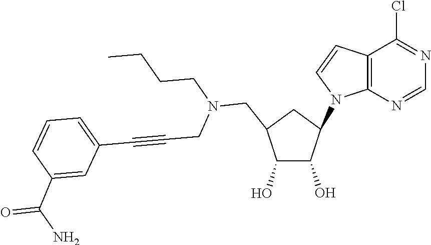 [SMILES: CCCCN(CC#Cc1cccc(C(N)=O)c1)CC1C[C@@H](n2ccc3c(Cl)ncnc32)[C@H](O)[C@@H]1O] | ND |
| 5m | 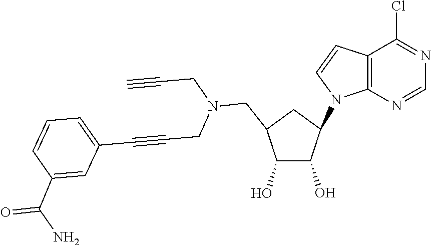 [SMILES: C#CCN(CC#Cc1cccc(C(N)=O)c1)CC1C[C@@H](n2ccc3c(Cl)ncnc32)[C@H](O)[C@@H]1O] | ND |
| 5n | 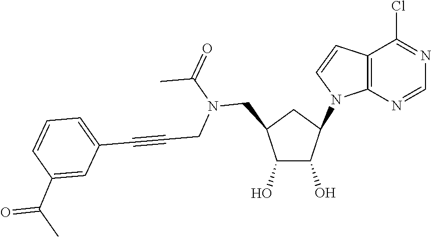 [SMILES: CC(=O)c1cccc(C#CCN(C[C@H]2C[C@@H](n3ccc4c(Cl)ncnc43)[C@H](O)[C@@H]2O)C(C)=O)c1] | 1.4 ± 0.2 |
| 5o | 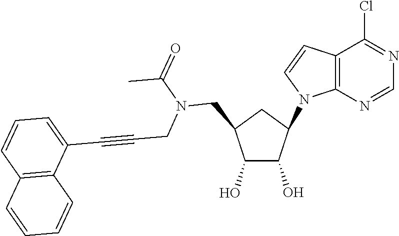 [SMILES: CC(=O)N(CC#Cc1cccc2ccccc12)C[C@H]1C[C@@H](n2ccc3c(Cl)ncnc32)[C@H](O)[C@@H]1O] | 7.6 ± 0.3 |
| 5p | 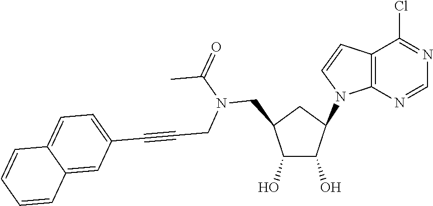 [SMILES: CC(=O)N(CC#Cc1ccc2ccccc2c1)C[C@H]1C[C@@H](n2ccc3c(Cl)ncnc32)[C@H](O)[C@@H]1O] | 2.1 ± 0.2 |
| 5q | 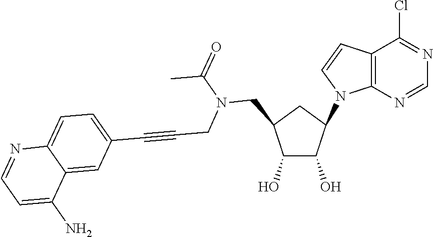 [SMILES: CC(=O)N(CC#Cc1ccc2nccc(N)c2c1)C[C@H]1C[C@@H](n2ccc3c(Cl)ncnc32)[C@H](O)[C@@H]1O] | ND |
| 5r | 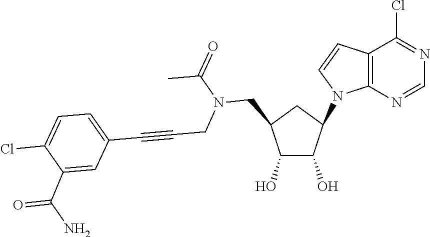 [SMILES: CC(=O)N(CC#Cc1ccc(Cl)c(C(N)=O)c1)C[C@H]1C[C@@H](n2ccc3c(Cl)ncnc32)[C@H](O)[C@@H]1O] | 0.5 ± 0.1 |
| 5s | 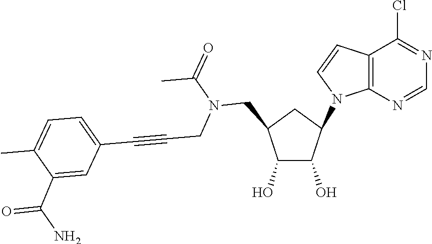 [SMILES: CC(=O)N(CC#Cc1ccc(C)c(C(N)=O)c1)C[C@H]1C[C@@H](n2ccc3c(Cl)ncnc32)[C@H](O)[C@@H]1O] | 0.6 ± 0.2 |
| 5t | 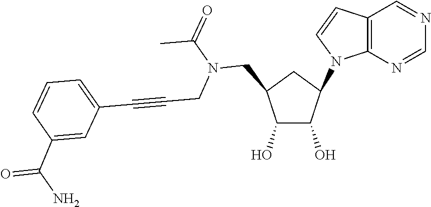 [SMILES: CC(=O)N(CC#Cc1cccc(C(N)=O)c1)C[C@H]1C[C@@H](n2ccc3cncnc32)[C@H](O)[C@@H]1O] | 0.4 ± 0.1 |
| 5u | 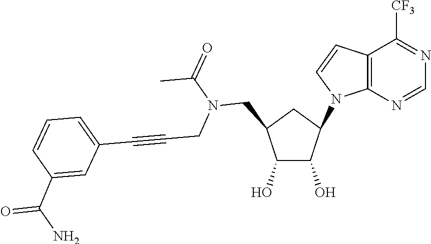 [SMILES: CC(=O)N(CC#Cc1cccc(C(N)=O)c1)C[C@H]1C[C@@H](n2ccc3c(C(F)(F)F)ncnc32)[C@H](O)[C@@H]1O] | 0.3 ± 0.1 |

^{a}Verapamil, which was used as the positive control, was determined to have a permeability value of 8.6 ± 0.98 × 10⁻⁶ cm/s, which is comparable to the literature value (8.8 ± 0.53 × 10⁻⁶ cm/s; Chen et al., Pharm Res 25(7): 1511-1520 (2008)). ND = no detected permeability. All experiments were performed in duplicate (n = 2) and are presented as mean ± SD.

### Example 6 Artificial Membrane Permeability

Parallel Artificial Membrane Permeability Assay (PAMPA) was then employed to evaluate the permeability of these inhibitors using verapamil as a control. The majority of these analogues showed limited ability to cross the cell membrane. Compound II399, for example, displayed a permeability value of 0.03±0.005×10⁻⁶ cm/s. The addition of lipophilic groups on the acetyl substitution only moderately improved the membrane permeability as the methyl cyclohexane II603 had a permeability value of 0.26±0.01×10⁻⁶ cm/s. Replacement of the benzamide with acetophenone in II590 had a much improved permeability of 1.4±0.014×10⁻⁶ cm/s suggesting that masking the benzamide with a prodrug of substituting with lipophilic groups improves cellular uptake. Incorporating lipophilic groups (Me, Cl, Br) on the ortho position of the benzamide only moderately improved membrane permeability with II559 displaying a permeability value of 0.06±0.016×10⁻⁶ cm/s. The compounds with the best membrane permeability values were the analogues that substituted benzamide for naphthalene with o- and p-naphthalene displaying membrane permeability of 7.6±0.27×10⁻⁶ cm/s and 1.73±0.20×10⁻⁶ cm/s, respectively. Analogues with hydrophilic groups like II539, II526, II554, II540, and II593 did not display any ability to cross the artificial membrane.

### Example 7 Cellular Target Engagement

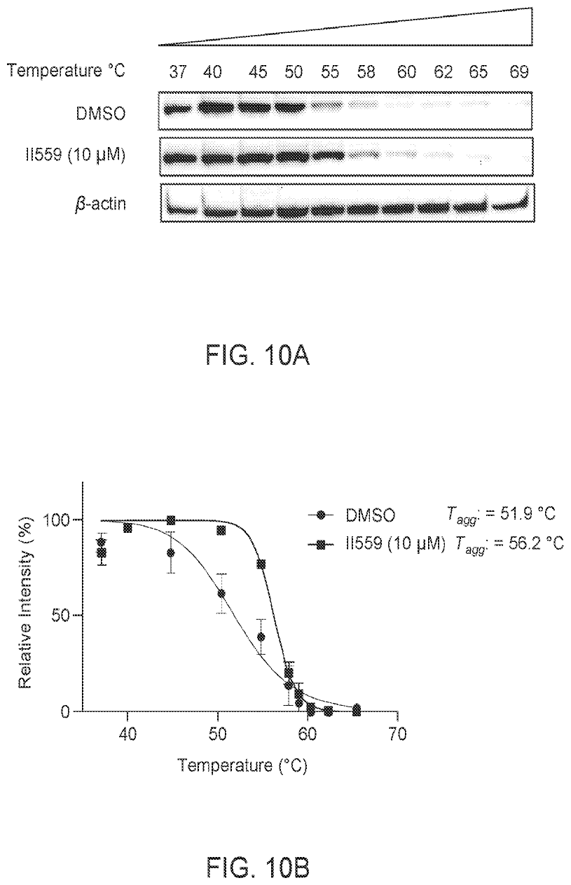

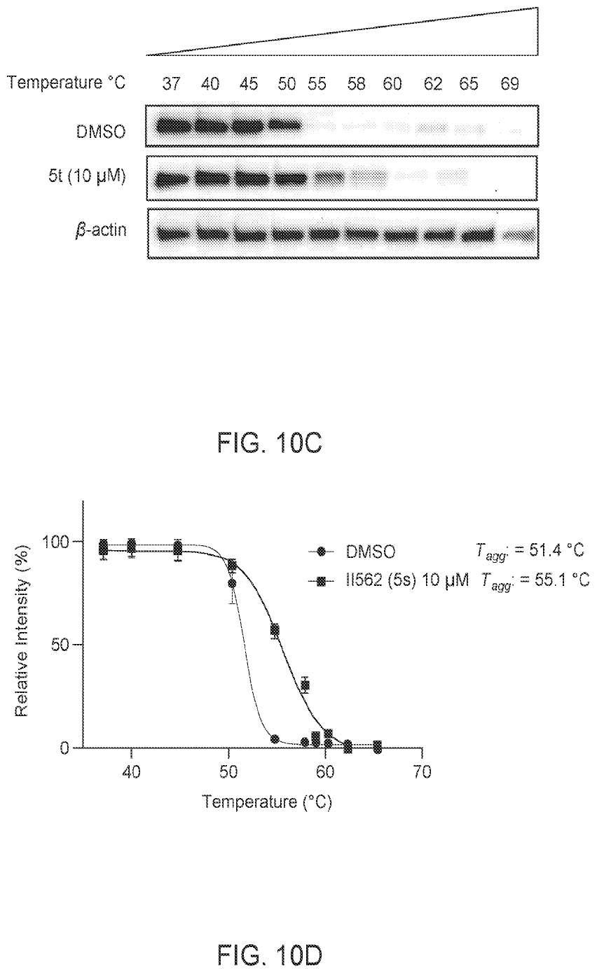

Bisubstrate inhibitors are generally not cell potent due to their properties. There have been attempts to incorporate lipophilic prodrug handles to improve their cellular uptake (Iyamu et al., Biomolecules 11: 854 (2021); Alston et al., Arch Biochem Biophys 260: 601-608 (1988); and Ulanovskaya et al., Nat Chem Biol 9: 300-306 (2013)). The ability of the most potent lipophilic bisubstrate inhibitor (II559) to engage NNMT upon entering the cell was evaluated. After 4 hours of incubation of II559 (10 μM) with 769P cells, which have been shown to overexpress NNMT, the bisubstrate inhibitor was able to cause a higher thermal stabilization (Tagg=4.3° C.) (see FIGS. 10A-10B). This clearly demonstrated that II559 can engage with NNMT upon entering the cell.

The cellular target engagement of II559 and II562 was evaluated using cellular thermal shift assay (Jafari et al., Nat Proc 9(9): 2100-2122 (2014)). Human renal cancer cell line 769P was selected because of its high expression level of NNMT (Ulanovskaya et al., Nat Chem Biol 9(5): 300-306 (2013)). The assay was performed based on a modified protocol for the cell lysate CETSA experiments (Jafari et al. (2014), supra). Briefly, renal cell lines were cultured in RPMI media supplemented with 10% FBS (Gibco). The cells were maintained in a tissue culture dish (Falcon 353003) until seeding at a density of 2.0×10⁶ cells/mL into two 100 mm tissue culture dishes (Falcon 353003). The cells were incubated overnight at 37° C., 5% CO₂ with the lid on. One culture dish was treated with 10 μM of the inhibitor for a total 1% DMSO in the media. The control culture dish was treated with only DMSO. The cells were then incubated for the specified time (4 hours). After incubation, the cells were harvested and washed with PBS and finally resuspended in 1 mL PBS. Aliquots of 100 μL from each condition were distributed into PCR strip tubes and heated at 37, 40, 45, 50, 55, 58, 60, 62, 65, and 69° C. for 3 minutes before being cooled to room temperature in 3 minutes. The cells were lysed by three freeze-thaw cycles with liquid nitrogen and clarified by centrifugation at 20,000 g for 20 minutes at 4° C. The soluble fractions (lysate) were then analyzed by immunoblotting using the anti-NNMT antibody (Proteintech 15123-1-AP) and quantified using Image J (Wu et al., PLoS One 5(1) (2010)).

At a concentration of 10 μM, II559 and II562 enhanced the thermal stabilization of NNMT with a ΔTagg value of 4.3° C. and 3.7° C., respectively (FIGS. 10C-10D). Notably, II559 induced higher thermal stability of NNMT than II562, displaying a consistent trend as their inhibitory activities and binding affinities. The increased cellular thermal stability of NNMT confirmed the cellular engagement of both II559 and II562 with NNMT.

### Example 8 Selectivity Studies

Bisubstrate inhibitors have been reported to be highly selective to their target. To investigate the selectivity of this class of bisubstrate inhibitors, the inhibitory activity of II559 was evaluated against a panel of methyltransferases including phenylethanolamine N-methyltransferase (PNMT) and indoleethylamine N-methyltransferase (INMT), which are small molecule methyltransferase closely related to NNMT, two enzymes (G9a and SETD7) from the protein lysine methyltransferase PKMT, and two members from the protein arginine methyltransferase PRMT (PRMT1 and ThPRMT7). NTMT1, which shares a similar co-factor with NNMT, was also included in addition to SAHH because it also has a SAH binding site and is employed in the coupled fluorescence assay.

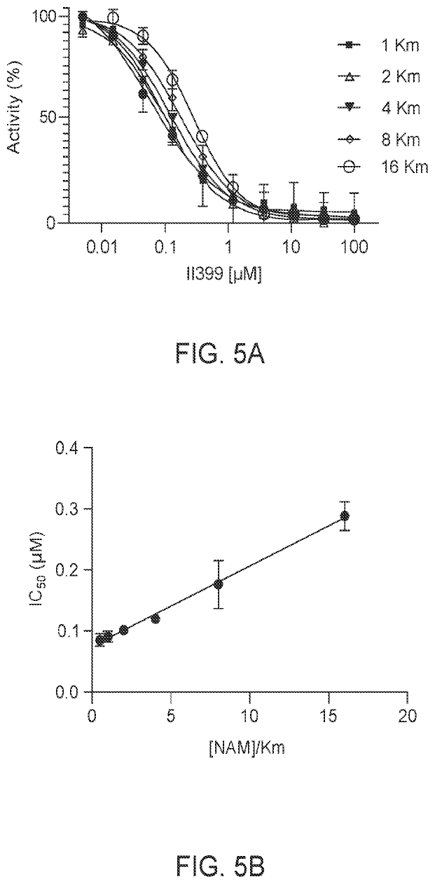

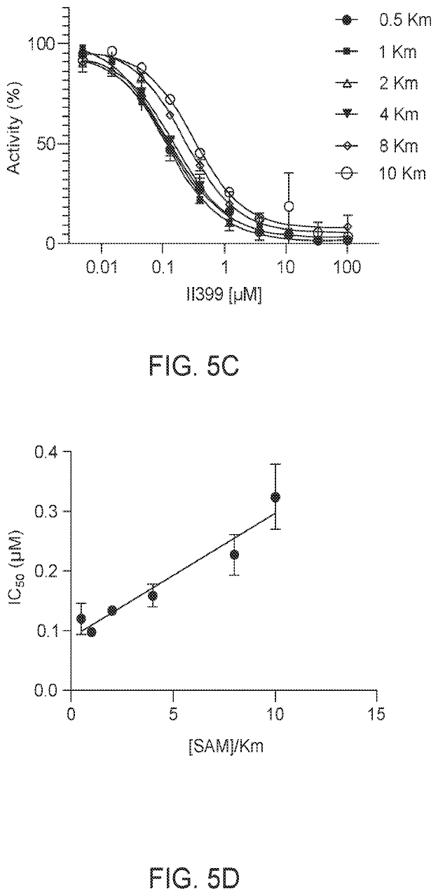

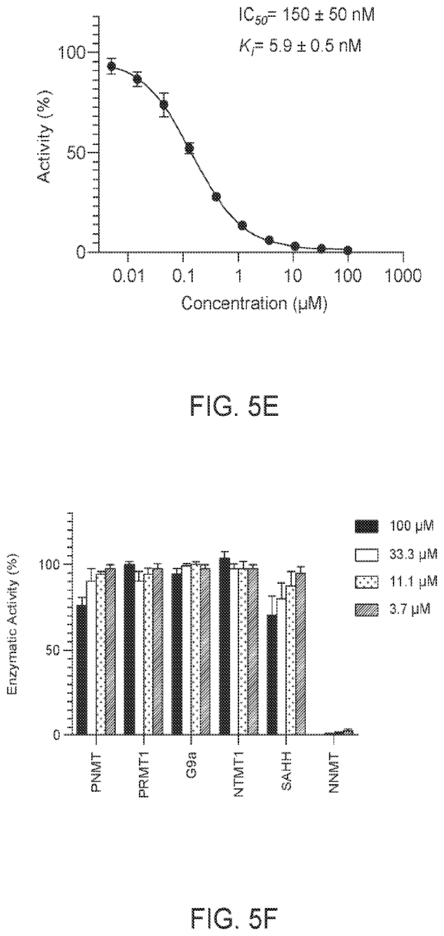

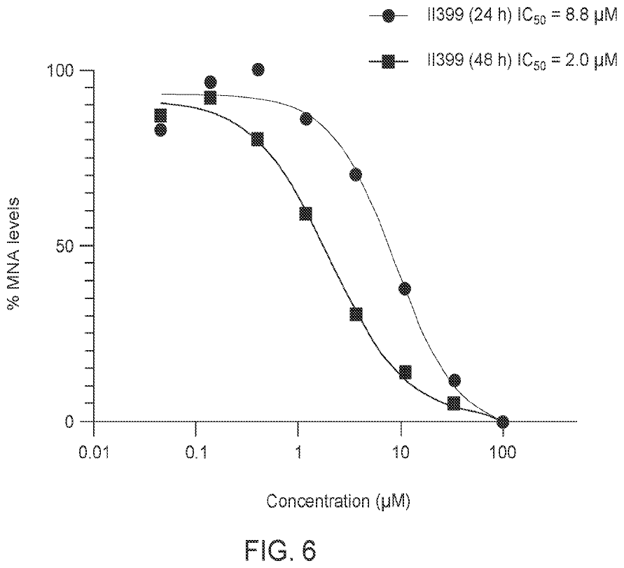

To examine the mode of action of II399, the inhibition mechanism studies were performed using SAHH-coupled fluorescence assay with NNMT. Compound II399showed an explicit pattern of competitive inhibition for NAM, as demonstrated by a linear increase in the IC₅₀ values in response to the increase of NAM concentration (FIGS. 5A and 5B). Similar trends were observed when the concentration of SAM was increased (FIGS. 5C and 5D). These results indicated that II399 is both a SAM and substrate competitive inhibitor, occupying both the SAM and NAM binding pockets of NNMT in a reversible manner. With a confirmed inhibition mechanism, we determined the Kᵢ value of II399 to be 5.9±0.5 nM as II399 displayed an IC₅₀ value of 150±50 nM with both SAM and substrate concentrations at 4 Kₘ (FIG. 5E). Next, the selectivity of II399 for NNMT was assessed against a panel of SAM-dependent methyltransferases, including closely related PNMT, PRMT1, NTMT1, and protein lysine methyltransferase G9a. In addition, the coupling enzyme SAHH was also included because it has a SAH binding site. Remarkably, II399 is over 1,000-fold more selective than the aforementioned enzymes and showed minimal inhibition at 100 μM concentration (FIG. 5F). Notably, II399 displays a higher selectivity than LL320 for both NTMT1 and SAHH while maintaining comparable selectivity for PNMT, PRMT1, and G9a (Chen et al., J. Med. Chem. 62: 10783-10797 (2019)).

A fluorescence-based SAHH-coupled assay was applied to study the inhibitory effect of II559 on methyltransferase activity of PNMT, G9a, PRMT1, NTMT1, and SAHH. For PNMT, the assay was performed in a final well volume of 100 μL: 25 mM potassium phosphate buffer (pH=7.6), 1 mM EDTA, 2 mM MgCl₂, 0.01% Triton X-100, 5 μM SAHH, 0.2 μM PNMT, 3 μM AdoMet, and 10 μM ThioGlo4. The inhibitor was added at four compound concentrations: 100, 33.3, 11.1, and 3.7 μM. After 10 minutes incubation with the inhibitor, reactions were initiated by the addition of 40 μM norepinephrine.

For G9a, the assay was performed in a final well volume of 100 μL: 25 mM potassium phosphate buffer (pH=7.6), 1 mM EDTA, 2 mM MgCl₂, 0.01% Triton X-100, 5 μM SAHH, 0.1 μM His-G9a, 10 μM AdoMet, and 10 μM ThioGlo4. The inhibitor was added at four compound concentrations: 100, 33.3, 11.1, and 3.7 μM. After 10 minutes incubation with the inhibitor, reactions were initiated by the addition of 4 μM H3-21 peptide.

For NTMT1, the assay was performed in a final well volume of 100 μL: 25 mM Tris (pH=7.5), 50 mM KCl, 0.01% Triton X-100, 5 μM SAHH, 0.1 μM NTMT1, 3 μM AdoMet, and 10 μM ThioGlo4. After 10 minutes incubation with the inhibitor, reactions were initiated by the addition of 0.5 μM GPKRIA peptide.

For SETD7, the assay was performed in a final well volume of 100 μL: 25 mM potassium phosphate buffer (pH=7.6), 0.01% Triton X-100, 5 μM SAHH, 1 μM His-SETD7, 2 μM AdoMet, and 15 μM ThioGlo1. After 10 minutes incubation with the inhibitor, reactions were initiated by the addition of 90 μM H3-21 peptide.

For PRMT1, the assay was also performed in a final well volume of 100 μL: 2.5 mM HEPES (pH=7.0), 25 mM NaCl, 25 μM EDTA, 50 μM TCEP, 0.01% Triton X-100, 5 μM SAHH, 0.2 μM PRMT1, 10 μM AdoMet, and 15 μM ThioGlol. After 10 minutes incubation with the inhibitor, reactions were initiated by the addition of 5 μM H4-21 peptide.

For TbPRMT7, the assay was performed in a final well volume of 100 μL: 25 mM Tris (pH=7.5), 50 mM KCl, 0.01% Triton X-100, 5 μM SAHH, 0.2 μM TbPRMT7, 3 μM AdoMet, and 15 μM ThioGlo1. After 10 minutes incubation with the inhibitor, reactions were initiated by the addition of 60 μM H4-21 peptide.

All experiments were determined in duplicate. Fluorescence was monitored on a BMG CLARIOstar microplate reader with excitation 380 nm and emission 505 nm.

The compound was evaluated for its effect on SAHH activity, the coupled enzyme. The assay was performed in a final well volume of 100 μL: 25 mM Tris (pH=7.5), 50 mM KCl, 0.01% Triton X-100, 0.1 μM SAHH, and 15 μM ThioGlol. After 10 min incubation with the compound, 0.5 μM SAH was added to initiate the reactions. The experiment was determined in duplicate. Fluorescence was monitored on a BMG CLARIOstar microplate reader with excitation 380 nm and emission 505 nm.

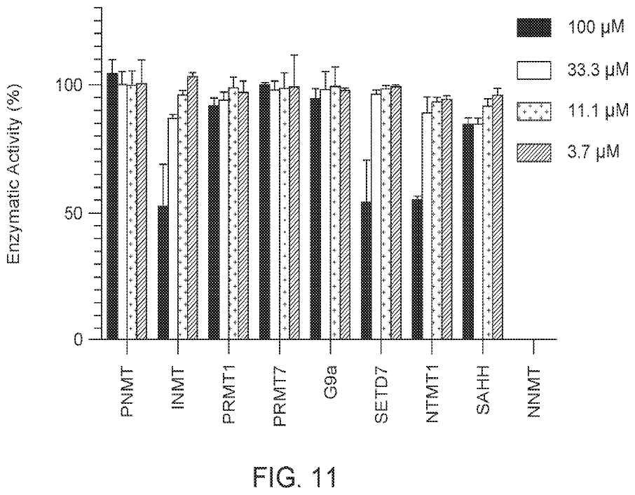

As shown in FIG. 11, II559 is over 10,000-fold more selective over SAM-dependent protein methyltransferases including PRMT1, PRMT7, and G9a as it barely showed any inhibition at 100 μM concentration. Although at 100 μM concentration, II559 inhibits about 40% of INMT activity, it was also over 10,000-fold more selective for PNMT with which it shares about 39% sequence identity. At 100 μM, compound II559 inhibited 45% of NTMTI activity and about 38% of the activity of SETD7. Overall, this novel bisubstrate inhibitor displays better selectivity in comparison with LL320.

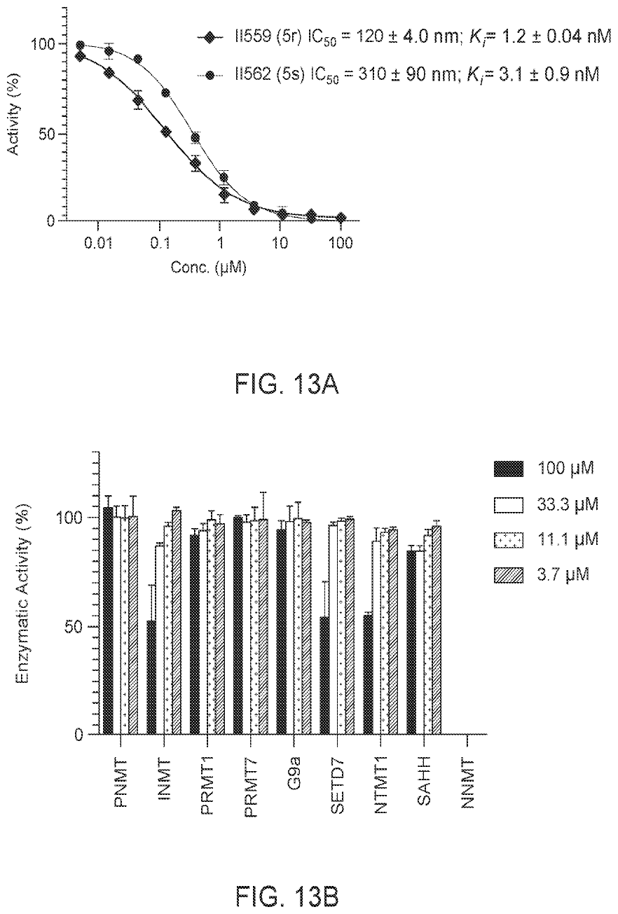

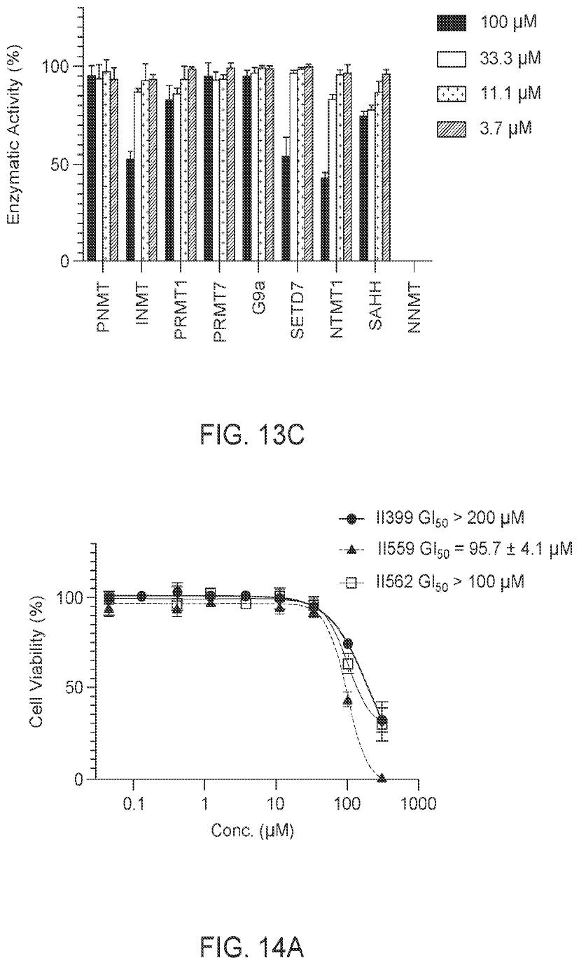

To investigate the selectivity of this class of bisubstrate inhibitors, the inhibitory activity of II559 and II562 were evaluated against a panel of methyltransferases including phenylethanolamine N-methyltransferase (PNMT) and indoleethylamine N-methyltransferase (INMT) which are small molecule methyltransferase closely related to NNMT, two enzymes (G9a and SETD7) from the protein lysine methyltransferase PKMT, and two members from the protein arginine methyltransferase PRMT (PRMT1 and TbPRMT7) (Riss et al., Assay Guid Man 1-25 (2013)). NTMT1 which shares a similar co-factor with NNMT was also included in addition to SAHH because it also has a SAH binding site and is employed in the coupled fluorescence assay. As shown in FIG. 13B, II559 is over 5,000-fold more selective for NNMT over two closely-related methyltransferases INMT and PNMT by only inhibiting 40% of INMT activity and no detectable inhibition for PNMT at 100 μM. Similar selectivity was also observed for NTMT1 and SETD7 as II559 inhibited 45% of NTMT1 activity and about 38% of the activity of SETD7 at 100 μM. Higher selectivity was achieved for other SAM-dependent protein methyltransferases including PRMT1, PRMT7, G9a as II559 barely showed any inhibition at 100 μM concentration. Like II559, II562 also displayed a similar selectivity profile with over 5,000-fold more selective for NNMT compared to PRMT1, PRMT7 and G9a, although it inhibited about 60% of NTMT1 activity and about 45% of the activity of SETD7 at 100 μM (see FIG. 13C). Overall, these novel bisubstrate inhibitors display slightly improved or comparable selectivity in comparison with II399.

### Example 9 Co-Crystal Structure of II339 in Complex with NNMT

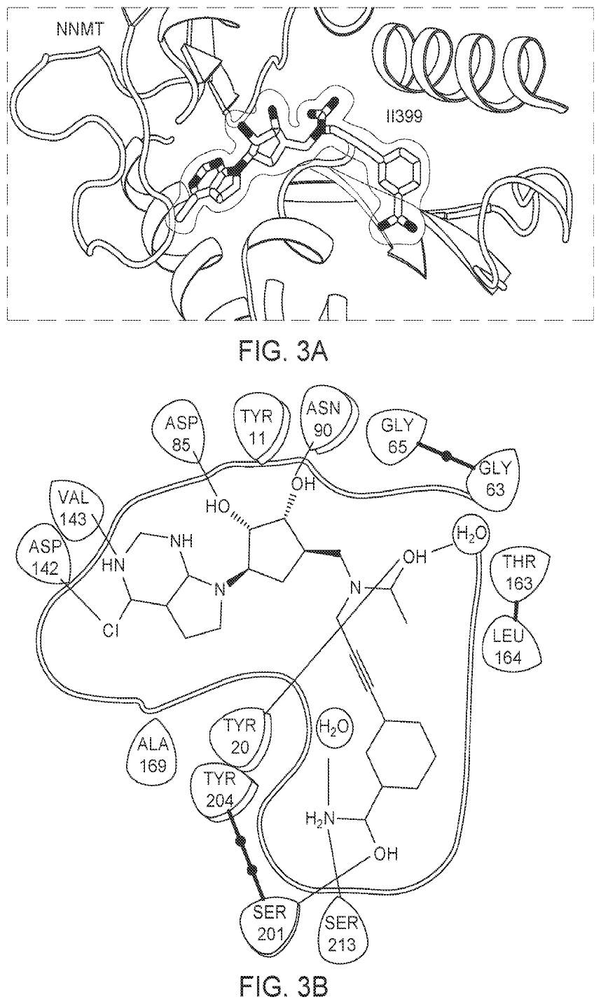

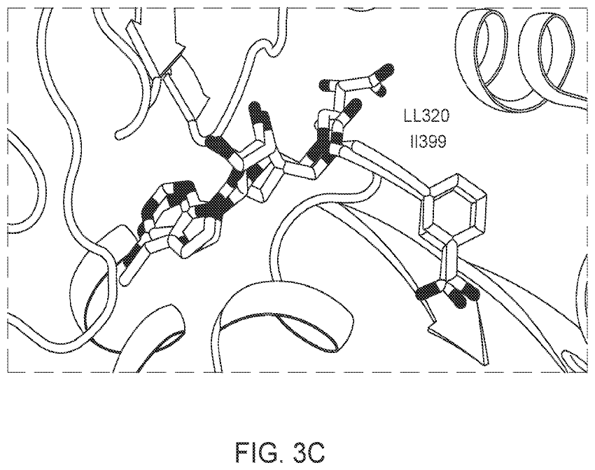

To elucidate the molecular interactions between II399 and NNMT, the X-ray co-crystal structure of NNMT-II399 was determined (FIG. 3A). Full-length hNNMT wild type and triple mutant K100A E101A E103A (hNNMTtm) were expressed and purified as previously reported Peng et al., Biochem 50: 7800-7808 (2011)). Briefly, full-length hNNMT (amino acids 1-270)) cloned in pET28a(+) with an N-terminal TEV cleavage site was synthesized by Genscript for biochemical assays. The full-length hNNMTtm cloned in pET28a-LIC obtained from Addgene (#40734) was used for crystallization. Protein was expressed in Escherichia coli BL21-CodonPlus (DE3)-RIPL competent cells and induced by 0.3 mM isopropyl-D-1-thiogalactopyranoside at 16° C. for 20 hours. Harvested cells were resuspended in ten volumes of 50 mM KH2PO4/K2HPO4 (pH=7.4) with 500 mM NaCl, 25 mM imidazole, 5 mM β-mercaptoethanol, and 100 μM PMSF, then lysed through sonication (Qsonica Q55 cell disruptor) on ice at 80% power using 5-7 cycles of 30-second pulse, 30-second rest. The cell lysate was centrifuged at 25,000 g for 30 minutes at 4° C., and the supernatant was then applied to two 1 mL HiTrap FF Ni-NTA columns connected in series on a GE AKTA Prime purification system using 50 mM KH₂PO₄/K₂HPO₄ (pH=7.4) with 500 mM NaCl and 0.5 mM TCEP, washed and eluted using a step-gradient of imidazole (0.025, 0.05, 0.1, 0.25, and 0.5 M). The peak fractions were verified by SDS-PAGE analysis, and the purest fractions were combined. For enzymatic assays, combined His-NNMT was dialyzed in the dialysis buffer (25 mM Tris, pH=7.5, 150 mM NaCl, 50 mM KCl) and concentrated to 1.5 mg/mL for biochemical assays. For crystallography study, the sample was then applied to an S200 Sephacryl HR size exclusion column (26/100, GE) using 50 mM Tris-HCl, pH=8.0, 100 mM NaCl, 0.5 mM TCEP, 5% glycerol. Fractions containing purest NNMT were combined, concentrated to 0.8 mg/mL, and supplemented with additional TCEP to a final concentration of 1 mM. G9a, SETD7, PRMT1, TbPRMT7, NTMT1, PNMT, and INMT were expressed and purified as previously reported (Babault et al., J Med Chem 61: 1541-1551 (2018); Policarpo et al., J Med Chem 62: 9837-9873 (2019); and Wu et al., PLoS One 5: e8570 (2010)).

For co-crystallization, 12 mg/mL hNNMT triple mutant was mixed with II399 at a 1:4 molar ratio in 50 mM Tris-HCl, pH=8.0, with 0.5 mM TCEP and 5% glycerol, incubated for 1 h at 4° C. Broad matrix crystallization screening was performed using a Mosquito-LCP high throughput crystallization robot (TTP LabTech) using a hanging-drop vapor diffusion method, incubated at 20° C. Crystals containing II399 were grown in 2.0 M ammonium sulfate and 0.1 M sodium HEPES (pH 7.0). Crystals were harvested directly from the 96-well crystallization plates and flash-frozen by plunging into liquid nitrogen. Single crystal diffraction data were collected at GM/CA ID-B beamline at Advanced Photon Source, Argonne National Laboratory. The data were indexed, integrated, and scaled using HKL2000 (Otwinowski et al., Methods Enzymol 276: 307-326 (1997)) followed by molecular replacement with Phaser-MR (PHENIX) (Adams et al., Acta Crystallogr Sect D Biol Crystallogr 66: 213-221 (2010)). Iterative model building and refinement were performed using COOT and phenix. refine (PHENIX), respectively (Emsley et al., Acta Crystallogr Sect D Biol Crystallogr 66: 486-501 (2010)). Figures were prepared using PyMOL (Schrödinger) and the model coordinate and structure factor have been deposited in the Protein Data Bank with ID 7RKL.

Similar to LL320, II399 occupies both the SAM and substrate (NAM) binding pockets according to the superimposed structures. The adenine part of II399 binds similarly to that of SAH. The chloride in adenosine is located about 3 Å from Asp 142, which is within the halogen bonding distance. The N1 in the adenosine moiety, diols on the ring, and benzamide retain the interaction with the backbone of Asp142 and Val143, Asp85, Asn95, Ser201, and Ser213. While the aspartate moiety of LL320 participates in hydrogen bonding with Thr163, Tyr25, Tyr69, and Gly63 of NNMT, these interactions were lost with II399. However, the carbonyl of the acetyl group hydrogen bonds with Tyr20. The benzamide portion of II399 was slightly shifted within the substrate-binding pocket, enabling one hydrogen bond with Ser213 and a new water-mediated hydrogen bond with Asp 197. Notably, Tyr 20 and Asp 197 are important for substrate recognition of NNMT (Babault et al., J Med Chem 61: 1541-1551 (2018)), which may explain the enhanced selectivity of II399 compared to LL320. In addition, Tyr 204 stacks against the phenyl group of benzamide with a distance of about 3.6 Å, allowing for possible pi-pi interactions.

The orientations of the hydroxyl groups enable it to make multiple hydrogen bonding interactions with Asn 90, Asp 85, and the backbone of Asp 85 and Tyr 86. Interestingly, the carbonyl group in the amide has hydrogen bonding interactions with Tyr 20, which is one of the tyrosines in the binding site of NNMT with which the aspartic acid moiety in LL320 interacts. The primary amide in the benzamide portion occupies the substrate pocket and makes crucial hydrogen bonding and hydrophobic interactions with NNMT. The amine and acetyl groups make hydrogen bonding interactions with Ser 201 and Ser 213, respectively. Tyr 204 nicely overlays the aromatic group in benzamide with a distance of about 3.6 Å, making hydrophobic interactions. In addition, the carbonyl group makes water-mediated hydrogen bonding with Asp 197. Compound II399 makes important bonding interactions with Tyr 20 and Asp 197, which are two residues important for substrate recognition of NNMT. Superimposition of II399 with LL320 shows a similar binding pattern between both inhibitors. However, the amide bond in II399 orients the acetyl group differently from the aspartic acid towards a sterically hindered region of the enzymes. This possibly is the reason larger lipophilic groups were not tolerated due to potential steric hindrance. The cyclopentane orients the hydroxyl groups towards the enzyme in contrast with the hydroxyl groups on LL320, thereby making multiple hydrogen bonding interactions with the residues in the binding site of NNMT. The superimposition also suggests that the adenine portion of II399 extends further to interact with NNMT.

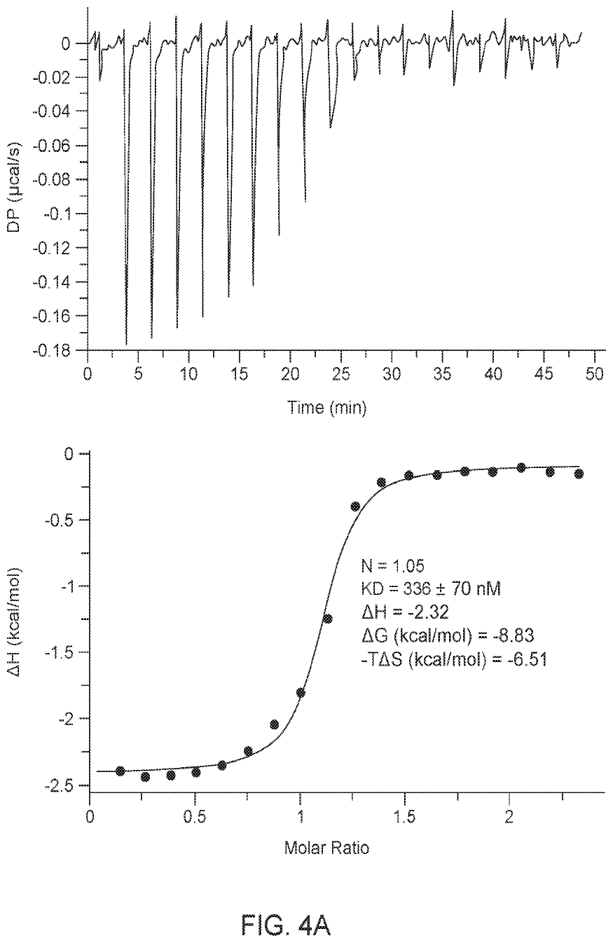

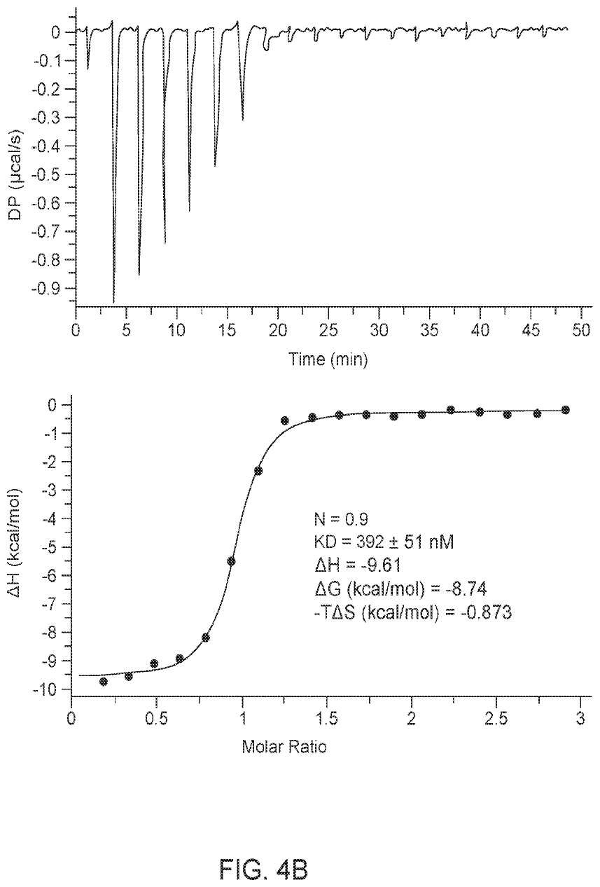

Furthermore, the binding affinity of II399 was measured using isothermal titration calorimetry (ITC) in parallel with LL320 to examine the role of entropy. ITC measurements were performed at 25° C. using a MicroCal PEAQ Instrument (Malvern Instruments). hNNMT was dialyzed in ITC buffer [50 mM Tris, pH 7.5, 100 mM NaCl] supplemented with 1% DMSO for the binding with the compounds overnight and diluted to 50 μM. Compounds II399 and LL320 were prepared in the dialysis buffer at 1 mM. Binding constants were calculated by fitting the data using the ITC data analysis module in Origin 7.0. Overall, II399 had a marginally better K_{d} of 336±70 nM than LL320 with a K_{d} of 392±51 nM (FIGS. 4E and 4F). The change in entropy indicates that rigidifying II399 with the acetyl group reduces entropy loss upon binding; however, there's also a clear compensating decrease in binding enthalpy due to the loss of the many hydrogen bonding interactions that the aspartic acid moiety lends LL320.

TABLE 6
Summary of Crystallographic Data Collection and Refinement.

| Data Collection | NNMT1/II399 | |
|---|---|---|
| λ (Å) | 1.033 | |
| Space group | P1 | |
| a, b, c (Å) | 45.68, 62.03, 106.98 | |
| α, β, γ (°) | 82.94, 81.92, 68.52 | |
| Resolution (Å)* | 41-2.08 | (x-y) |
| Completeness (%)* | 91.0 | (84.6) |
| Redundancy* | 3.6 | (3.4) |
| R_{sym} ^{†}* | 0.151 | (0.756) |
| I/σ (I)* | 8.5 | (1.5) |
| CC_{1/2} | 0.98 | (0.58) |
| Refinement | | |
| Resolution (Å) | 2.08 | |
| No. reflections | 59041 | |
| R^{§}/R_{free}^{¶} | 0.20/0.24 | |
| r.m.s. deviations | | |
| Bonds (Å) | 0.004 | |
| Angles (°) | 0.707 | |
| No. Protein atoms | 7918 | |
| No. Ligand atoms | 136 | |
| No. Waters | 684 | |
| B-factors (Å²) | | |
| Wilson B | | |
| Protein | 27.12 | |
| Ligands | 20.53 | |
| Waters | 35.86 | |
| Ramachandran | | |
| Analysis^{¥} | | |

| | |
|---|---|
| Favored (%) | 97.78 |
| Allowed (%) | 2.22 |
| Outliers (%) | 0 |
| PDB code | 7RKL |

^{†} R_{sym} = Σ_{hkl, j} (|Iₕₖₗ − <I>|)/Σ_{hkl, j} Iₕₖₗ, where <Iₕₖₗ> is the average intensity for a set of j symmetry related reflections and Iₕₖₗ is the value of the intensity for a single reflection within aset of symmetry-related reflections.
^{§}R factor = Σₕₖₗ (∥Fₒ| − |F_{c}∥)/Σₕₖₗ|Fₒ| where Fₒ is the observed structure factor amplitude and F_{c} is the calculated structure factor amplitude.
^{¶}R_{free} = Σ_{hkl, T} (∥Fₒ| − |F_{c}∥)/Σ_{hkl, T}|Fₒ|, where a test set, T (5% of the data), is omitted from the refinement.
^{¥}Performed using Molprobity within PHENIX.
*Indicates statistics for last resolution shell shown in parenthesis.

### Example 10 Molecular Docking of II559 and II562

To understand how the substituents in the benzamide group drives the improvement in inhibitory affinity, molecular docking was performed on two top potent inhibitors II559 and II562. The model predicted two additional interactions in II559 contributed by the chloride ortho to the benzamide: halogen bonding between the chloride and Tyr24 in NNMT and enhanced van der Waals interactions between Tyr204 and the benzamide. The methyl group in II562 was also predicted to make hydrophobic interactions with Ala198 and Ala247 within the binding pocket of NNMT (FIGS. 8E-8G).

### Example 11 Inhibition on Cellular MNA Levels

To test the cell uptake of II399, we checked the cellular concentration of II399 through MS detection after incubating it with cells for 5 hours as described before. Ivamu et al Biomolecules 2021, 11, 854. Intriguingly, even 1 μM of II399 was detected inside cells. Compared to LL320 which was not detected at 100 μM,^{[13]} II399 demonstrated over 100-fold improved cell uptake. To examine the cellular potency of II399, we determined its ability to decrease the cellular level of MNA through an LC-MS assay. Human renal cancer cell line 769P was used because of its overexpressed level of NNMT (Ulanovskaya et al., Nat. Chem. Biol. 9: 300-306 (2013)).

The ability of these bisubstrate inhibitors to decrease the MNAM in cells was evaluated in 769P cells. NNMT has been reported to be overexpressed in the 769P cell line. 769-P cells were seeded into a 24-well plate with a density of 0.1×10⁶ cells/well in 500 μL media and incubated at 5% CO₂ and 37° C. overnight for cells to attach. The next day, the inhibitor diluted in media was added at different concentrations including DMSO as a control. The cells were incubated for the required time (24 and 48 hours). At the end of the incubation, media was aspirated, and the cells were washed with cold PBS twice. Then 80 μL of extraction buffer (100% MeOH) containing internal standards (40 ng/ml NAM_{d4} and 20 ng/ml MNA_{d3}) were added to the cells, and the cells were incubated at room temperature for 20 minutes with mild shaking. Finally, 20 μL of ddH₂O were added, and the mixture was mixed and then centrifuged at 5000 g×10 minutes. The supernatant was collected and analyzed by QQQ-MS with a HILIC column together with a precolumn. Mobile phase A was composed of 100% CH₃CN, and mobile phase B was composed of 95:5 (v/v) H₂O:CH₃CN. Both solvents were supplemented with 0.1% formic acid to assist ion formation in a positive mode. The flow rate started at 0.1 ml/min for 5 minutes. The gradient started with 0% B for 5 minutes and increased linearly to 100% B over 40 minutes with a flow rate of 0.4 ml/min, followed by an isocratic gradient of 100% B for 10 minutes at 0.5 ml/min. Then, the column was equilibrated with 0% B for 5 minutes at 0.4 ml/min.

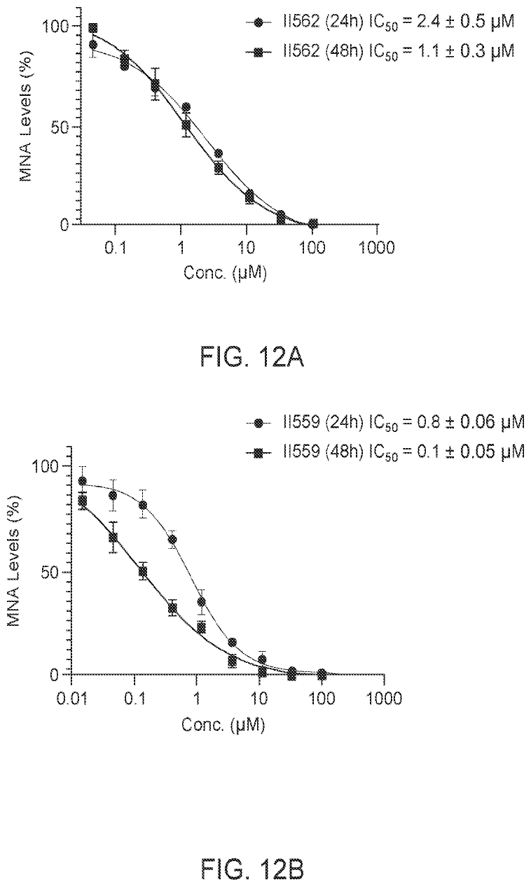

The treatment of 769P cells with II399 led to a cell-based IC₅₀ of 8.8 μM and 2.0 μM at 24 hours and 48 hours, respectively. Though this represents about a 180-fold difference between the biochemical and cellular inhibition in 24 hours and about 40-fold in 48 hours, it also represents progress in the cellular potency of bisubstrate inhibitors. The more lipophilic analogue II562 with a methyl group ortho to the benzamide displayed a cell-based IC₅₀ of 2.4 μM and 1.1 μM at 24 hours and 48 hours, respectively. while the most potent analogue II559 displayed a submicromolar cell-based IC₅₀ of 0.8 μM and 0.1 μM after 24 hours and 48 hours incubation, respectively, with 769P cells (Table 6). This represents a 11-fold and 20-fold difference between the biochemical and cellular inhibition in 24 hours and 48 hours, respectively. The difference between the biochemical and cellular inhibition could be due to limited permeability as indicated by the PAMPA assay. The time-dependent increase in inhibitory activity shows the bisubstrate inhibitors are metabolically stable even up to 48 hours (FIGS. 12A-12B). In addition to being the first reported cell-potent bisubstrate inhibitor of NNMT. II559 is also possesses better cellular potency than currently available substrate competitive inhibitors. Compared to lead compound II399, the chloro analogue II559 exhibited a 10- and 20-fold enhanced cellular inhibition at 24 hours and 48 hours, respectively. Notably. II559 represents the most cell-potent inhibitor for NNMT to date. The ability of these compounds to penetrate the cell, thermally stabilize, and inhibit the methylation function of NNMT has been established.

Indeed, II399 has been used to demonstrate its effect in suppressing palmitate-induced lipotoxicity (Griffiths et al., Am. J. Physiol. Physiol. 321: C585-C595 (2021)). Importantly, the sustained and enhanced cellular inhibition suggests that the inhibitor is metabolically stable and implies the benefit of tight binding. Compared to LL320 and its ester prodrugs which were barely detectable in cells at 100 μM, II399 established the promise of building selective and cell-potent inhibitors for NNMT through the utilization of an assembly of the acetamide, 4-chloro pyrrolopyrimidine, and cyclopentane functional groups. Notably, this is the first example of using an acetyl group to replace the hydrophilic aspartic acid moiety of the SAM cofactor. Furthermore, the strategy described here may have the potential to be applied in the development of new bisubstrate inhibitors or SAM-competitive inhibitors for other SAM-dependent methyltransferases to enhance the cellular potency without compromising binding affinity.

TABLE 6
Cellular inhibition of bisubstrate inhibitors

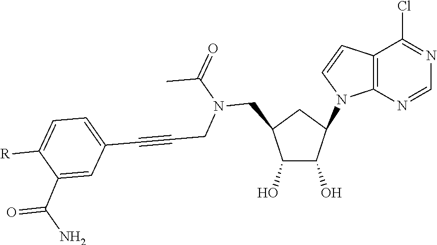

| | | | Cellular IC₅₀ (μM)^{a} | |
|---|---|---|---|---|
| Compound | R | IC₅₀ (μM) | 24 h | 48 h |
| II339 | H | 0.049 ± 0.008 | 8.8 | 2.0 |
| II562 | CH₃ | 0.030 ± 0.001 | 2.4 | 1.1 |
| II559 | Cl | 0.006 ± 0.0001 | 0.8 | 0.1 |

^{a}All IC₅₀ values were performed in duplicates (n = 2) and presented as mean ± SD.

### Example 12 Effect of Inhibition on Viability and Mobility of Renal Cancer Cells

To investigate the effect of NNMT inhibitors on cell growth, II399, II559, and II562 were evaluated in both nonaggressive and aggressive renal cancer cell lines 769P and 786O by AlamarBlue assay, respectively (Ulanovskaya et al., Nat Chem Biol 9(5): 300-306 (2013); and Riss et al., Assay Guide Man 1-25 (2013)). Renal cancer cells were seeded as 5,000 cells/well on 96-well plates in the presence of the inhibitors diluted in media (to a final concentration of 1% DMSO) at different concentrations for the specified time. The media and was changed and the inhibitors replaced every 2 days. Cell viability was assessed using 0.2 mg/ml resazurin solutions prepared from resazurin sodium salt (Acros Organics™, AC418900050) dissolved in sterile 1×PBS. Then, the cells were incubated with 10 μl resazurin solution (10% of cell culture volume) for 3 hours at 37° C. The fluorescence was measured using a CLARIOstar microplate reader (Ex=540 nm, Em=620 nm) at 37° C. Cell viability was calculated as 100%×(fluorescence of treated cells−fluorescence of background controls)/(fluorescence of DMSO controls−fluorescence of background controls).

The effect of NNMT inhibitors on the cell proliferation was evident after 10 days. For 769P cells, only II559 was able to effect complete nonviability of the cells at 300 μM and displayed a GI₅₀ value of 95 μM (FIG. 14A). For the aggressive renal cancer cell line 786O, all three inhibitors demonstrated complete inhibition of the cell viability at the concentration of 300 μM. Compared to II339 with a GI₅₀ of 71 μM, II559 and II562 displayed 6- and 2-fold improved potency on renal cancer cell growth, respectively (FIG. 14B). Interestingly, the trend of cell growth inhibition is consistent with the biochemical and cellular inhibition of NNMT activity as II559 displayed the best growth inhibition with a GI₅₀ of 12 μM. High expression of NNMT in cancer cells has been shown to increase cell migration and invasion Wu et al., Oncogene 27: 6679-6689 (2008)). Metastasis of cancer cells drives both morbidity and mortality in patients (Xi et al., Med Sci Monit 24: 1034-1043 (2018); and Reustle et al., Clin Transl Med 12(6): e883 (2022)). Chemical probes that can inhibit both cell growth and migration will be essential in preventing metastasis.

To investigate if our probes can affect cancer cell mobility in addition to inhibiting cell viability and NNMT, II559 and II562 were evaluated in a wound healing assay with the aggressive renal cancer cell line 786O cells which has higher expression of NNMT. A total of 0.1×10⁶ 786O cells were seeded in a 96-well plate and cultured to 90% confluence. An 800 μm pipette tip was used to scratch the plate using BioTek AUTOSCRATCH. The cells were washed twice before treatment with the inhibitor. The mobility of the cells was observed under the microscope and photographed by Lionheart™ FX Automated Microscope hourly until the wound was completely healed. After 800 μm scratch, the cells treated with NNMT inhibitors at 100 μM displayed about three times slower rate of healing after 12 hours incubation. The cells treated with 50 μM concentration of II559 and II562 also healed about two times slower than the control cells which were quickly repopulated.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, where a compound/composition is substituted with “an” alkyl or aryl, the compound/composition is optionally substituted with at least one alkyl and/or at least one aryl. The term “or” is used to refer to a nonexclusive “or” unless otherwise indicated.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, or within 99% or more of a stated value or of a stated limit of a range.

The terms and expressions, which have been employed, are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

### REFERENCES

Adams et al., Acta Crystallogr Sect D Biol Crystallogr 66: 2313-221 (2010)
Aksoy et al., J. Biol. Chem. 269(20): 14835-14840 (1994)
Allouche, J Comput Chem 32: 174-182 (2012)
Alston et al., Arch. Biochem. Biophys. 260: 601-608 (1988)
Anacker et al., J Comput Chem 35: 634-643 (2014)
Aubert et al., J. Fluor. Chem. 44(3): 361-376 (1989)
Auffinger et al., Proc. Natl. Acad. Sci. USA 101: 16789-16794 (2004)
Avdeef et al., Eur. J. Pharm. Sci. 14(4): 271-280 (2001)
Babault et al., J Med Chem 61: 1541-1551 (2018)
Berthelot et al., Int'l Pat App Pub No. WO/2017/153186 (2017)
Best et al., Biophys J 103: 1045-1051 (2012)
Best et al., J Chem Theory Comput 8: 3257-3273 (2012)
Blake et al., Antimicrob. Agents Chemother. 61(8) (2017)
Brooks et al., J Comput Chem 4: 187-217 (1983)
Brooks et al., J Comput Chem 30: 1545-1614 (2009)
Chen et al., Pharm. Res. 25(7): 1511-1520 (2008)
Chen et al., Acta Crystallogr Sect D Biol Crystallogr 66: 12-21 (2010)
Chen et al., J. Med. Chem. 62: 3773-3779 (2019)
Chen et al., J. Med. Chem. 62: 10783-10797 (2019)
Chlopicki et al., Br. J. Pharmacol. 152: 230-239 (2007)
Chodera et al., Curr. Opin. Struct. Biol. 21: 150-160 (2011)
Coxon et al., J. Med. Chem. 60(5): 1746-1767 (2017)
Davis et al., Nucl Acids Res 35: 375-383 (2007)
Ding et al., J. Chem. Theory Comput. 13: 2501-2510 (2017)
Eastman et al., PLOS Comput Biol 13: 1-17 (2017)
Eckert et al., Nature 569: 723-728 (2019)
Emsley et al., Acta Crystallogr Sect D Biol Crystallogr 66: 486-501 (2010)
Gao et al., J. Med. Chem. 62(14): 6597-6614 (2019)
Gokul et al., Subcell. Biochem. 61: 597-625 (2013)
Griffiths et al., Am. J. Physiol. Physiol. 321: C585-C595 (2021)
Groth et al., European J. Org. Chem. 28: 6314-6320 (2014)
Hong et al., Biochemistry 57: 5775-5779 (2018)
Huang et al., Nat. Struct. Mol. Biol. 17: 646-649 (2010)
Iyamu et al., Anal. Biochem. 604: 113833 (2020)
Iyamu et al., Biomolecules 11: 854 (2021)
Iyamu et al., RSC Med. Chem. 12: 1254-1261 (2021)
Iyamu et al., Angew. Chemie Int. Ed. 202114813: 1-8 (2022)
Jafari et al., Nat. Protoc. 9(9): 2100-2122 (2014)
Jin et al., Adv. Exp. Med. Biol. 754: 3-29 (2013)
Jorgensen et al., J Chem Phys 79: 926-935 (1983)
Jung et al., J. Clin. Invest. 2: 1-23 (2017)
Justyna, Physiol Behav 176: 139-148 (2017)
Kannt et al., Sci. Rep. 8(1): 3660 (2018)
Kansy et al., J. Med. Chem. 41(7), 1007-1010 (1998)
Kaul et al., Antimicrob. Agents Chemother. 59(8): 4845-4855 (2015)
Kirkwood, J. Chem. Phys. 3: 300-313 (1935)
Kocinaj et al., Mol. Neurobiol. 58: 1769-1781 (2021)
Kraus et al., Nature 508: 258-262 (2014)
Lambertucci et al., Bioorg. & Med. Chem. 17: 2812-2822 (2009)
Mahmoodi et al., J. Am. Chem. Soc. 142: 14222-14233 (2020)
Mondal et al., Angew. Chemie-Int. Ed. 58: 12476-12480 (2019)
Neelakantan et al., J Med Chem 60(1): 5015-5028 (2017)
Olsson et al., J Chem theory Comput 7: 525-537 (2011)
Otwinowski et al., Methods Enzymol 276: 307-326 (1997)
Palanichamy et al., Clin. Cancer Res. 23: 2325-2334 (2017)
Parsons et al., Neurosci. Lett. 342: 13-16 (2003)
Paulini et al., ChemMedChem 1: 340-357 (2006)
Peng et al., Biochemistry 50: 7800-7808 (2011)
Pettersen et al., J Comput Chem 25: 1605-1612 (2004)
Pissios et al., Trends Endocrinol Metab 28(5): 340-353 (2017)
Policarpo et al., J Med Chem 62: 9837-9873 (2019)
Reustle et al., Clin Transl Med 12(6): e883 (2022)
Riss et al., Assay Guid Man 1-25 (2013)
Ryckaert et al., J Comput Phys 23: 327-341 (1977)
Steinbach et al., J Comput Chem 15: 667-683 (1994)
Tang et al., Carcinogenesis 32: 138-145 (2011)
Ulanovskaya et al., Nat. Chem. Biol. 9: 300-306 (2013)
U. S. The PyMOL Molecular Graphics System, Vers. 1.8, Schrodinger LLC, New York, NY
van Haren et al., Biomolecules 11(9): 1357 (2021)
Vanommeslaeghe et al., J Chem Inf Model 52: 3144-3154 (2012)
Vilseck et al., J. Chem. Theory Comput. 17: 3895-3907 (2021)
Wenner, Physiol Behav 176: 100-106 (2018)
Wu et al., Oncogene 27: 6679-6689 (2008)
Wu et al., PLOS One 5: e8570 (2010)
Xi et al., Med Sci Monitor 24: 1034-1043 (2018)
Xuefeng Chen et al., Physiol Behav 176: 139-148 (2011)
Zhang et al., Chem Heterocycl Comp 54: 638-642 (2018)

1. A compound having the formula (I): 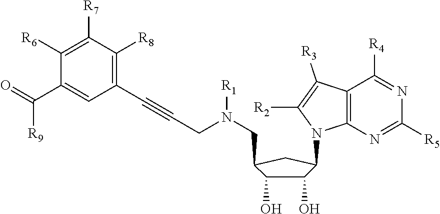
or a pharmaceutically acceptable salt thereof, wherein,
R₁ is an alkyl, a haloalkyl, an alkenyl, an alkynyl, an aminoalkanoic acid, an acyl, an arylalkylacyl, an arylacyl, a cycloalkyl, a heterocyclyl, an aryl, a heteroaryl, an arylalkyl, or a heteroarylalkyl;
R₂, R₃, R₄, and R₅ are, independently, hydrogen, a halo, a haloalkyl, an alkyl, an amino, an alkylamino, or an alkylaminoalkyl; and
R₆, R₇, and R₈ are, independently, hydrogen, methyl, hydroxyl, methoxyl, trifluoromethyl, or a halo; and
R₉ is an amine or methyl.

2. The compound of claim 1, which is: 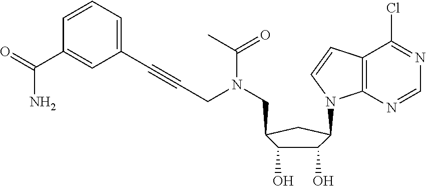 [SMILES: CC(=O)N(CC#Cc1cccc(C(N)=O)c1)C[C@H]1C[C@@H](n2ccc3c(Cl)ncnc32)[C@H](O)[C@@H]1O]
or a pharmaceutically acceptable salt thereof.

3. The compound of claim 1, which is: 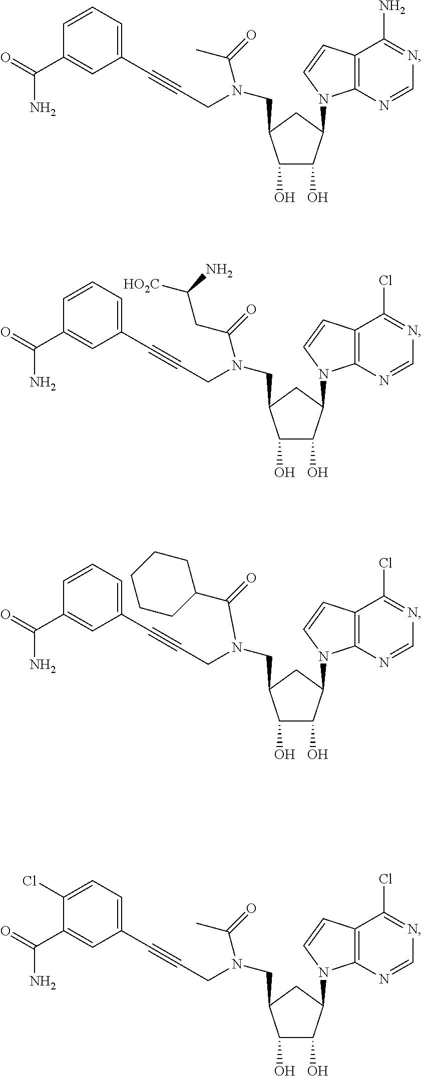 [SMILES: CC(=O)N(CC#Cc1cccc(C(N)=O)c1)C[C@H]1C[C@@H](n2ccc3c(N)ncnc32)[C@H](O)[C@@H]1O ; NC(=O)c1cccc(C#CCN(C[C@H]2C[C@@H](n3ccc4c(Cl)ncnc43)[C@H](O)[C@@H]2O)C(=O)C[C@H](N)C(=O)O)c1 ; NC(=O)c1cccc(C#CCN(C[C@H]2C[C@@H](n3ccc4c(Cl)ncnc43)[C@H](O)[C@@H]2O)C(=O)C2CCCCC2)c1 ; CC(=O)N(CC#Cc1ccc(Cl)c(C(N)=O)c1)C[C@H]1C[C@@H](n2ccc3c(Cl)ncnc32)[C@H](O)[C@@H]1O] 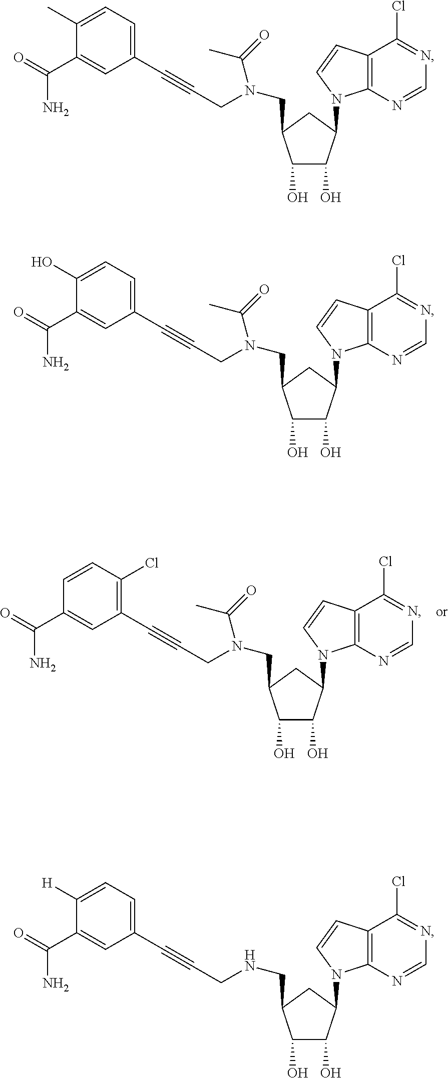 [SMILES: CC(=O)N(CC#Cc1ccc(C)c(C(N)=O)c1)C[C@H]1C[C@@H](n2ccc3c(Cl)ncnc32)[C@H](O)[C@@H]1O ; CC(=O)N(CC#Cc1ccc(O)c(C(N)=O)c1)C[C@H]1C[C@@H](n2ccc3c(Cl)ncnc32)[C@H](O)[C@@H]1O ; CC(=O)N(CC#Cc1cc(C(N)=O)ccc1Cl)C[C@H]1C[C@@H](n2ccc3c(Cl)ncnc32)[C@H](O)[C@@H]1O ; NC(=O)c1cccc(C#CCNC[C@H]2C[C@@H](n3ccc4c(Cl)ncnc43)[C@H](O)[C@@H]2O)c1]
or a pharmaceutically acceptable salt thereof.

4-10. (canceled)

11. The compound of claim 1, wherein said compound is a cell-potent inhibitor for nicotinamide N-methyltransferase (NNMT), a bisubstrate for NNMT and its cofactor, and selective for NNMT over other closely related methyltransferases.

12. A pharmaceutical composition comprising one or more compounds of claim 1, together with one or more pharmaceutically acceptable diluents, excipients, or carriers.

13. The pharmaceutical composition of claim 12, wherein the one or more compounds are cell-potent inhibitors for nicotinamide N-methyltransferase (NNMT), bisubstrates for NNMT and its cofactor, and selective for NNMT over other closely related methyltransferases.

14. A method for inhibiting nicotinamide N-methyltransferase (NNMT) in a subject in need thereof, which method comprises administering to the subject an NNMT-inhibiting effective amount of a compound of claim 1 or a pharmaceutical composition comprising same, together with one or more pharmaceutically acceptable diluents, excipients, or carriers, whereupon NNMT in the subject is inhibited.

15. The method of claim 14, wherein the compound is a cell-potent inhibitor for NNMT, a bisubstrate for NNMT and its cofactor, and selective for NNMT over other closely related methyltransferases.

16. The method of claim 14, wherein the subject has cancer.

17. The method of claim 14, wherein the subject has diabetes, a liver disease, scleroderma, or Parkinson's disease.

18. The method of claim 15, wherein the subject has cancer.

19. The method of claim 15, wherein the subject has diabetes, a liver disease, scleroderma, or Parkinson's disease.

20. A compound of the formula: 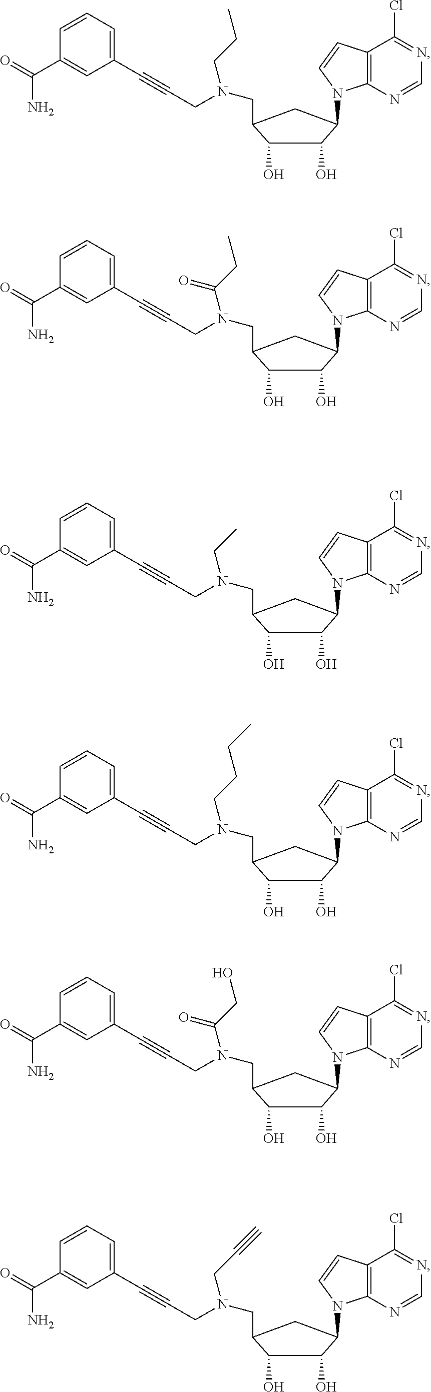 [SMILES: CCCN(CC#Cc1cccc(C(N)=O)c1)CC1C[C@@H](n2ccc3c(Cl)ncnc32)[C@H](O)[C@@H]1O ; CCC(=O)N(CC#Cc1cccc(C(N)=O)c1)CC1C[C@@H](n2ccc3c(Cl)ncnc32)[C@H](O)[C@@H]1O ; CCN(CC#Cc1cccc(C(N)=O)c1)CC1C[C@@H](n2ccc3c(Cl)ncnc32)[C@H](O)[C@@H]1O ; CCCCN(CC#Cc1cccc(C(N)=O)c1)CC1C[C@@H](n2ccc3c(Cl)ncnc32)[C@H](O)[C@@H]1O ; NC(=O)c1cccc(C#CCN(CC2C[C@@H](n3ccc4c(Cl)ncnc43)[C@H](O)[C@@H]2O)C(=O)CO)c1 ; C#CCN(CC#Cc1cccc(C(N)=O)c1)CC1C[C@@H](n2ccc3c(Cl)ncnc32)[C@H](O)[C@@H]1O] 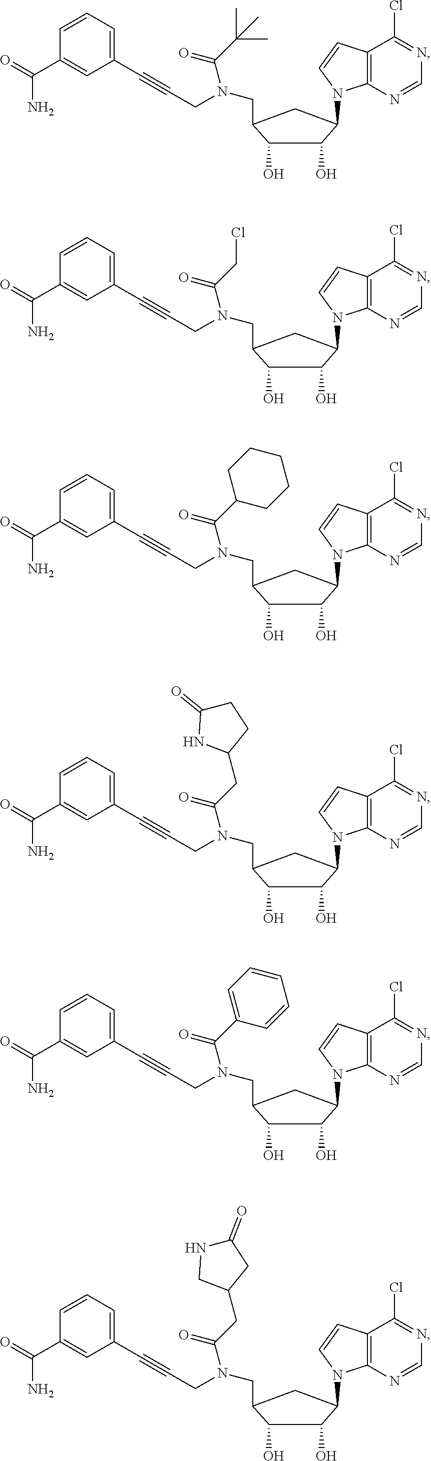 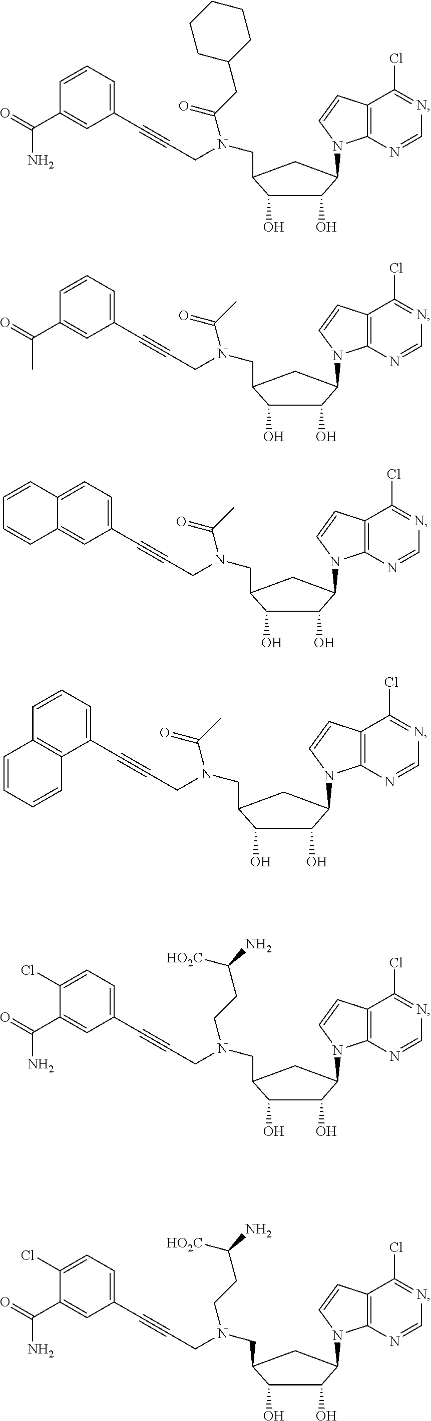 [SMILES: NC(=O)c1cccc(C#CCN(CC2C[C@@H](n3ccc4c(Cl)ncnc43)[C@H](O)[C@@H]2O)C(=O)CC2CCCCC2)c1 ; CC(=O)c1cccc(C#CCN(CC2C[C@@H](n3ccc4c(Cl)ncnc43)[C@H](O)[C@@H]2O)C(C)=O)c1 ; CC(=O)N(CC#Cc1ccc2ccccc2c1)CC1C[C@@H](n2ccc3c(Cl)ncnc32)[C@H](O)[C@@H]1O ; CC(=O)N(CC#Cc1cccc2ccccc12)CC1C[C@@H](n2ccc3c(Cl)ncnc32)[C@H](O)[C@@H]1O ; NC(=O)c1cc(C#CCN(CC[C@H](N)C(=O)O)CC2C[C@@H](n3ccc4c(Cl)ncnc43)[C@H](O)[C@@H]2O)ccc1Cl ; NC(=O)c1cc(C#CCN(CC[C@H](N)C(=O)O)C[C@H]2C[C@@H](n3ccc4c(Cl)ncnc43)[C@H](O)[C@@H]2O)ccc1Cl] 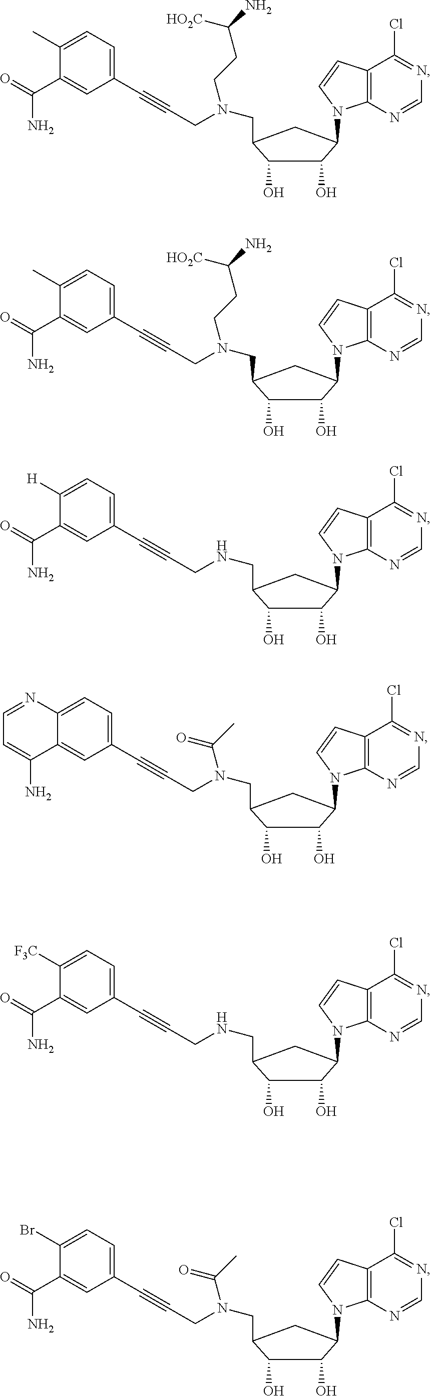 [SMILES: Cc1ccc(C#CCN(CC[C@H](N)C(=O)O)CC2C[C@@H](n3ccc4c(Cl)ncnc43)[C@H](O)[C@@H]2O)cc1C(N)=O ; Cc1ccc(C#CCN(CC[C@H](N)C(=O)O)C[C@H]2C[C@@H](n3ccc4c(Cl)ncnc43)[C@H](O)[C@@H]2O)cc1C(N)=O ; NC(=O)c1cccc(C#CCNCC2C[C@@H](n3ccc4c(Cl)ncnc43)[C@H](O)[C@@H]2O)c1 ; CC(=O)N(CC#Cc1ccc2nccc(N)c2c1)CC1C[C@@H](n2ccc3c(Cl)ncnc32)[C@H](O)[C@@H]1O ; NC(=O)c1cc(C#CCNCC2C[C@@H](n3ccc4c(Cl)ncnc43)[C@H](O)[C@@H]2O)ccc1C(F)(F)F ; CC(=O)N(CC#Cc1ccc(Br)c(C(N)=O)c1)CC1C[C@@H](n2ccc3c(Cl)ncnc32)[C@H](O)[C@@H]1O] 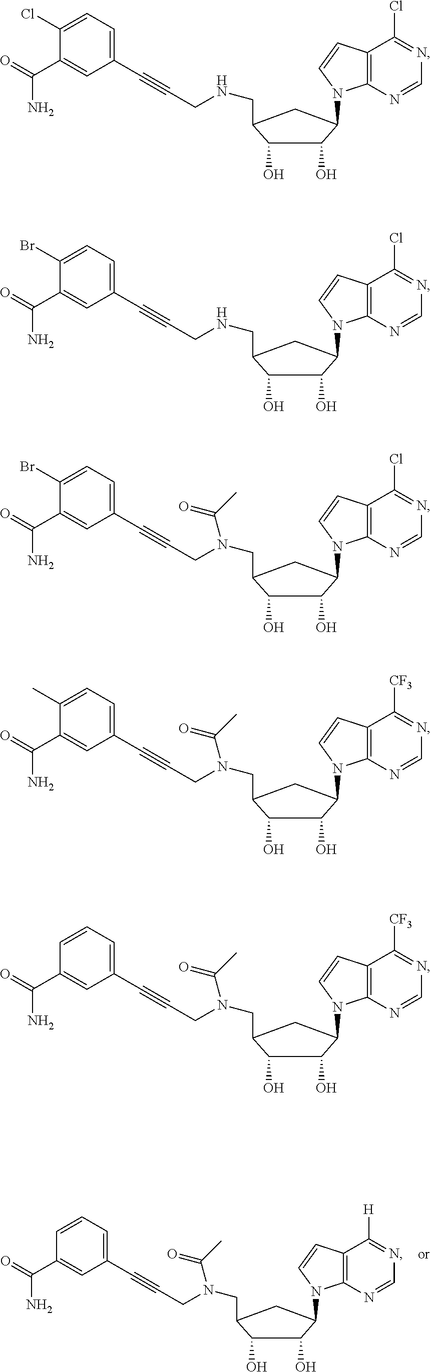 [SMILES: NC(=O)c1cc(C#CCNCC2C[C@@H](n3ccc4c(Cl)ncnc43)[C@H](O)[C@@H]2O)ccc1Cl ; NC(=O)c1cc(C#CCNCC2C[C@@H](n3ccc4c(Cl)ncnc43)[C@H](O)[C@@H]2O)ccc1Br ; CC(=O)N(CC#Cc1ccc(Br)c(C(N)=O)c1)CC1C[C@@H](n2ccc3c(Cl)ncnc32)[C@H](O)[C@@H]1O ; CC(=O)N(CC#Cc1ccc(C)c(C(N)=O)c1)CC1C[C@@H](n2ccc3c(C(F)(F)F)ncnc32)[C@H](O)[C@@H]1O ; CC(=O)N(CC#Cc1cccc(C(N)=O)c1)CC1C[C@@H](n2ccc3c(C(F)(F)F)ncnc32)[C@H](O)[C@@H]1O ; CC(=O)N(CC#Cc1cccc(C(N)=O)c1)CC1C[C@@H](n2ccc3cncnc32)[C@H](O)[C@@H]1O] 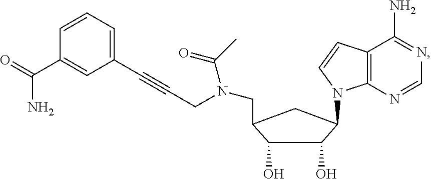 [SMILES: CC(=O)N(CC#Cc1cccc(C(N)=O)c1)CC1C[C@@H](n2ccc3c(N)ncnc32)[C@H](O)[C@@H]1O]
or a pharmaceutically acceptable salt thereof. or a pharmaceutical composition comprising same, together with one or more pharmaceutically acceptable diluents, excipients, or carriers.

21. The compound of claim 20, wherein the compound is a cell-potent inhibitor for nicotinamide N-methyltransferase (NNMT), a bisubstrate for NNMT and its cofactor, and selective for NNMT over other closely related methyltransferases.

22. A method for inhibiting nicotinamide N-methyltransferase (NNMT) in a subject in need thereof, which method comprises administering to the subject an NNMT-inhibiting effective amount of a compound of claim 21 or a pharmaceutical composition comprising same, whereupon NNMT in the subject is inhibited.

23. The method of claim 22, wherein the subject has cancer.

24. The method of claim 22, wherein the subject has diabetes, a liver disease, scleroderma, or Parkinson's disease.
